(19) United States
(12) Patent Application Publication
(10) Pub. No.: US 2011/0212104 A1
(43) Pub. Date: Sep. 1, 2011

(54) INFLAMMATORY BOWEL DISEASE BIOMARKERS AND RELATED METHODS OF TREATMENT

(75) Inventors: Maribel Beaumont, San Francisco, CA (US); Corinne Cayatte, San Francisco, CA (US)

(73) Assignee: Schering Corporation, Kenilworth, NJ (US)

(21) Appl. No.: 13/127,212

(22) PCT Filed: Oct. 29, 2009
(86) PCT No.: PCT/US09/62541

§ 371 Date: May 2, 2011

Related U.S. Application Data
(60) Provisional application No. 61/110,756, filed on Nov. 3, 2008.
(60) Provisional application No. 61/225,847, filed on Jul. 15, 2009.

(51) Int. Cl. A61K 39/395; G01N 33/566; C07K 16/18; C07K 16/24; A61P 29/00; A61P 1/00

(52) U.S. Cl. 424/158.1; 436/501; 435/7.92; 424/173.1; 530/387.3

(57) ABSTRACT

Biomarkers associated with inflammatory bowel disease (IBD) are provided, as well as methods of using such biomarkers for diagnosing, assessing and monitoring disease progression. The biomarkers may be measured at the protein level or the gene expression level Biomarkers may be tracked individually or in groups of two or more. The disclosed biomarkers may find particular utility in monitoring a course of therapy, such as treatment with an IL-23 antagonist. Changes in biomarker levels can also be used to confirm target engagement and therapeutic efficacy. Changes in biomarkers can also be used inform modification of a therapeutic regimen, for example to increase or decrease dosing of a therapeutic agent, such as an anti-IL-23 or anti-IL-23R anti-body.

The Sequence Listing filed electronically herewith is also hereby incorporated by reference in its entirety (File Name: BP06867L02US_SeqListing.txt; Date Created: Oct. 27, 2009; File Size: 116 KB.)

### FIELD OF THE INVENTION

The present invention relates generally to diagnosing and monitoring the progression of inflammatory bowel disease in a subject, for example monitoring the course of treatment with a drug. Such drug may inhibit the activity of IL-23, and may be a monoclonal antibody. Diagnosis and monitoring are achieved by measuring the level of biomarkers at the protein and/or gene expression level.

### BACKGROUND OF THE INVENTION

The immune system functions to protect individuals from infective agents, e.g., bacteria, multi-cellular organisms, and viruses, as well as from cancers. This system includes several types of lymphoid and myeloid cells such as monocytes, macrophages, dendritic cells (DCs), eosinophils, T cells, B cells, and neutrophils. These lymphoid and myeloid cells often produce signaling proteins known as cytokines. The immune response includes inflammation, i.e., the accumulation of immune cells systemically or in a particular location of the body. In response to an infective agent or foreign substance, immune cells secrete cytokines which, in turn, modulate immune cell proliferation, development, differentiation, or migration. Immune response can produce pathological consequences, e.g., when it involves excessive inflammation, as in the autoimmune disorders. See, e.g., Abbas et al. (eds.) (2000) Cellular and Molecular Immunology, W.B. Saunders Co., Philadelphia, Pa.; Oppenheim and Feldmann (eds.) (2001) Cytokine Reference, Academic Press, San Diego, Calif.; von Andrian and Mackay (2000) New Engl. J. Med. 343:1020-1034; Davidson and Diamond (2001) New Engl. J. Med 345:340-350.

Crohn's disease (CD) and ulcerative colitis (UC), collectively Inflammatory Bowel Disease (IBD) are chronic, inflammatory diseases of the gastrointestinal tract. Both disorders are characterized by abdominal pain, diarrhea (often bloody), a variable group of “extra-intestinal” manifestations (such as arthritis, uveitis, skin changes, etc.) and the accumulation of inflammatory cells within the small intestine and colon. Additional symptoms, aspects, manifestations, or signs of IBD include malabsorption of food, altered bowel motility, infection, fever, rectal bleeding, weight loss, signs of malnutrition, perianal disease, abdominal mass, and growth failure, as well as intestinal complications such as stricture, fistulas, toxic megacolon, perforation, and cancer, and including endoscopic findings, such as friability, aphthous and linear ulcers, cobblestone appearance, pseudopolyps, and rectal involvement and, in addition, anti-yeast antibodies. See, e.g., Podolsky (2002) New Engl. J. Med. 347:417-429; Hanauer (1996) New Engl. J. Med. 334:841-848; Horwitz and Fisher (2001) New Engl. J. Med. 344:1846-1850.

IBD affects both children and adults, and has a bimodal age distribution (one peak around 20, and a second around 40). IBD is a chronic, lifelong disease, and is often grouped with “autoimmune” disorders (e.g. rheumatoid arthritis, type I diabetes mellitus, multiple sclerosis, etc.). IBD is found almost exclusively in the industrialized world. An estimated one million Americans are believed to have IBD, half of which have CD and half of which have U C. Hanauer (2006) Inflamm. Bowel Dis. 12:S3. There is an unexplained trend towards increasing incidence of IBD, particularly Crohn's Disease, in the U.S. and Europe.

Treatment of IBD is varied. First line therapy typically includes salicylate derivatives (e.g. 5-ASA) given orally or rectally. Corticosteroids are also used, despite the untoward side-effects, as well as immunomodulators such as azathioprine and 6-mercaptopurine. Newer treatment options include anti-metabolites (e.g. methotrexate, 6-mercaptopurine) and immunomodulators, such as infliximab (Remicade®), a chimeric antibody directed to TNF-α, and other TNF-α antagonists such as adalimumab (Humira®), certolizumab pegol (Cimzia®), golimumab (Simponi), and natalizumab (Tysabre®).

Inflammatory bowel disorders are mediated by cells of the immune system and by cytokines. For example, Crohn's disease is associated with increased IL-12 and IFNγ, while ulcerative colitis is associated with increased IL-5, IL-13, and transforming growth factor-beta (TGF-β). IL-17 expression may also increase in Crohn's disease and ulcerative colitis. See, e.g., Podolsky (2002) New Engl. J. Med. 347:417-429; Bouma and Strober (2003) Nat. Rev. Immunol. 3:521-533; Bhan et al. (1999) Immunol. Rev. 169:195-207; Hanauer (1996) New Engl. J. Med. 334:841-848; Green (2003) The Lancet 362:383-391; McManus (2003) New Engl. J. Med. 348:2573-2574; Horwitz and Fisher (2001) New Engl. J. Med 344:1846-1850; Andoh et al. (2002) Int. J. Mol. Med. 10:631-634; Nielsen et al. (2003) Scand. J. Gastroenterol. 38:180-185; Fujino et al. (2003) Gut 52:65-70.

Interleukin-12 (IL-12) is a heterodimeric molecule composed of p35 and p40 subunits. Studies have indicated that IL-12 plays a critical role in the differentiation of naïve T cells into T-helper type 1 CD4⁺ lymphocytes that secrete IFNγ. It has also been shown that IL-12 is essential for many T cell dependent immune and inflammatory responses in vivo. See, e.g., Cua et al. (2003) Nature 421:744-748. The IL-12 receptor is composed of IL-12Rβ1 and IL-12Rβ2 subunits. See Presky et al. (1996) Proc. Nat'l Acad. Sci. USA 93:14002.

Interleukin-23 (IL-23) is a heterodimeric cytokine comprised of two subunits, p19 which is unique to IL-23, and p40, which is shared with IL-12. The p19 subunit is structurally related to IL-6, granulocyte-colony stimulating factor (G-CSF), and the p35 subunit of IL-12. IL-23 mediates signaling by binding to a heterodimeric receptor, comprised of IL-23R which is unique to IL-23 receptor, and IL-12Rβ1, which is shared with the IL-12 receptor. See Parham et al. (2000) J. Immunol. 168:5699.

A number of early studies demonstrated that the consequences of a genetic deficiency in p40 in a p40 knockout (KO) mouse were more severe than those found in a p35 KO mouse. Some of these results were eventually explained by the discovery of IL-23, and the realization that the p40 KO prevents expression of not only IL-12, but also of IL-23. See, e.g., Oppmann et al. (2000) Immunity 13:715-725; Wiekowski et al. (2001) J. Immunol. 166:7563-7570; Parham et al. (2002) J. Immunol. 168:5699-708; Frucht (2002) Sci STKE 2002, E1-E3; Elkins et al. (2002) Infection Immunity 70:1936-1948.

Recent studies, through the use of p40 KO mice, have suggested that blockade of both IL-23 and IL-12 is an effective treatment for various inflammatory and autoimmune disorders. However, the blockade of IL-12 through p40 may have adverse systemic consequences, such as increased susceptibility to opportunistic microbial infections. Bowman et al. (2006) Curr. Opin. Infect. Dis. 19:245.

IL-23R has been implicated as a critical genetic factor in the inflammatory bowel disorder. Duerr et al. (2006) Science 314:1461. A genome-wide association study found that the gene for IL-23R was highly associated with Crohn's disease, with an uncommon coding variant (Arg381Gln) conferring strong protection against the disease. This genetic association confirms prior biological findings (Yen et al. (2006) J. Clin. Investigation 116:1218) suggesting that IL-23 and its receptor (including IL-23R) are promising targets for new therapeutic approaches to treating IBD.

Therapeutic antibodies may be used to block cytokine activity. A significant limitation in using antibodies as a therapeutic agent in vivo is the immunogenicity of the antibodies. As most monoclonal antibodies are derived from rodents, repeated use in humans results in the generation of an immune response against the therapeutic antibody. Such an immune response may range from a loss of therapeutic efficacy to a fatal anaphylactic response. Initial efforts to reduce the immunogenicity of rodent antibodies involved the production of chimeric antibodies, in which mouse variable regions were fused with human constant regions. Liu et al. (1987) Proc. Natl. Acad. Sci. USA 84:3439-43. Additional advances in the field have led to humanized antibodies, in which all of the antibody sequences other than the complementarity determining regions (CDRs) are replaced with human sequences to minimize the immunogenicity of the therapeutic antibody. Human antibodies have also been developed in which all sequences are of human origin.

Research into treatment of IBD requires that patients be monitored to assess the effectiveness of various therapeutic regimens and agents. Such monitoring is also useful in directing the course of therapy for patients in the clinic. Self-reporting of symptoms is inherently subjective and non-quantitative. Langhorst et al. (2008) Am. J. Gastroenterol. 103:162; Cooney et al. (2007) Trials 8:17. More invasive techniques, such as sigmoidoscopy, colonoscopy and biopsy, are uncomfortable, inconvenient and expensive, as are imaging techniques such as Barium X-ray or CT scan. See, e.g, Winther et al. (1998) Drugs of Today 34:935. Without a reliable measure of disease progression it is difficult to evaluate the effectiveness of a therapeutic regimen, or to optimize dosing for any individual patient. See generally Carty & Rampton (2003) Brit. J. Clin. Pharmacol. 56:351.

Assays for calprotectin in feces have been approved for diagnosis and monitoring of IBD. Von Roon et al. (2007) Am. J. Gastroenterol. 102:803. Blood tests to assess general systemic inflammatory markers can also be used, such as inflammatory cell count, C-reactive protein (CRP), orosomucoid, haptoglobin and erythrocyte sedimentation rate, as well as measurements of nutrient deficiency, electrolytes and albumin. Serum markers that have previously been associated with IBD relapse include α-1 glycoprotein, α-1 antitrypsin, γ-globulin, orosomucoid, and fibrinogen, but such markers tend to have low sensitivity and specificity. Amott et al. (2002) Aliment. Pharmacol. Ther. 16:857. Successful treatment of Crohn's patients with anti-TNF-α antibodies has been associated with changes in the levels of α-1-acid glycoprotein (orosomucoid), haptoglobin, cholinesterase and prealbumin. Kupcová et al. (2003) Physiol. Res. 52:89.

Panels of genes for use as biomarkers in ulcerative colitis, specifically for use in the identification of non-responders to anti-TNF-α agents and in monitoring treatment, are disclosed at WO 2008/028044 and WO 2008/028031. A panel of genes for use as biomarkers in psoriasis, specifically for use in the identification of non-responders to an anti-IL-12/23 p40 antibody and in monitoring treatment, is disclosed at WO 2007/076521

The need exists for improved methods for monitoring the progression of IBD. Such methods would preferably allow objective determination of a subject's disease state by detection of the level of one or more genes or gene products (biomarkers) reflecting disease severity. Preferably, said biomarkers would also reflect disease status in subjects treated with one or more therapeutic agents for treatment of IBD, such as IL-23 antagonists.

### SUMMARY OF THE INVENTION

The present invention meets these needs in the art and more by providing sets of genes and gene products (“biomarkers”) whose level reflects disease state for IBD. The biomarker levels also reflect disease state in subjects treated with IL-23 antagonists, such as anti-IL-23p19 or anti-IL-23R antibodies.

In one aspect, the invention provides sets of one, two or more biomarkers that can be used to assess the progression or disease state of IBD, and methods for use of the sets of biomarkers. In various embodiments, the invention provides for IBD assessment using one, two, three, four, five, six, seven or more of the biomarkers disclosed herein. In one embodiment, the method does not require handling of fecal samples.

In one embodiment, the one, two or more biomarkers are selected from the group consisting of CCL20, DMBT1, MIF, LCN2 (lipocalin 2), PAP, S100A8/A9 (calprotectin), interleukin-22 (IL-22), haptoglobin, interleukin-6 (IL-6), interleukin-17 (IL-17), lactoferrin, GP-39 (YKL-40), GPX-2, GPX-3, neutrophil elastase, TNF-α and C-reactive protein (CRP). In another embodiment, the one, two or more biomarkers are selected from the group consisting of S100A8/A9 (calprotectin), PAP, LCN2 (lipocalin 2), MIF, DMBT1, interleukin-22 (IL-22), haptoglobin and CCL20. In yet another embodiment, the one, two or more biomarkers are selected from the group consisting of S100A8/A9 (calprotectin), PAP, LCN2 (lipocalin 2), MIF and DMBT1.

In various embodiments the one or more biomarkers are detected in blood (including plasma or serum) or in feces (e.g. a stool sample), or alternatively at least one biomarker is detected in the blood, plasma or serum, and at least one other biomarker is determined in feces. In other embodiments, the one or more biomarkers are detected in tissue samples, such as a biopsy.

In one embodiment, the set of biomarkers comprises CRP, IL-6, IL-17, IL-22, LCN2, CCL20, PAP and S100A8/S100A9, and the levels of the biomarkers are determined in serum. In another embodiment, the set of biomarkers comprises IL-22, PAP and S100A8/A9, and the levels of the biomarkers are determined in serum. In yet another embodiment, the set of biomarkers comprises CRP, IL-6, IL-22 and S100A8/S100A9, and the levels of the biomarkers are determined in serum.

In another embodiment, the set of biomarkers comprises IL-17, IL-22, lactoferrin, PAP and S100A8/S100A9, and the levels of the biomarkers are determined in feces. In a further embodiment, the set of biomarkers comprises PAP and S100A8/S100A9, and the levels of the biomarkers are determined in feces. In yet a further embodiment, the set of biomarkers comprises IL-17, lactoferrin and S100A8/S100A9, and the levels of the biomarkers are determined in feces.

In yet a further embodiment, the set of biomarkers comprises S100A8/A9 and IL-22, and the levels of the biomarkers are determined in serum and/or feces.

In another embodiment, the set of biomarkers comprises CCL20, LCN2, PAP, and optionally GP-39, the levels of the biomarkers are determined in serum, and the inflammatory bowel disease is Crohn's disease. In another embodiment the set of biomarkers suggesting Crohn's disease comprises CCL20, LCN2 and GP-39. In one embodiment, a subject is diagnosed as having Crohn's disease if the level of LCN-2 is greater than about 57 ng/ml in serum, or if the levels of CCL-20 and GP-39 are above about 21 pg/ml and 93 ng/ml in serum, respectively.

In yet another embodiment, the set of biomarkers comprises MIF and GPX-3, the levels of the biomarkers are determined in serum, and the inflammatory bowel disease is ulcerative colitis. In one embodiment the set of biomarkers suggesting ulcerative comprises MIF and LCN2. In one embodiment, a subject is diagnosed as having ulcerative colitis if the level of MIF is greater than about 4.1 ng/ml in serum and the level of LCN2 is greater than about 6.3 ng/ml in serum.

In a diagnostic embodiment, two or more biomarkers selected from the group consisting of CCL20, PAP, GP-39, MIF, and GPX-3 are measured to determine whether a human subject suffers from Crohn's disease as opposed to ulcerative colitis. In such diagnostic embodiments, elevated levels of GPX-3 and MIF, as opposed to CCL20, LCN2, and PAP, suggest ulcerative colitis rather than Crohn's disease. In contrast, elevated levels of CCL20, LCN2, and PAP, as opposed to GPX-3 and MIF, suggest ulcerative colitis rather than Crohn's disease.

In one embodiment biomarker data are evaluated using multivariate discriminate analysis. In another embodiment, biomarker data are evaluated using decision tree analysis.

In some embodiments the method involves detection of biomarker protein levels by immunological detection means, such as ELISA, immunohistochemistry (IHC), fluorescence activated cell sorting (FACS®), Western blot or other immunologically based protein detection method. In other embodiments, the biomarker protein is determined by non-immunological detection means, such as by mass spectroscopic or chromatographic means. In yet other embodiments biomarkers are measured at the gene expression level by gene expression detection means, such as by detecting the level of mRNA, for example using a nucleic acid hybridization-based technique (e.g. an array or chip) or an amplification-based technique (e.g. polymerase chain reaction, TaqMan® real time quantitative PCR analysis).

In other embodiments, the level of a biomarker is judged to be higher or lower than the level in a pre-determined reference sample (e.g. from subject(s) not having IBD, from the subject at some previous time, or from non-diseased tissue from the subject) if it differs by a specified multiple, such as 1.1-, 1.2-, 1.3-, 1.4-, 1.5-, 2-, 3-, 4-, 5-, 7-, 10-, 15-, 20-, 50-, 100-fold or more. In a related embodiment, an absolute level of a biomarker (e.g. a serum concentration) is selected, based on levels detected in known IBD patients and non-IBD controls, as the cut-off for what is considered to be a positive result (i.e. a biomarker level consistent with active IBD).

In another embodiment, samples are judged to differ if the difference in level of expression ratio is similar to the ratios disclosed in Tables 2, 3B or 4B. In general, the “cut-off” for what is considered an “elevated” level of a biomarker will be somewhat less than the ratio of expression in subjects with disease versus non-disease subjects. For example, based on the data in Table 2, the level of MIF might be considered elevated if it is about 3-fold higher than the level in control non-diseased subjects; the level of PAP might be considered “elevated” if it is about 1.5-fold higher than the level in control non-diseased subjects; and the level of calprotectin might be considered “elevated” if it is about 1.3-fold higher than the level in control non-diseased subjects.

In yet other embodiments, the disease state for IBD (CD or UC) is assessed by measurement of two or more biomarkers, such as two, three, four, five or more biomarkers. In some such embodiments, a first determination of disease state is assessed as being higher or lower than a second determination of disease state when the difference in the determinations is greater than the inherent variability of the detection method. In one embodiment, disease state is assessed to be different if the serum levels of one or more of PAP/REG3α, LCN2 and CCL20 are determined to be different. In another embodiment, a subject is diagnosed as having IBD if the serum levels of one or more of PAP/REG3α, LCN2 and CCL20 in the subject are determined to be different from the levels in non-IBD subjects.

Different measurements may be based on samples taken from the same subject at different times, from different subjects (such as IBD and non-IBD subjects, or treated and untreated subjects), from groups of subjects differing in symptom severity, or from different tissues (affected and non-affected) within a single subject.

In another aspect, the invention relates to methods of diagnosing IBD, for example diagnosis of Crohn's disease or diagnosis of ulcerative colitis.

In yet another aspect, the invention relates to methods of assessing target or pathway engagement by a therapeutic agent. Such methods may find use in clinical trials and also in the clinic after regulatory approval. Targets include, but are not limited to, IL-23, IL-23p19, IL-23 receptor, IL-23R and Th17 cells. Pathways include, but are not limited to, IL-23-signaling pathway, the Th17 pathway, or both. In a clinical trial, validation of target engagement is useful in determining whether any failure to achieve therapeutic benefit, if observed, is caused by failure to engage the therapeutic target, as opposed to failure of the intervention in the target pathway to effect a therapeutic benefit. In the clinic, validation of target engagement is useful in determining whether a therapeutic agent is active, for example whether a drug retains its desired biological activity, for example in subjects that do not exhibit apparent benefit or symptom relief.

In various embodiments, one or more of the biomarkers of the present invention is used to track disease progression in subjects undergoing treatment for IBD. The dose, dosing frequency (interval) or other therapeutic parameter may be modified based at least partly on the biomarker determination to ensure that the patient achieves a satisfactory outcome. Such methods are objective, based the results of lab tests rather than the subjective assessment of symptoms by the patient. Patients may be, for example, subjects in a clinical trial or patients receiving an approved treatment.

In some embodiments the treatment is with an IL-23 antagonist. In various embodiments, the IL-23 antagonist is an antibody (or antigen binding fragment thereof) that binds to IL-23 or a subunit thereof, i.e. IL-23p19 or IL-12/23p40, or an antibody that binds to IL-23 receptor or a subunit thereof, i.e. IL-23R or IL-12Rβ1. In various embodiments the antibody is a polyclonal, monoclonal, chimeric, humanized or fully human antibody. In other embodiments the IL-23 antagonist is a nucleic acid molecule, such as an antisense nucleic acid or an siRNA, targeting IL-23 or its receptor, or any subunit of either. In still further embodiments the IL-23 antagonist is a small molecule compound.

In some embodiments, disease progression is tracked in subjects undergoing treatment for IBD, and compared with reference values to assess the efficacy of the treatment regimen, e.g. as a form of screening for therapeutically effective agents. Reference values are obtained, for example, from non-disease subjects or from a matched control group of subjects treated with a placebo. In some embodiments the treatment regimen comprises administration of an IL-23 antagonist.

In various embodiments of the present invention, IBD is evaluated based on general inflammatory markers (e.g. C-reactive protein, CRP) or standard clinical measures (e.g., Crohn's Disease Activity Index, CDAI).

The present invention further provides a method of treatment comprising measuring the expression of one or more biomarkers of the present invention in a subject suffering from IBD at a first timepoint, administering a therapeutic agent, re-measuring the one or more biomarkers at a second timepoint, comparing the results of the first and second measurements, and optionally modifying the treatment regimen based on the comparison. In one embodiment, the first timepoint is prior to an administration of the therapeutic agent, and the second timepoint is after said administration of the therapeutic agent. In one embodiment, the first timepoint is prior to the administration of the therapeutic agent to the subject for the first time. In one embodiment, the dose (defined as the quantity of therapeutic agent administered at any one administration) is increased or decreased in response to the comparison. In another embodiment, the dosing interval (defined as the time between successive administrations) is increased or decreased in response to the comparison, including total discontinuation of treatment. Dosing interval is inversely related to dosing frequency.

In another aspect, the invention relates to kits for measuring the level of one or more of the biomarkers disclosed herein. Such kits may comprises means for measuring the level of expression of such biomarkers. In some embodiments the kit comprises, e.g., reagents for an ELISA, IHC, FACS®, Western blot, or other immunologically based protein detection method, such as one or more components selected from the group consisting of a capture antibody, a detection (primary) antibody, a secondary antibody, a colorimetric reagent (e.g. an enzyme substrate), a microtiter plate and instructions for use. In other embodiments the detection device is designed to measure biomarker gene expression, such as a chip (or other form of microarray) or a set of primers for use in an amplification-based detection method.

The present invention also provides articles of manufacture including one or more IL-23 antagonists and a document describing the use of the biomarkers of the present invention in monitoring IBD. Specifically, the present invention provides an article of manufacture comprising, packaged and/or sold together, an IL-23 antagonist (or pharmaceutical composition thereof comprising a pharmaceutically acceptable carrier) and a document stating that IBD status in the subject to which the IL-23 antagonist is administered may be monitored by measurement of one or more biomarkers of the present invention, e.g. PAP, LCN2 and CCL20 as measured in serum, or PAP and S100A8/A9 as measured in feces.

In another aspect, the invention relates to methods of selecting subjects, e.g. IBD patients, for treatment with IL-23 antagonists. In one embodiment, the level of IL-23, and/or the level of one, two or more of the biomarkers of the present invention, is determined in a sample from a subject exhibiting signs or symptoms consistent with IBD (e.g. serum, feces or gut biopsy), and the result is used, at least in part, to guide the decision of whether or not to treat with subject with an IL-23 antagonist. An elevated level of IL-23, and/or an elevated level of the one, two or more of the biomarkers of the present invention (compared to reference samples obtained from subjects that do not suffer from IBD) in the sample may suggest that the subject would be a good candidate for treatment with an IL-23 antagonist, such as an anti-IL-23 antibody. In various embodiments, the level of elevation of IL-23, and/or an elevated level of the one, two or more of the biomarkers of the present invention, is 1.5-, 2-, 3-, 5-, 10-, 20-fold or more.

In another aspect, the invention provides methods of treatment of a subject suffering from an inflammatory bowel disease, such as a Crohn's disease patient or an ulcerative colitis patient, in which the therapeutic regimen is modified, at least in part, based on analysis of one or more of the biomarkers of the present invention. In various embodiments, modification of the therapeutic regimen comprises starting treatment, modifying dose, modifying dosing interval, or discontinuing treatment. In various embodiments, treatment comprises administration of an IL-23 antagonist, such as an antagonist antibody that binds specifically to IL-23 (or its subunits) or IL-23 receptor (or its subunits).

### BRIEF DESCRIPTION OF THE DRAWINGS

One-way ANOVA statistical significance is provided for some of the data herein, wherein *=p<0.05, **=p<0.01 and ***=p<0.001. Horizontal lines for datapoints represent the median of observed values. Error bars represent the standard deviation of the mean. Unless otherwise indicated, protein levels were determined by ELISA and gene expression was measured by TaqMan® real time quantitative PCR analysis (and normalized to ubiquitin expression).

FIG. 1 shows CCL20 protein levels in serum samples from Crohn's patients, UC patients, or matched normal controls for each, as further described in Example 5.

FIG. 2 shows results obtained for DMBT1, as further described in Example 6. FIG. 2A shows DMBT1 gene expression in mouse colon tissue from non-disease (naïve), disease (IBD) and IL-23 antagonist treated animals. FIG. 2B shows DMBT1 protein levels (as determined by Western blot) in mouse colon tissue in non-disease, disease and anti-IL-23R treated animals FIG. 2C shows DMBT1 gene expression in human gut biopsy samples from control subjects, active Crohn's patients and Crohn's patients in remission. As with all figures herein presenting data obtained using human biopsy samples, data points represent different biopsy samples, some of which were obtained from the same subject and some of which were obtained from different subjects.

FIG. 3 shows results obtained for MIF, as further described in Example 7. FIG. 3A shows MIF gene expression in mouse colon tissue from non-disease (naïve), disease (IBD) and IL-23 antagonist treated animals. FIG. 3B shows MIF protein levels (determined by Western blot) in mouse colon tissue in non-disease, disease and anti-IL-23R treated animals. FIG. 3C shows MIF protein levels (determined by ELISA) in human serum samples from Crohn's patients, UC patients, or matched normal controls for each.

FIG. 4 shows results obtained for LCN2, as further described in Example 8. FIG. 4A shows a time course of LCN2 protein levels (determined by Western blot) in mouse lamina propria samples from untreated and IL-23 antagonist (anti-IL-23R antibody) treated IBD mice. FIG. 4B shows LCN2 protein levels (determined by ELISA) in human serum samples from Crohn's patients, UC patients, or matched normal controls for each.

FIG. 5 shows results obtained for mouse REG3β and REG3γ, and human PAP/REG3α, as further described in Example 9. FIGS. 5A and 5B show mouse REG3β and REG3γ gene expression, respectively, in mouse colon tissue from non-disease (naïve), disease (IBD) and IL-23 antagonist treated animals. FIG. 5C shows REG3γ protein levels (determined by Western blot) in feces from non-disease, disease and anti-IL-23R treated animals. FIG. 5D shows PAP/REG3α gene expression in human gut biopsy samples from control subjects, active Crohn's patients and Crohn's patients in remission. FIG. 5E shows PAP/REG3α protein levels (determined by ELISA) in human serum samples from Crohn's patients, UC patients, or matched normal controls for each. FIGS. 5F and 5G show time courses of REG3γ protein level (determined by Western blot) from untreated and IL-23 antagonist treated IBD mice, with FIG. 5F reflecting protein levels in mouse colonic epithelial cell samples, and FIG. 5G reflecting protein levels in feces.

FIG. 6 shows results obtained for calprotectin (S100A8/A9), as further described in Example 10. FIGS. 6A and 6B show S100A8 and S100A9 gene expression, respectively, in mouse colon tissue from non-disease (naïve), disease (IBD) and IL-23 antagonist treated animals. FIG. 6C shows S100A8 protein levels (determined by Western blot) in feces from non-disease, disease and anti-IL-23R treated animals. FIGS. 6D and 6E show S100A8 and S100A9 gene expression, respectively, in human gut biopsy samples from control subjects, active Crohn's patients and Crohn's patients in remission. FIG. 6F shows S100A8/A9 complex levels (determined by ELISA) in human serum samples from Crohn's patients, UC patients, or matched normal controls for each.

FIG. 7 shows results obtained for IL-22, as further described in Example 11. FIG. 7A shows IL-22 protein levels in non-disease (pool naïve), disease (IBD) and anti-IL-23R treated animals. FIG. 7B shows a time course of IL-22 protein levels (determined by ELISA) in plasma from untreated and IL-23 antagonist (anti-IL-23R antibody) treated IBD mice. FIG. 7C show IL-22 gene expression in human gut biopsy samples from control subjects, active Crohn's patients and Crohn's patients in remission. FIG. 7D shows IL-22 levels (determined by ELISA) in human serum samples from Crohn's patients, UC patients, or matched normal controls for each.

FIG. 8 shows results obtained for haptoglobin, as further described in Example 12. FIG. 8A shows serum haptoglobin levels (determined by ELISA) in human serum samples from Crohn's patients, UC patients, or matched normal controls for each. FIGS. 8B and 8C show haptoglobin gene expression in mouse colon tissue from non-disease controls (naïve), disease IBD controls (PBS, IgG1 treated), and IL-23 antagonist treated animals (at various doses), wherein anti-IL-23p19 antibody is the IL-23 antagonist in FIG. 8B and anti-IL-23R antibody is the IL-23 antagonist in FIG. 8C. Doses are presented in milligrams-per-kilogram (mpk).

FIG. 9 shows results obtained for GP-39, as further described in Example 22. FIG. 9A shows the level of GP-39 in human serum from Crohn's patients, UC patients, or matched normal controls for each. FIG. 9B shows GP-39 gene expression in mouse colon tissue from non-disease controls (naïve), disease IBD controls (vehicle, IgG1 treated), and anti-IL-23R antibody treated animals at various doses (milligrams-per-kilogram, mpk).

FIG. 10 shows the level of GPX-3 in human serum from Crohn's patients, UC patients, or matched normal controls for each, for example as described further in Example 24.

FIG. 11 shows results obtained for a preventative protocol using a T-cell transfer IBD model in mice. In the preventative protocol, antibodies were added on the same day as T-cell transfer, and weekly thereafter until sacrifice at week 8 post-transfer. FIGS. 11A-11G provide relative RNA expression levels (RU, relative units, relative to ubiquitin) for IL-22 (FIG. 11A), IL-17 (FIG. 11B), TNF-α (FIG. 11C), S100A8/S100A9 (calprotectin) (FIG. 11D), REG3β/REG3γ (FIG. 11E), LCN2 (lipocalin 2) (FIG. 11F), MIF (FIG. 11G), IL-6 (FIG. 11H), lactoferrin (FIG. 11I), GP-39 (FIG. 11J), GPX-2 (FIG. 11K) and neutrophil elastase (FIG. 11L). Data points symbols are as follows: open circles (∘) represent controls where only anti-CD3 T cells were transferred; solid squares (▪) represent animals where Bir14 CD4 T cells were transferred, but which were treated with isotype control antibody; and solid triangles (▴) represent animals where Bir14 CD4 T cells were transferred and which were treated with anti-IL-23p19 antibody. Statistical differences between groups, if significant, are represented by * (p<0.05), ** (p<0.01), or *** (p<0.001). More details are provided in Example 18.

FIG. 12 shows results obtained for a therapeutic protocol using a T-cell transfer IBD model in mice. In the therapeutic protocol, antibodies were added at week 4 post-transfer, and weekly thereafter until sacrifice at week 8 post-transfer. FIGS. 12A-12G provide relative RNA expression levels (RU, relative units, relative to ubiquitin) for IL-22 (FIG. 12A), IL-17 (FIG. 12B), TNF-α (FIG. 12C), S100A8/S100A9 (calprotectin) (FIG. 12D), REG3β/REG3γ (FIG. 12E), LCN2 (lipocalin 2) (FIG. 12F), MIF (FIG. 12G), IL-6 (FIG. 12H), lactoferrin (FIG. 12I), GP-39 (FIG. 12J), GPX-2 (FIG. 12K) and neutrophil elastase (FIG. 12L). Data points symbols and statistical significances are as in FIG. 11, except that open squares (□) represent animals where Bir14 CD4 T cells were transferred and animals were sacrificed at 4 weeks post-transfer without any antibody treatment. More details are provided in Example 18.

### DETAILED DESCRIPTION

As used herein, including the appended claims, the singular forms of words such as “a,” “an,” and “the,” include their corresponding plural references unless the context clearly dictates otherwise. Table 5 below provides a listing of sequence identifiers used in this application. Unless otherwise indicated, or otherwise clear from the context, all proteins, genes and experiments described herein are human proteins and genes, and experiments performed in human systems, respectively.

All references cited herein are incorporated by reference to the same extent as if each individual publication, database entry (e.g. GenBank sequences or GeneID entries), patent application, or patent, was specifically and individually indicated to be incorporated by reference. GenBank accession numbers for nucleic acid and protein sequences referenced herein refer to the contents of the database as of the filing date of this application. Although such database entries may be subsequently modified, GenBank maintains a public record of all prior versions of the sequences as a function of date, making such database entries an unambiguous reference to a specific sequence.

This statement of incorporation by reference is intended by Applicants, pursuant to 37 C.F.R. §1.57(b)(I), to relate to each and every individual publication, database entry (e.g. GenBank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### I. DEFINITIONS

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, may refer to therapeutic treatment, prophylactic or preventative measures, to research and diagnostic applications. For a relapsing/remitting-type disease like IBD, a treatment that prevents, delays or reduces severity of a relapse can be said to either “treat” the overall disease or to prophylactically “prevent” the relapse, and as such the distinction between treatment and prophylaxis is difficult. As use herein, “treatment” refers to reduction of signs or symptoms, or reduction of duration or severity, of an IBD episode active during the start of therapy, whereas “prevention” refers to the prevention, delay or reduction of severity of an IBD episode beginning after the start of therapy, although any given therapeutic regimen may be constitute both treatment and prevention as used herein. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts IL-23 or its receptor (IL-23R/IL-12Rβ1 heterodimer), e.g., in the fluid phase or colloidal phase.

“Disease state” refers to the severity of signs or symptoms of disease, e.g. IBD. A elevated disease state reflects more severe disease, for example in an IBD subject during a flare-up (“flare”). A low disease state reflects less severe or no signs or symptoms, for example in a non-IBD patient or in an IBD patient during remission. “Staging” refers to categorization of the severity of disease in an IBD patient, e.g. for use in guiding treatment. See, e.g., Walfish & Sachar (2007) Inflamm. Bowel Dis. 13:1573.

As used herein, “subject” refers to a specific individual, usually a human, of interest. A “patient” is a human subject who is diagnosed with, or suspected of having, a disease or disorder (e.g. IBD) and/or is under treatment for a disease or disorder.

As used herein, “biological sample” may comprise any sample obtained from a subject, including but not limited to whole blood, plasma, serum, feces (a stool sample), saliva, urine, or biopsy tissue (e.g. colon, small intestine or other gastrointestinal tissue). “Proximal fluid” are fluids close to or in direct contact with disease sites, such as cerebrospinal fluid, synovial fluid, bronchoalveolar lavage, urine, nipple aspirate, feces and colon lavage. Proximal fluids may be more specific to a specific disease process due to their exposure to only a limited number of tissues. Feces, for example, may be a preferred sample for analysis of IBD provided that the biomarker proteins are stable in feces, i.e. that are resistant to proteolytic degradation, since a conventional ELISA may fail to detect proteolytic degradation products. Proteolytic degradation is less of a problem with analytical methods that detect protein fragments directly, such as mass spectrocopic methods.

As used herein, “non-disease levels” refers to the typical or average levels of biomarkers in non-IBD subjects, and “disease levels” refers to the typical or average levels of the biomarkers in IBD patients when not treated and when disease is active. “Reversion,” as used herein, refers to a decrease in biomarker levels toward non-disease levels, whereas “exacerbation” refers to a increase in biomarker levels toward disease levels. A “beneficial response” or “beneficial effect” refers to an improvement in one or more signs or symptoms of a disease or disorder, such as IBD, or other reduction in disease severity. Such beneficial effects are the goal of therapeutic intervention.

Unless otherwise indicated, reference to a “biomarker” relates to form(s) of the polypeptide found naturally in biological samples obtained from humans. For example, proteins having a signal sequence will typically be present in their mature form (lacking the signal peptide). Although a biomarker may be identified by reference to a sequence identifier including the signal sequence, the biomarker that is actually detected in a sample may in fact be the mature (cleaved) form. As used herein, the “level” of a biomarker relates to the amount of the biomarker polypeptide present in a sample, whereas the “expression level” or “level of expression” relates to the amount of mRNA encoding the biomarker. Unless otherwise indicated, “biomarkers of the present invention” refers to the proteins listed in Table 1, although the proteins listed in Tables 3A and 4A may also be referred to as biomarkers depending on the context.

As used herein, the term “antibody” may refer to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

As used herein, when referring to antibodies, the terms “binding fragment thereof” or “antigen binding fragment thereof” encompass a fragment or a derivative of an antibody that still substantially retains the ability to bind to its target. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its affinity for its target, e.g. no more than a 10-fold change in the dissociation equilibrium binding constant (K_{d}). Preferably; a binding fragment or derivative retains at least 25%, 50%, 60%, 70%, 80%, 90%, 95%, 99% or 100% (or more) of its binding affinity, although any binding fragment with sufficient affinity to exert the desired biological effect will be useful. It is also intended that, when specified, a binding fragment can include sequence variants with conservative amino acid substitutions that do not substantially alter its biologic activity.

An “IL-23 antagonist” is a molecule that inhibits the activity of IL-23 in any way. In some embodiments, an antibody or antigen binding fragment thereof of the present invention is an IL-23 antagonist that inhibits IL-23 signaling via the IL-23 receptor, for example by binding to a subunit of IL-23 or its receptor. In other embodiments an IL-23 antagonist is a small molecule or a polynucleotide, such as an antisense nucleic acid or siRNA.

The term “monoclonal antibody”, as used herein, refers to an antibody obtained from a population of substantially homogeneous antibodies, i.e., the individual antibodies comprising the population are identical except for possible naturally occurring mutations that may be present in minor amounts, or glycosylation variants. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific.

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol, 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “b” is added to antibody clone designations when necessary to distinguish humanized antibodies from parental rodent antibodies (although these same designations, depending on the context, may also indicate the human form of a particular protein). The humanized forms of rodent antibodies will generally comprise the same CDR sequences of the parental rodent antibodies, although certain amino acid substitutions may be included to increase affinity, increase stability of the humanized antibody, or for other reasons.

Antibodies also include antibodies with modified (or blocked) Fc regions to provide altered effector functions. See, e.g., U.S. Pat. No. 5,624,821; WO 2003/086310; WO 2005/120571; WO 2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies. A longer half-life may result in less frequent dosing, with the concomitant increased convenience and decreased use of material. See Presta (2005) J. Allergy Clin. Immunol. 116:731 at 734-35.

Antibodies also include antibodies with intact Fc regions that provide full effector functions, e.g. antibodies of human isotype IgG1, which induce complement-dependent cytotoxicity (CDC) or antibody dependent cellular cytotoxicity (ADCC) in the a targeted cell. In some embodiments, the antibodies of the present invention are administered to selectively deplete IL-23R-positive cells from a population of cells. In one embodiment, this depletion of IL-23R-positive cells is the depletion of pathogenic Th17 cells. Depletion of such pathogenic T cell subset may result in sustained remission when effected in subjects suffering from a relapsing/remitting autoimmune disease.

The antibodies of the present invention also include antibodies conjugated to cytotoxic payloads, such as cytotoxic agents or radionuclides. Such antibody conjugates may be used in immunotherapy to selectively target and kill cells expressing IL-23R on their surface. Exemplary cytotoxic agents include ricin, vinca alkaloid, methotrexate, Pseudomonas exotoxin, saporin, diphtheria toxin, cisplatin, doxorubicin, abrin toxin, gelonin and pokeweed antiviral protein. Exemplary radionuclides for use in immunotherapy with the antibodies of the present invention include ¹²⁵I, ¹³¹I, ⁹⁰Y, ⁶⁷Cu, ²¹¹At, ¹⁷⁷Lu, ¹⁴³Pr and ²¹³Bi. See, e.g., U.S. Patent Application Publication No. 2006/0014225.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding compound with a target where the association results in reduction in the normal Brownian motion of the binding compound, in cases where the binding compound can be dissolved or suspended in solution. “Binding composition” refers to a molecule, e.g. a binding compound, in combination with a stabilizer, excipient, salt, buffer, solvent, or additive, capable of binding to a target.

“Effective amount” encompasses an amount sufficient to ameliorate or prevent a symptom or sign of the medical condition. Such an effective amount need not necessarily completely ameliorate or prevent such symptom or sign. Effective amount also means an amount sufficient to allow or facilitate diagnosis. An effective amount for a particular patient or veterinary subject may vary depending on factors such as the condition being treated, the overall health of the patient, the method route and dose of administration and the severity of side effects. See, e.g., U.S. Pat. No. 5,888,530. An effective amount can be the maximal dose or dosing protocol that avoids significant side effects or toxic effects. An effective amount will typically result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject. See, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK.

“Inflammatory disorder” means a disorder or pathological condition where the pathology results, in whole or in part, from, e.g., a change in number, change in rate of migration, or change in activation, of cells of the immune system. Cells of the immune system include, e.g., T cells, B cells, monocytes or macrophages, antigen presenting cells (APCs), dendritic cells, microglia, NK cells, NKT cells, neutrophils, eosinophils, mast cells, or any other cell specifically associated with the immunology, for example, cytokine-producing endothelial or epithelial cells.

An “IL-17-producing cell” means a T cell that is not a classical Th1-type T cell or classical Th2-type T cell, referred to as Th17 cells. Th17 cells are discussed in greater detail at Cua and Kastelein (2006) Nat. Immunol. 7:557-559; Tato and O'Shea (2006) Nature 441:166-168; Iwakura and Ishigame (2006) J. Clin. Invest, 116:1218-1222. “IL-17-producing cell” also means a T cell that expresses a gene or polypeptide of Table 10B of U.S. Patent Application Publication No. 2004/0219150 (e.g., mitogen responsive P-protein; chemokine ligand 2; interleukin-17 (IL-17); transcription factor RAR related; and/or suppressor of cytokine signaling 3), where expression with treatment by an IL-23 agonist is greater than treatment with an IL-12 agonist, where “greater than” is defined as follows. Expression with an IL-23 agonist is ordinarily at least 5-fold greater, typically at least 10-fold greater, more typically at least 15-fold greater, most typically at least 20-fold greater, preferably at least 25-fold greater, and most preferably at least 30-fold greater, than with IL-12 treatment. Expression can be measured, e.g., with treatment of a population of substantially pure IL-17 producing cells. A Th17 response is an immune response in which the activity and/or proliferation of Th17 cells are enhanced, typically coupled with a repressed Th1 response.

Moreover, “IL-17-producing cell” includes a progenitor or precursor cell that is committed, in a pathway of cell development or cell differentiation, to differentiating into an IL-17-producing cell, as defined above. A progenitor or precursor cell to the IL-17 producing cell can be found in a draining lymph node (DLN). Additionally, “IL-17-producing cell” encompasses an IL-17-producing cell, as defined above, that has been, e.g., activated, e.g., by a phorbol ester, ionophore, and/or carcinogen, further differentiated, stored, frozen, desiccated, inactivated, partially degraded, e.g., by apoptosis, proteolysis, or lipid oxidation, or modified, e.g., by recombinant technology.

As used herein, “polymerase chain reaction” or “PCR” refers to a procedure or technique in which minute amounts of a specific piece of nucleic acid, RNA and/or DNA, are amplified as described in, e.g., U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.). As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described. See, e.g., Casset et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos et al. (2002) Curr. Med. Chem. 9:411-420; Monfardini et al. (2002) Curr. Pharm. Des. 8:2185-2199; Domingues et al. (1999) Nat. Struct. Biol. 6:652-656; Sato and Sone (2003) Biochem. J. 371:603-608; U.S. Pat. No. 6,326,482.

“Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. As used herein, an antibody is said to bind specifically to a polypeptide comprising a given sequence if it binds to polypeptides comprising the polypeptide sequence but does not bind to proteins lacking the polypeptide sequence. For example, an antibody that specifically binds to a polypeptide comprising IL-23R may bind to a FLAG®-tagged form of IL-23R but will not bind to other FLAG®-tagged proteins.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ litersimol, as determined, e.g., by Scatchard analysis. Munsen et al. (1980) Analyt. Biochem. 107:220-239.

### II. GENERAL

The present invention provides biomarkers for assessing IBD in a subject, and methods of use of the biomarkers, for example in evaluating the efficacy of a potential treatment method or guiding the course of treatment in an IBD patient. In one embodiment the treatment method comprises administration of an antagonist of the Th17 pathway, such as an antagonist of IL-23 signaling, including but not limited to antibodies that bind to IL-23, IL-23p19, IL-23 receptor, or IL-23R.

Such biomarkers may find use in several contexts. The biomarkers may find use in diagnosing IBD patients or in staging subjects for disease severity. Diagnosis based on biomarker levels requires that a level be selected as the “cut-off” for what is considered disease-positive. This cut-off level may be determined by observing levels in subjects known to have Crohn's disease or ulcerative colitis and levels in control subjects known not to have the diseases. The selected cut-off is the value that best discriminates between disease and non-disease samples. The cut-off may be expressed as an absolute value (e.g. in ng/ml of serum), or more generally as a ratio of disease/control values. Typically the cut-off will be somewhere between the disease and normal levels (if expressed as an absolute value) or a ratio somewhat lower than the disease/control ratio (if expressed as biomarker level ratio). Factors to be considered in selecting the cut-off levels include variability (scatter) in the determinations (both as a result of assay-variability and subject-to-subject variation) and the relative undesirability of false-positive and false-negative results. For example, if there is a desire to be inclusive in diagnosing disease false-negatives are more undesirable than false-positives) a cut-off value can be lower than it would otherwise be, i.e. a lower absolute value (Or lower disease/control ratio) can be considered disease-positive. Such considerations are common in the field of diagnostic testing and are within the skill in the art to resolve.

The biomarkers may also be used to select patient subpopulations likely to respond to treatment with IL-23 antagonists. The present invention demonstrates that the biomarkers disclosed herein are reverted toward non-disease levels when animals are treatment with IL-23 antagonists (e.g., anti-IL-23p19, anti-IL-23R and anti-IL-12/23p40 antibodies), and when humans are treated with TNFα antagonists (e.g., anti-TNFα antibodies). Accordingly, IBD patients having elevated levels of these biomarkers may be considered likely candidates for therapy to revert the levels of the biomarkers to non-disease levels. Conversely, IBD patients without elevated levels of the biomarkers may be poor candidates for treatment with IL-23 antagonists. IBD patients with elevated levels of IL-23 may also be considered likely candidates for treatment with IL-23 antagonists.

Such biomarkers may also find use in subjects undergoing treatment, for example with an IL-23 antagonist, to confirm engagement of the IL-23 pathway, to assess the efficacy of treatment (and modification of therapeutic regimen if necessary), and to monitor patient progress generally. If results demonstrate that a given therapeutic regimen effectively engages the target pathway in a patient, e.g. the IL-23/IL-23 receptor pathway, and yet fails to provide a therapeutic benefit, then it may be that IL-23 signaling is of relatively little practical significance in IBD, at least in the specific patient. Carty & Rampton (2003) Brit. J. Clin. Pharmacol. 56:351.

Such biomarkers may also find use in management of patients in the clinic, for example to inform modification of therapeutic regimen if necessary. A clinician may monitor one or more of the biomarkers of the present invention, using a method of the present invention, to help decide whether dosing should be increased, decreased, or made more or less frequent, depending on the degree to which the patient is responding to existing therapy. Note that reduction of the frequency of administration may constitute a reduced “dose” in that the subject will receive less drug over a given period of time, when the timeframe is longer than a single dosing interval. Measurement of the levels of the biomarkers of the present invention has the advantage that it may be possible to determine which subjects are responding favorably to treatment at an earlier time (i.e. sooner after treatment) than would be possible using standard clinical disease measures, some of which rely at least in part on symptomatic relief. Early discrimination of responders from non-responders allows for earlier modification of dosing or discontinuation. Early modifications of the therapeutic regimen can reduce the time to successful treatment, or reduce the risk of unnecessary exposure to an ineffective drug (with concomitant reduction in expense and side-effects).

Assessment of the efficacy of a given therapeutic regiment for IBD is important for management of patient care, and essential for evaluation of potential therapeutic agents, as in clinical trials. Exemplary measures of IBD include the Crohn's disease activity index (CDAI), the Harvey-Bradshaw “simple index” (SI), the Powell-Tuck index, and the simple colitis clinical activity index (SCCAI). Carty & Rampton (2003) Brit. J. Clin. Pharmacol. 56:351, 354, the disclosure of which is hereby incorporated by reference in its entirety. Such measures often rely, at least in part, on self-reporting of symptoms, which is inherently subjective and non-quantitative. Efficacy of a potential therapeutic agent may be defined as a defined change in one of these indices (e.g. a 70 point drop in CDAI) or achieving remission (defined as CDAI<150). Arnott et al. (2002) Aliment. Pharmacol. Ther. 16:857. More invasive techniques are also used to assess disease progression, such as sigmoidoscopy, colonoscopy, biopsy and ¹¹¹In-labeled neutrophilic granulocyte fecal excretion. Saverumuttu (1986) Digestion 33:74, Roseth et al. (1999) Scand. J. Gastroenterol. 34:50.

Others have proposed a panel of fecal biomarkers, including calprotectin, lactoferrin and polymorphonuclear neutrophil elastase that achieves high diagnostic accuracy for U C. Langhorst et al. (2008) Am. J. Gastroenterol. 103:162. Langhorst et al. propose a comprehensive activity index involving three categories of measurements: i) elevation of two out of the three aforementioned biomarkers above a pre-determined cut-off level in stool, ii) elevated serum levels of C-reactive protein (CRP), and iii) a positive score on either of the CAI (“colitis activity index”) or CDAI. A subject with positive results in any two of the three categories is scored as positive for U C. Langhorst et al. used an “optimized” cut-off of >48 μg/ml for calprotectin. One ELISA kit manufacturer (Immundiagnostik, Bensheim, Germany) suggests that the normal range of calprotectin in stool is <15 ng/ml, and reports a coefficient of variation (CV) of 4-17% between assays. Another ELISA kit manufacturer (HyCult Biotechnology B.V., Uden, The Netherlands) suggests that the normal range of calprotectin in plasma is 0.5-3 μg/ml.

### III. IBD BIOMARKERS

The present invention provides improved biomarkers for IBD that may be measured in readily obtainable biological samples. Potential biomarkers for IBD were obtained by a multi-step process involving comparison of proteins found in human IBD and non-IBD samples (Example 2), detection of candidate biomarkers in an animal model of IBD and determining which biomarker levels change as a function of efficacious treatment with IL-23 inhibitor (Example 3), and further confirming their value as biomarkers in IBD mouse and human disease samples at both the gene expression and protein levels (Example 4). Exemplary biomarkers of the present invention are provided at Table 1.

Other proteins were found to be elevated in whole colon lysates or in colonic epithelial cells from IBD mice, and reverted towards normal upon treatment with anti-IL-23R antibody. These proteins and their presumed human orthologs are provided at Tables 3A and 4A, as described in further detail in Examples 2-4. The human orthologs of these proteins may also serve as biomarkers of IBD, or may serve as potential targets for therapeutic intervention.

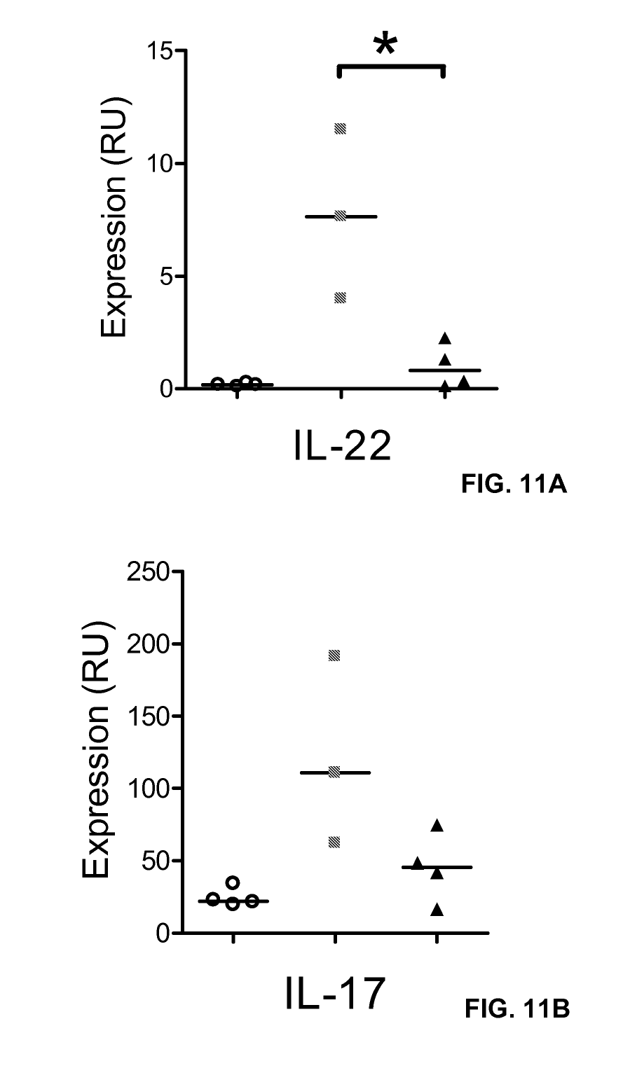

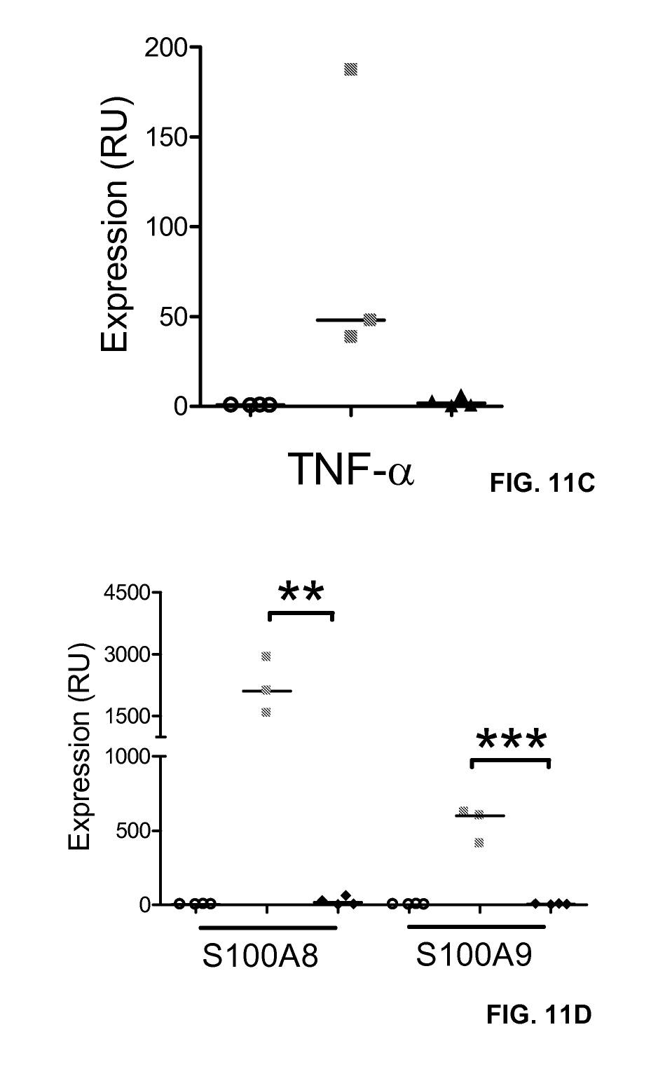

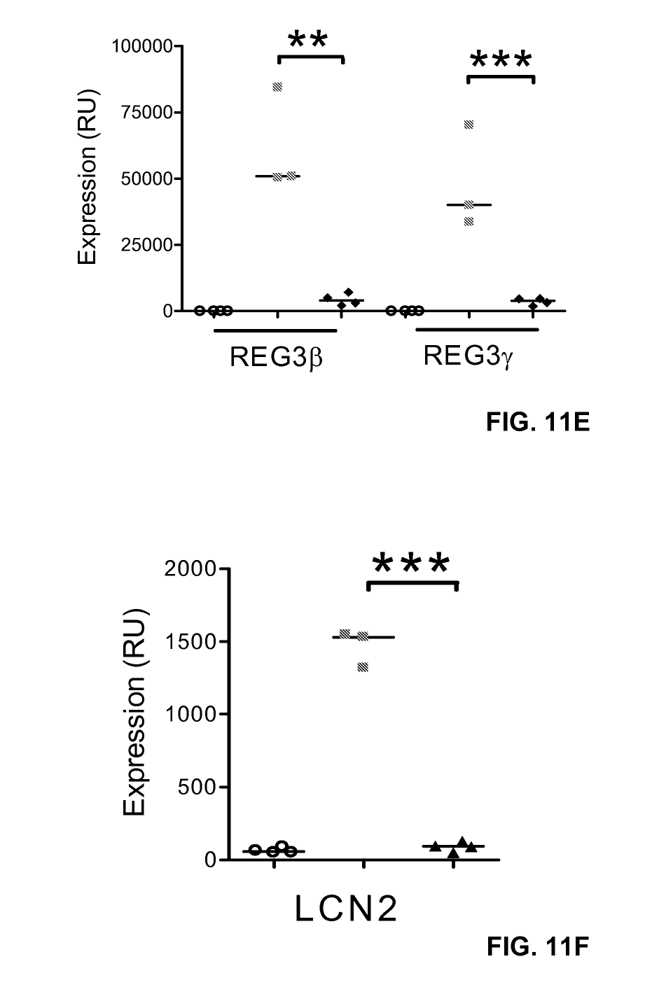

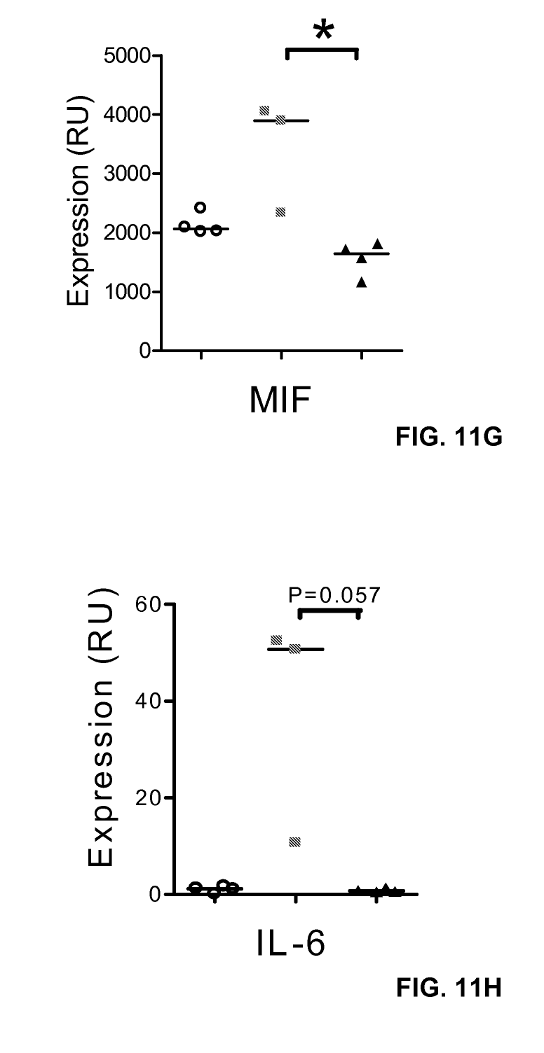

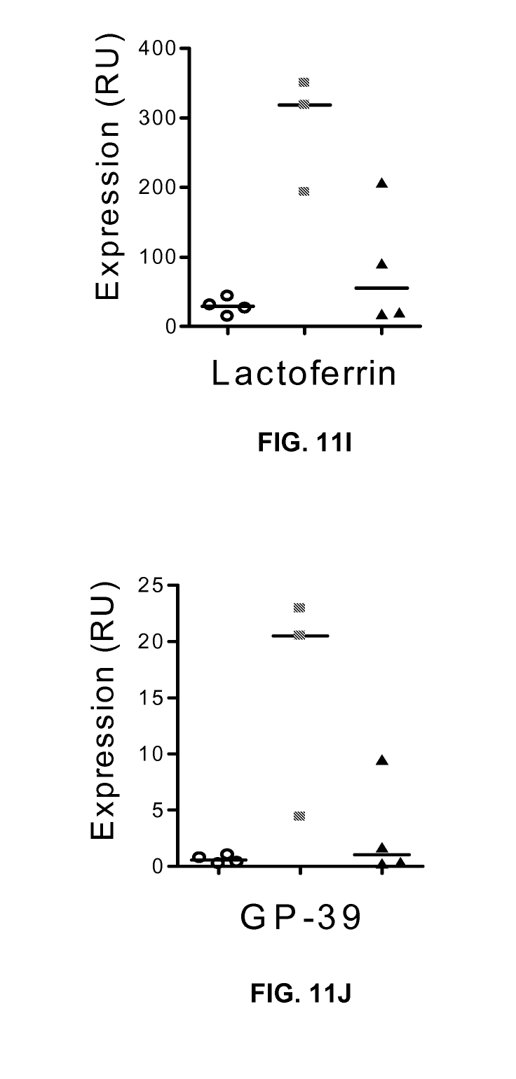

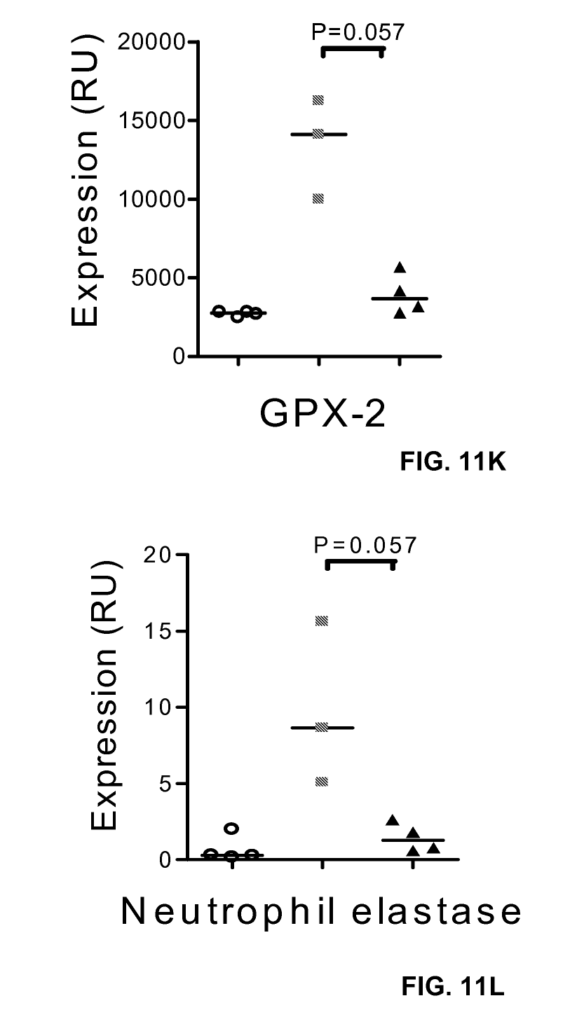

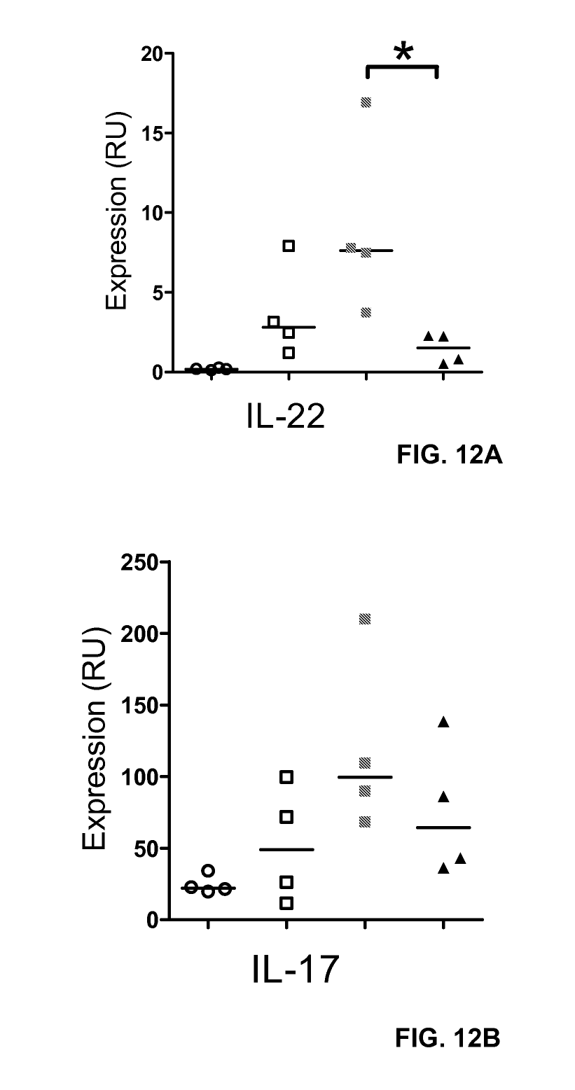

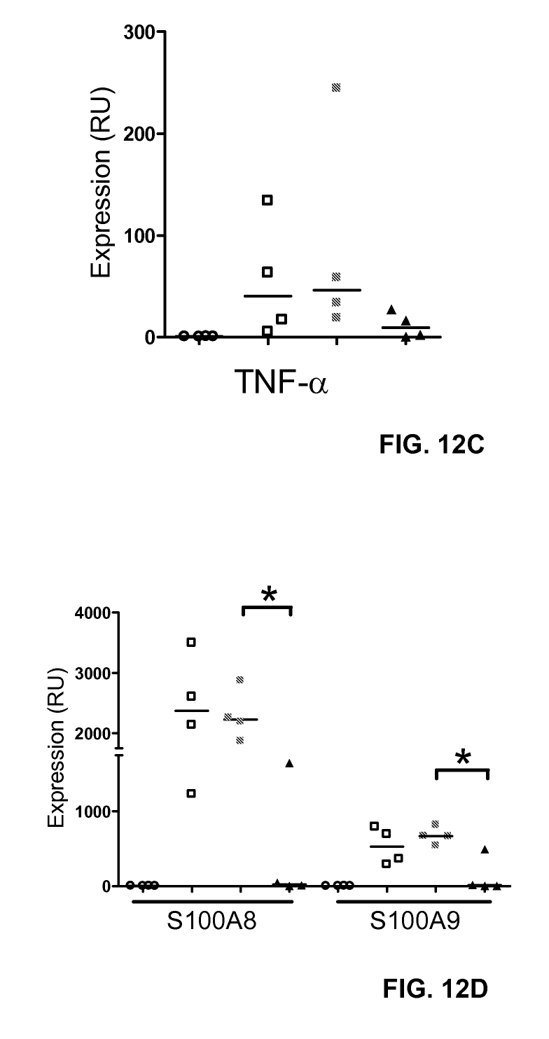

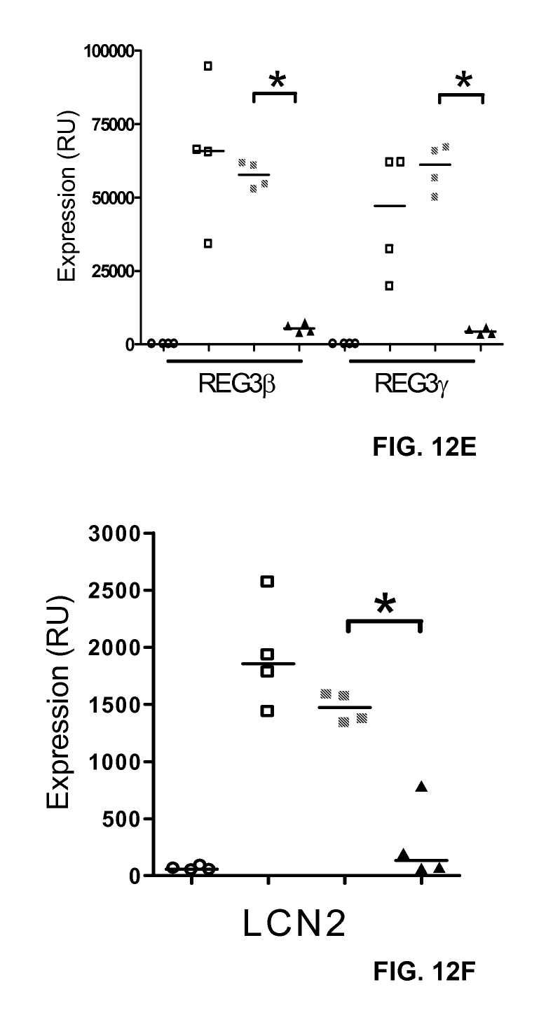

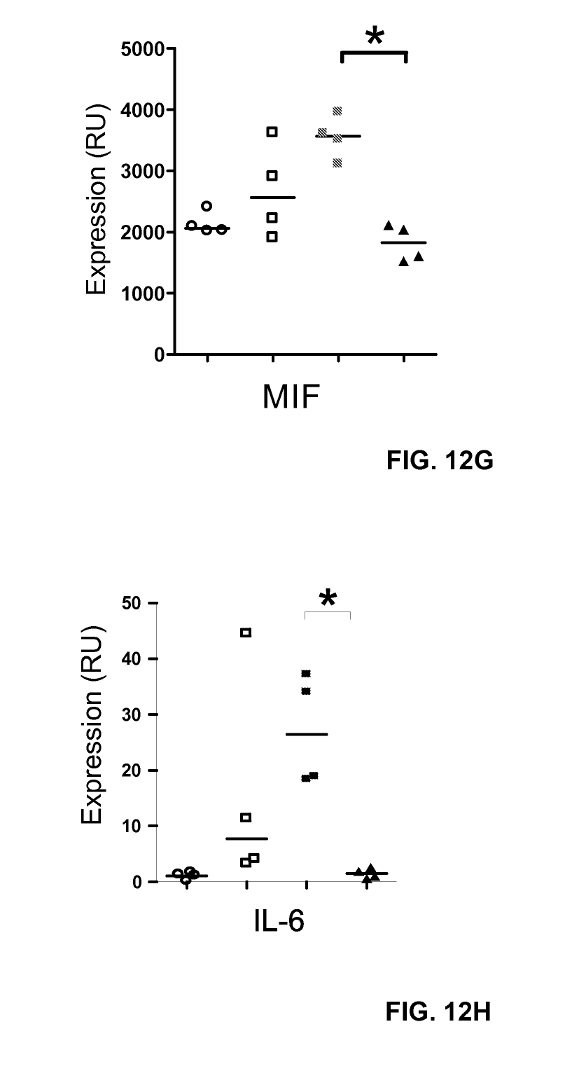

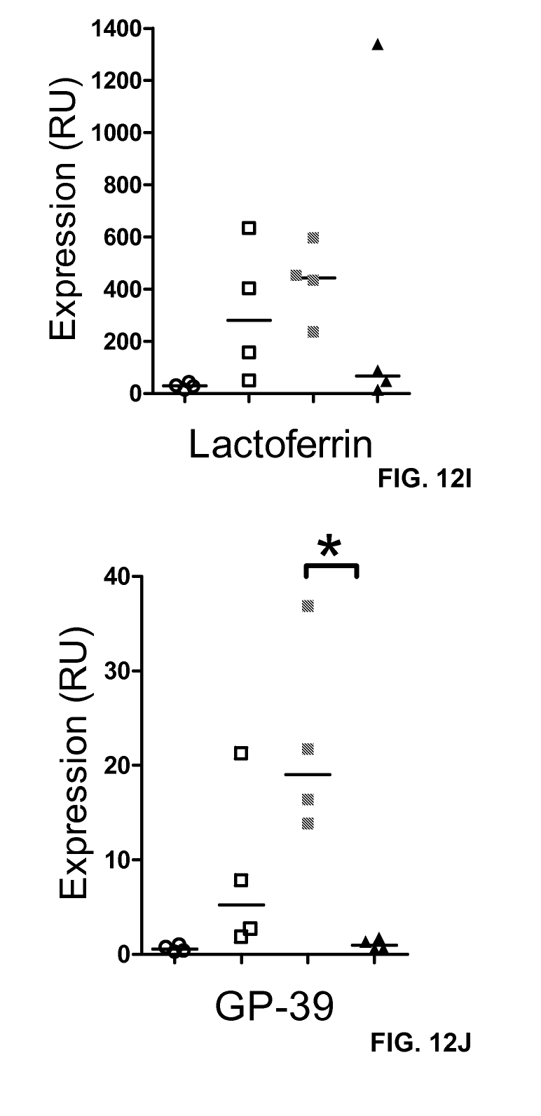

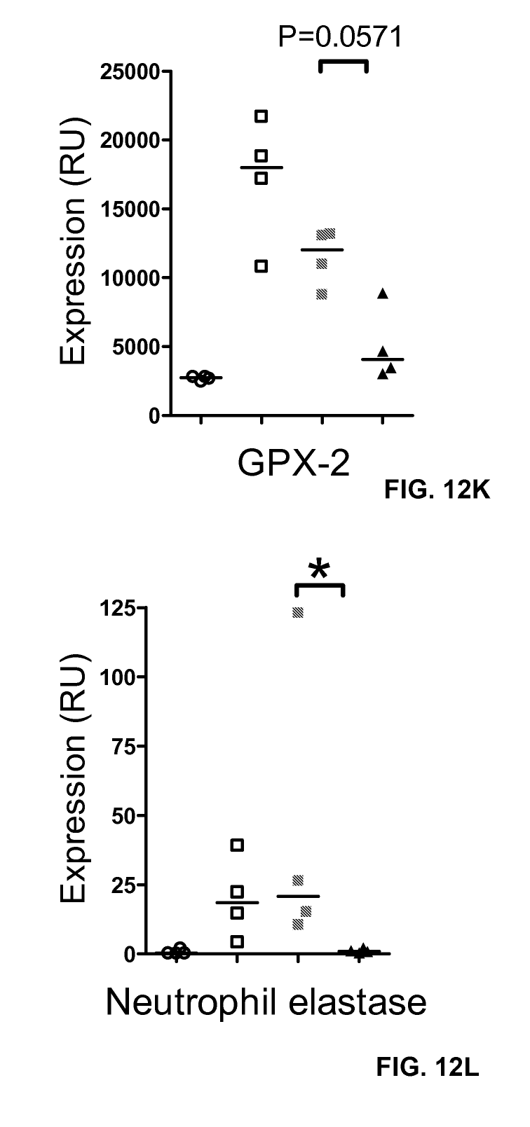

Various biomarkers of the present invention were further studied in a T cell transfer colitis model in mice, using both prophylactic and treatment protocols, as described at Example 18. See also FIGS. 11-12. Unlike the T-cell independent colitis model described in Examples 3 and 4, which primarily reflects the contribution of the innate immune system to colitis, the T cell transfer model necessarily involves the T cell mediated contributions to colitis, and as such represents a distinct model for human disease. In addition, Example 18 includes therapeutic protocols in which anti-IL-23 antibodies were added after the establishment of colitis, rather than earlier in the disease process. Such therapeutic protocol experiments would be expected to better mimic the situation where a patient is not treated until after disease onset. This scenario may represent the most common one with human subjects, who would presumably present for treatment based on the presence of disease symptoms. In contrast, results obtained in a prophylactic model may have particular relevance for preventative treatment of IBD, such as treatment during remission in a relapsing remitting inflammatory bowel disease patient to prevent a flare-up of symptoms.

Pursuant to the present invention, a number of genes have been found to be associated with the disease state of IBD in both animal models and human subjects. Such markers have also been found to correlate with treatment directed at disrupting the Th17 pathway, specifically treatment with anti-IL-23p19 antibodies. Other biomarkers are proposed herein that are not upregulated in mouse IBD models, but instead are found to be upregulated in human disease samples. Still other biomarkers are proposed based on their association with disease pathways.

Table 1 provides information relating to some of these biomarkers. GeneID numbers and GenBank accession numbers are provided, and relate to information available through the United States' National Center for Biotechnology Information (NCBI) site website. All sequences and other information contained within the database entries referenced in Table 1 are hereby expressly incorporated by reference in their entireties.

TABLE 1
IBD Biomarkers

| | | | Nucleic Acid/ | SEQ |
|---|---|---|---|---|
| Gene | GeneID | Aliases | Protein Sequence | ID: |
| CCL20 | 6364 | CKb4; LARC; ST38; MIP3A; | NM_004591.2 | 5 |
| | | MIP-3a; SCYA20 | NP_004582.1 | 6 |
| DMBT1 | 1755 | GP340; muclin; MGC164738 | NM_007329.2 | 7 |
| | | | NP_015568.2 | 8 |
| MIF | 4282 | GIF; GLIF; MMIF | NM_002415.1 | 9 |
| | | | NP_002406.1 | 10 |
| LCN2 | 3934 | NGAL | NM_005564.3 | 11 |
| | | | NP_005555.2 | 12 |
| PAP/ | 5068 | HIP; PAP; PAP1; REG3; | NM_138938.1 | 13 |
| REG3α | REG3A | INGAP; PAP-H; PBCGF; | NP_620355.1 | 14 |
| | | REG-III | | |
| REG3γ | 130120 | PAP1B; PAPIB; UNQ429; | NM_001008387.1 | 15 |
| | REG3G | REG-III; MGC118998; | NP_001008388.1 | 16 |
| | | MGC118999; MGC119001 | | |
| S100A8 | 6279 | P8; MIF; NIF; CAGA; CFAG; | NM_002964.3 | 17 |
| | | CGLA; L1Ag; MRP8; CP-10; | NP_002955.2 | 18 |
| | | MA387; 60B8AG | | |
| S100A9 | 6280 | MIF; NIF; P14; CAGB; CFAG; | NM_002965.3 | 19 |
| | | CGLB; L1AG: LIAG; MRP14; | NP_002956.1 | 20 |
| | | 60B8AG; MAC387 | | |
| IL-22 | 50616 | IL21; TIFa; IL-21; ILTIF; IL- | NM_020525.4 | 21 |
| | IL22 | TIF; IL-D110; zcytol8; | NP_065386.1 | 22 |
| | | MGC79382; MGC79384; | | |
| | | TIFIL-23 | | |
| haptoglobin | 3240 | BP; HPA1S; MGC111141; | NM_005143.3 | 23 |
| | HP | HP2-ALPHA-2 | NP_005134.1 | 24 |
| IL-6 | 3569 | HGF; HSF; BSF2; IL-6; IFNB2 | NM_000600.3 | 25 |
| | IL6 | | NP_000591.1 | 26 |
| IL-17 | 3605 | IL17; CTLA8; IL-17A; IL17-A | NM_002190.2 | 27 |
| | IL17A | | NP_002181.1 | 28 |
| lactoferrin | 4057 | LF; HLF2; GIG12 | NM_002343.2 | 29 |
| | LTF | | NP_002334.2 | 30 |
| GP-39 | 1116 | ASRT7; YKL40; YYL-40; HC- | NM_001276.2 | 31 |
| | CHI3L1 | gp39; HCGP-3P; FLJ38139; | NP_001267.2 | 32 |
| | | DKFZp686N19119; CHI3L1 | | |
| GPX-2 | 2877 | GPRP; GI-GPx; GSHPx-2; | NM_002083.2 | 33 |
| | GPX2 | GSHPX-GI | NP_002074.2 | 34 |
| GPX-3 | 2878 | GPx-P; GSHPx-3; GSHPx-P | NM_002084.3 | 35 |
| | GPX3 | | NP_002075.2 | 36 |
| neutrophil | 1991 | GE; NE; HLE: HNE; ELA2; | NM_001972.2 | 37 |
| elastase | ELANE | PMN-E | NP_001963.1 | 38 |
| TNF-α | 7124 | DIF; TNFA; TNFSF2; TNF- | NM_000594.2 | 39 |
| | TNF | alpha | NP_000585.2 | 40 |
| CRP | 1401 | PTX1; MGC88244; | NM_000567.2 | 41 |
| | | MGC149895 | NP_000558.2 | 42 |

CCL20 (chemokine ligand 20, MIP-3α) is known to play an important role in dendritic cell trafficking and in the recruitment of activated T cells. It is produced by activated Th17 cells (Wilson et al. (2007) Nature Immunol. 8:950), and colonic epithelial cells are known to be a major site of CCL20 production in IBD (Kaser et al. (2004) J. Clin. Immunol. 24:74). CCL20 has also be suggested as a potential susceptibility gene for IBD. Rodriguez-Bores et al. (2007) World J. Gasteroenterol. 13:5560. The signal sequence for CCL20 is 26 amino acids long, meaning that residues 27-96 of SEQ ID NO: 6 correspond to mature CCL20. CCL20 isoform 1 (96 aa) is described herein, whereas isoform 2 (95 aa, NM_{—}001130046.1 and NP_{—}001123518.1) lacks the alanine at position 27, resulting in a mature polypeptide lacking the N-terminal alanine residue of isoform 1.

DMBT1 (Deleted in Malignant Brain Tumors 1) belongs to the scavenger receptor cysteine rich gene family. It is a regulator of the polarization and differentiation of epithelial cells. It also binds and aggregates GRAM-positive and GRAM-negative bacteria. DMBT1 has been associated with Crohn's disease, and as playing a role in intestinal mucosal protection and prevention of inflammation. Rosenstiel et al. (2007) J. Immunol. 178:8203; Renner et al. (2007) Gastroenterol. 133:1499. The signal sequence for DMB is 25 amino acids long, meaning that residues 26-2413 of SEQ ID NO: 8 correspond to mature DMBT1. Transcript variant 2 (isoform b precursor) is described herein, whereas transcript variant 1 (isoform a precursor, NM_{—}004406.2 and NP_{—}004397.2) is missing several exons. An exemplary ELISA for the detection of DMBT1 is provided at Miller et al. (2007) Resp. Res. 8:69.

MIF (Macrophage migration Inhibitory Factor (glycosylation-inhibiting factor)) has been implicated in colitis models in which MIF-deficient mice failed to develop colitis, and established colitis could be treated with anti-MIF immunoglobulins. De Jong et al. (2001) Nature Immunol. 2:1061. MIF does not appear to have an N-terminal signal sequence. Eickhoff et al. (2001) Mol. Med. 7:27.

LCN2 (Lipocalin 2) is an antimicrobial protein expressed in colon epithelial cells and leukocytes in Crohn's disease colon. The antimicrobial function of LCN2 is thought to be mediated by sequestration of iron away from the pathogen. Csillag et al. (2007) Scan. J. Gastroenterol. 42:454. LCN2 has a 20 amino acid signal sequence, meaning that residues 21-198 of SEQ ID NO: 12 correspond to mature LCN2. See Bundgaard et al. (1994) Biochem. Biophys. Res. Comm. 202:1468; WO 2006/091035; U.S. Pat. App. Pub. No. 2005/0261191; EMBL Ace. No. X83006.

Murine REG3β and REG3γ (REGenerating islet-derived 3 beta and gamma) are members of the REG3 family of proteins, members of the C-type lectin family known to participate in cell surface recognition associated with cell growth and differentiation. Human REG3α and REG3γ polypeptides share 85% sequence homology. Human REG3α is also known as Pancreatitis Associated Protein (PAP). REG3α/PAP mRNA is elevated in colonic mucosa from patients with active IBD, and serum levels of PAP were found to correlate with disease severity. Gironella et al. (2005) Gut 54:1244. PAP has been proposed to have anti-inflammatory effects in IBD. Id. It has also been suggested that mouse REG3β and REG3γ may be involved in maintenance of colonic epithelial cell barrier function. Hogan et al. (2006) J. Allergy Clin. Immunol. 118:257; Zheng et al. (2008) Nature Medicine 14:282. PAP transcript variant 2 is reported herein, but it is also found as variant 1 (NM_{—}002580.1 and NP_{—}002571.1) and variant 3 (NM_{—}138937.1 and NP_{—}620354.1). PAP has a 26 amino acid signal sequence, meaning that residues 27-175 of SEQ ID NO: 14 correspond to mature PAP. Human REG3γtranscript variant 1 is reported herein, but a second transcript variant encoding the same polypeptide is disclosed at GenBank Ace. No, NM_{—}198448.2. Human REG-3γ has a putative 26 amino acid signal sequence, meaning that residues 27-175 of SEQ ID NO: 16 correspond to mature human REG3γ.

Calprotectin (S100A8/A9) is a calcium binding protein produced by neutrophils, monocytes, and epithelial cells under inflammatory conditions, Foell et al. (2007) J. Leukocyte Biol. 81:28. It can inhibit the growth of Staphylococcus aureus in abscesses by chelation of nutrient Mn⁺⁺ and Zn⁺⁺. Corbin et al. (2008) Science 319:962. Neither S100A8 or S100A9 has a classical signal sequence. Rammes et al. (1997) J. Bio. Chem. 272:9496. S100A8 (S100 calcium-binding protein A8) has also been known as calgranulin A, cystic fibrosis antigen, myeloid-related protein 8 and granulocyte L1 protein. S100A9 (S100 calcium-binding protein A9) has also been known as calgranulin B, cystic fibrosis antigen B and myeloid-related protein 14. Nomenclature relating to calprotectin is discussed at Fagerhol (1996) J. Clin. Pathol. 49:M74.

Calprotectin has long been associated with intestinal inflammation, and has been proposed as a marker for IBD. See, e.g., Lugering et al. (1995) Digestion 56:406; von Roon et al. (2007) Am. J. Gastroenterol. 102:803; Leach et al. (2007) Scand. J. Gastroenterol. 42:1321; Langhorst et al. (2008) Am. J. Gastroenterol. 103:162. Its measurement in feces has been shown to be useful in detecting active IBD and predicting recurrence of disease. Angriman et al. (2007) Clinica Chimica Acta 381:63. In 2006, the U.S. Food & Drug Administration (FDA) approved the PhiCal® Fecal Calprotectin Immunoassay (Genova Diagnostics, Inc., Asheville, N. Carolina, USA; Calpro AS, Oslo, Norway) for use in diagnosing IBD. See U.S. Pat. Nos. 4,833,074; 5,455,160; 6,225,072; FDA New Device Clearance K050007 (Apr. 26, 2006). The test involves an ELISA assay with colorimetric readout. Human calprotectin detection kits are also commercially available from, e.g., Immunodianostik AG, Bensheim, Germany and Cell Sciences', Canton, Mass., USA. Fecal calprotectin assay provides a non-invasive method to measure intestinal inflammation even when sub-clinical. Arnott et al. (2002) Aliment. Pharmacol. Ther. 16:857. See also U.S. Pat. No. 6,225,072. Detection of fecal calprotectin at levels greater than 100 μg/g of stool is diagnostic for IBD. von Roon et al. (2007) Am. J. Gastroenterol. 102:803. According to literature provided with the PhiCal® Test (Calpro A S, Oslo, Norway), fecal calprotectin levels of over 50 μg/g are regarded as a “positive” result, based on median fecal calprotectin levels of over 1700 μg/g (e.g. 200-20.000 μg) in IBD patients and 25 μg/g normal healthy subjects.

IL-22 (interleukin 22) is a pro-inflammatory cytokine known to be expressed from Th17 cells. Wilson et al. (2007) Nature Immunol. 8:950. Increased serum levels of IL-22 have also been associated with IBD. Schmechel et al. (2008) Inflamm. Bowel Dis. 14:204; Brand et al. (2006) Am. J. Physiol. Gastrointest. Liver Physiol. 290:G827-G838. IL-22 has been shown to induce haptoglobin expression in hepatic cells (Dumoutier at al. (2000) Proc. Nat'l. Acad. Sci. (USA) 97:10144), to upregulate expression of REG3α, REG3γ, S100A8, S100A9 and haptoglobin in colon epithelial cells (Zheng et al. (2008) Nature Medicine 14:282), and to upregulate expression of Si 00A8 and S100A9 in keratinocytes (Boniface et al. (2005) J. Immunol. 174:3695). IL-22 has a 33 amino acid signal sequence, meaning that residues 34-179 of SEQ ID NO: 22 correspond to mature IL-22. Xie et al. (2000) J. Biol. Chem. 275:31335.

Haptoglobin is a tetramer of two alpha and two beta chains. The disclosed sequence (NP_{—}005134.1, SEQ ID NO: 24) is a preproprotein that is processed to yield the alpha and beta chains. Haptoglobin binds to free plasma hemoglobin, which allows degradative enzymes to gain access to the hemoglobin, while at the same time preventing loss of iron through the kidneys and protecting the kidneys from damage by hemoglobin. Haptoglobin has been linked, inter alia, to diabetic nephropathy, coronary artery disease in type 1 diabetes and Crohn's disease. Haptoglobin has also been reported to be elevated in Crohn's disease and ulcerative colitis patients. Vucelic et al. (1991) Acta Med. Austriaca 18:100 (abstract). IL-22 is known to induce haptoglobin expression in hepatic cells. Dumoutier et al. (2000) Proc. Nat'l. Acad. Sci. (USA) 97:10144. Haptoglobin has a 18 amino acid signal sequence, meaning that residues 19-347 of SEQ ID NO: 24 correspond to mature haptoglobin. Haptoglobin transcript variant 1 is described herein, but transcript variant 2, lacking two exons, is disclosed at GenBank Acc. Nos. NM_{—}001126102.1 and NP_{—}001119574.1. Clinical diagnostic assays for human haptoglobin include the Haptoglobin SPQ II kit (DiaSorin S.p.A., Vercelli, Italy), Tina-Quant® Haptoglobin kit (Roche Diagnostics Corp., Indianapolis, Ind., USA), NOR-Partigen® Immunodiffusion Plates for Haptoglobin (Siemens, Dade Behring, Eschborn, Germany), K-Assay for Haptoglobin (Kamiya Biomedical Co., Seattle, Wash., USA), and the MININEPH benchtop laser nephelometer Haptoglobin assay (The Binding Site, Birmingham, UK).

IL-6 (interleukin-6) is a pro-inflammatory cytokine that is expressed as a 212 amino acid precursor, from which a 29 amino acid signal peptide is cleaved to generate the 183 amino acid mature IL-6 polypeptide. IL-6 plays a role in inflammation, and in the maturation of B cells. It is typically produced at a site of acute or chronic inflammation, where it is secreted into the serum. Elevated IL-6 has been associated with poor prognosis for some cancers. IL-6 antagonists (e.g. tocilizumab) have been proposed for treatment of a number of inflammatory disorders (Nishimoto & Kishimoto (2008) Handb. Exp. Pharmacol. 181:151) and cancers.

IL-17 (interleukin-17, CTLA-8) is a pro-inflammatory cytokine that is expressed as a 155 amino acid precursor, from which a 23 amino acid signal peptide is cleaved to generate the 132 amino acid mature IL-17 polypeptide, IL-17 is the characteristic cytokine produced by the Th17 T cell subset. Th17 cells have been associated with a number of autoimmune and inflammatory disorders, including inflammatory bowel disease. Fouser et al. (2008) Immunol. Rev. 226:87. IL-23 is a key cytokine contributing to the development and maintenance of Th17 cells, and a mutation in the gene for its receptor (IL-23R) was associated with inflammatory bowel disease in a genome-aide association study. Duerr et al. (2006) Science 314:1461. High levels of IL-17 are associated with several chronic inflammatory diseases, such as rheumatoid arthritis, psoriasis and multiple sclerosis.

Lactoferrin is expressed as a 710 amino acid precursor, from which a 19 amino acid signal sequence is cleaved. Lactoferrin is found in the secondary granules of neutrophils. The protein is a major iron-binding protein and is believed to be involved in regulation of iron homeostasis, host defense against a broad range of microbial infections, anti-inflammatory activity, regulation of cellular growth and differentiation and protection against cancer development and metastasis. Fecal lactoferrin has been proposed as a biomarker in monitoring treatment of IBD with the anti-TNF-α antibody infliximab. Buderus et al. (2004) Digestive Diseases and Sciences 49: 1036.

GP-39 (cartilage glycoprotein-39, YKL-40) is a 383 amino acid chitinase-like protein involved in inflammation and tissue remodeling. It is produced as a 383 amino acid polypeptide from which a 21 amino acid signal sequence is cleaved. GP-39 has been proposed as a serum biomarker for asthma (Ober et al. (2008) N. Engl. J. Med. 358:1725) and as a biomarker for various cancers (Roslind & Johansen (2008) Methods in Molecular Biology 511:159).

GPX-2 (glutathione peroxidase 2) is a selenium-dependent glutathione peroxidase that is detected mainly in gastrointestinal tissues. Position 40 of the 190 amino acid polypeptide chain is a selenocysteine encoded by TGA, which is normally a termination codon. This residue is involved in the catalytic activity of the protein, i.e. reduction of reactive oxygen species by reduced glutathione.

GPX-3 (glutathione peroxidase 3) is a selenium-dependent glutathione peroxidase. It has a 20 amino acid signal peptide, cleavage of which leaves a 206 amino acid soluble mature form. Position 73 of the 226 amino acid polypeptide chain is a selenocysteine encoded by TGA, which is normally a termination codon, but which is read to encode an amino acid due to a stem-loop structure in the 3′ untranslated region of the message. Residue 73 is involved in the catalytic activity of the protein, i.e. reduction of reactive oxygen species by reduced glutathione.

Neutrophil elastase is a serine protease predominantly secreted by neutrophils. It is expressed as a 267 amino acid polypeptide, of which the first 29 amino acids are a signal sequence. It causes severe tissue damage when secreted at a site of inflammation. Although key to host defense against some bacterial pathogens, neutrophil elastase has been implicated in the pathology of pulmonary emphysema, arthritis, nephritis and certain skin diseases. Plasma from healthy people contains approximately 55 ng/ml (29-86 ng/ml) of neutrophil elastase. Product Data Sheet for ELISA Kit for Human Neutrophil Elastase, HyCult Biotechnology B.V. (Uden, The Netherlands).

TNF-α (tumor necrosis factor alpha) is a pro-inflammatory cytokine with a role in host defense and inflammatory responses. It is produced as a 233 amino acid precursor, which is cleaved to remove a 76 amino acid N-terminal portion, leaving residues 77-233 as the mature soluble form. See SEQ ID NO: 40 (in which sequence numbering is with respsect to the mature, as opposed to the precursor, form). Elevated TNF-α has been associated with a number of autoimmune diseases, including asthma, type-2 diabetes, Crohn's disease and rheumatoid arthritis. See Chatzantoni & Mouzaki (2006) Curr. Top. Med. Chem. 6:1707. TNF-α inhibitors, such as anti-TNF-α antibodies (infliximab, adalimumab) and soluble receptor (etanercept) have been approved for the treatment of a number of autoimmune and inflammatory disorders, including Crohn's disease, rheumatoid arthritis, ankylosing spondylitis and psoriatic arthritis. Nash & Florin (2005) Med. J. Aust. 183:205.

CRP (C-reactive protein) is a 224 amino acid polypeptide with an 18 amino acid signal sequence, leaving a 206 amino acid mature form. CRP has long been recognized as an acute phase reactant that rises dramatically in concentration after tissue injury or inflammation. Elevated CRP levels have been associated with coronary artery disease. CRP has long been used as a marker for inflammatory bowel disease, especially Crohn's disease. See, e.g., Vermerie et al. (2004) Inflamm. Bowel Dis. 10:661 and Keshet et al. (2009) Am. J. Med. Sci. 337:248. For use as a biomarker in humans, CRP levels may be determined using a custom-designed ELISA assay, or, for example, using a commercially available ELISA kit, such as the Quantikine® human CRP Immunoassay kit (catalog DCRPOO) from R&D Systems® (Minneapolis, Minn., USA) and the AssayMax human lactoferrin ELISA kit (catalog EL2001-1) from AssayPro (St. Charles, Mo., USA).

The ratios of the levels of various biomarkers of the present invention were calculated for samples obtained from disease versus non-disease subjects (human or mouse), and are provided at Table 2. Results for S100A8/S100A9 for human serum are combined because the calprotectin ELISA that was used detects the S100A8/S100A9 complex, rather than the individual subunits.

TABLE 2
IBD Biomarker Protein Level Ratios

| | | | Ratio: | Ratio: | |
|---|---|---|---|---|---|
| | Ratio: | Ratio: | Disease/Anti- | Disease/Anti- | Ratio: |
| | Disease/Naïve | Disease/Naïve | IL-23R | IL-23R | Disease/Control |
| Biomarker | mouse colon | mouse feces | mouse colon | mouse feces | human serum |
| DMBT1 | 20.4 | — | 3.8 | — | — |
| MIF | 4.8 | — | 2.7 | — | 5.7 |
| PAP | — | — | — | — | 2.4 |
| REG3γ | — | 5.5 | — | 3.2 | — |
| S100A8 | — | 17.5 | — | 8.8 | 1.7 |
| S100A9 | — | — | — | — | |

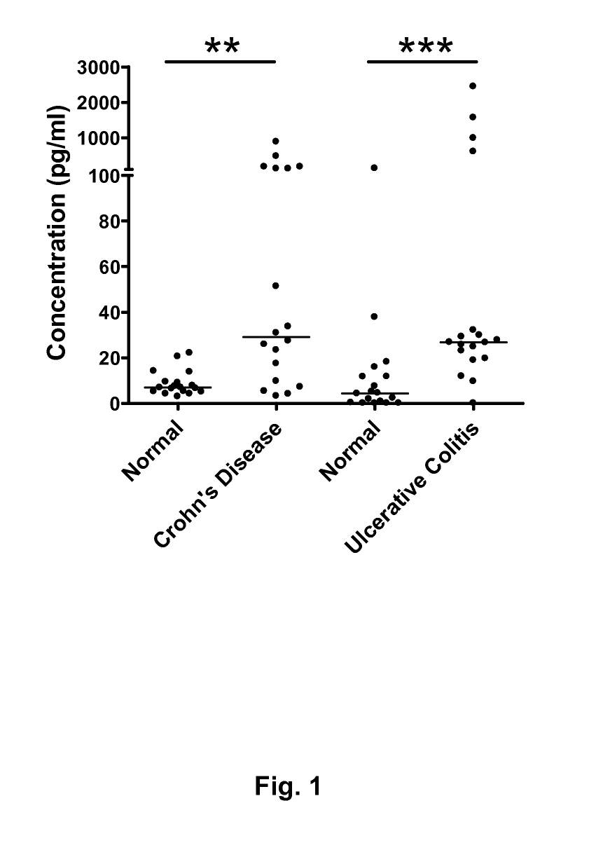

The ratios provided in Table 2 can be used to estimate the level of expression of a given biomarker that corresponds to active IBD. In addition to the ratios in Table 2, FIGS. 1-12 also provide quantitative values for the difference in level for various biomarkers of the present invention, either at the protein or gene expression level. Inspection of these data may also guide selection of an appropriate “cut-off” level for deciding what constitutes active IBD. See, e.g., Examples 5-12 and 19-26. More robust conclusions may be drawn, of course, when the level of the biomarkers disclosed herein is determined in human subjects, for example as in some of the figures provided herein, or in the course of a clinical trial.

The above-referenced biomarkers may be used in any permutation or combination to predict or monitor IBD. In one embodiment S100A8/A9 and PAP are measured in stool samples to monitor or predict IBD. In another embodiment, any two or more of S100A8/A9, MIF, PAP, LCN2, CCL20, IL-22 and haptoglobin are measured in serum samples to monitor or predict IBD. In one specific embodiment, the combination comprises IL-22, PAP and S100A8/A9. In another specific embodiment, the combination comprises IL-22 and S100A8/A9. In yet another specific embodiment, the combination comprises PAP, LCN2 and CCL20.

### IV. IL-23 ANTAGONISTS

IBD may be treated using antagonists of IL-23. Antagonists of IL-23 include agents, such as antibodies or fragments thereof, that bind to IL-23, comprising p19 and p40 subunits. The sequence of human IL-23p19 (GeneID 51561, IL23A) is found at GenBank Accession No: NP_{—}057668.1 (SEQ ID NO: 1) and the sequence of human IL-12/IL-23 p40 (GeneID 3593, IL12B) is found at GenBank Accession No. P29460 (SEQ ID NO: 2). The mature form of p19 (with the 19 amino acid signal sequence removed) comprises residues 20-189 of SEQ ID NO: 1. IL-23p19 has also been known as IL-B30, SGRF and MGC79388. The mature form of p40 (with the 22 amino acid signal sequence removed) comprises residues 23-328 of SEQ ID NO: 2. IL-12/23p40 has also been known as CLMF, NKSF, CLMF2 and NKSF2.

Antagonists of IL-23 also include agents, such as antibodies or fragments thereof, that inhibit IL-23 signaling via the IL-23 receptor, comprising IL-23R and IL-121431 subunits. The amino acid sequence for human IL-23R (GeneID 149233, IL23R) is found at GenBank Accession No: NP_{—}653302.2 (SEQ ID NO: 3). The mature form of IL-23R (with the 23 amino acid signal sequence removed) comprises residues 24-629 of SEQ ID NO: 3. The amino acid sequence for human IL-12Rβ1 (GeneID 3594, IL-12Rβ1) is found at GenBank Accession Nos: NP_{—}005526.1 (SEQ ID NO: 4). The mature form of IL-12 Rβ1 (with the 24 amino acid signal sequence removed) comprises residues 25-662 of SEQ ID NO: 4.

The IL-23 antagonists of the present invention can also be used in combination with one or more antagonists of other cytokines (e.g. antibodies), including but not limited to, IL-17A, IL-17F, TNF-α, IL-1β, IL-6 and TGF-β. See, e.g., Veldhoen (2006) Immunity 24:179-189; Dong (2006) Nat. Rev. Immunol. 6(4):329-333.

In one embodiment, the anti-human IL-23p19 antibody comprises the heavy and light chain variable domains of humanized antibody hu13B8, as disclosed in commonly assigned International Pat. Appl. Pub. No. WO 2008/103432. In another embodiment the anti-human IL-23p19 antibody comprises one, two, three, four, five or six of the CDR sequences of antibody hu13B8. In another embodiment the anti-human IL-23p19 antibody competes with antibody hu13B8 for binding to human IL-23. In another embodiment the anti-human IL-23p19 antibody binds to the same epitope on human IL-23 as hu13B8. In other embodiments, the anti-human IL-23p19 antibody is able to block binding of human IL-23p19 to the antibody produced by the hybridoma deposited with ATCC under accession number PTA-7803 in a cress-blocking assay. In yet further embodiments, the anti-human IL-23p19 antibody hinds to the same epitope as the antibody produced by the hybridoma deposited with ATCC under accession number PTA-7803. A hybridomas expressing antibody 13B8 (mouse IgG 1 kappa) was deposited pursuant to the Budapest Treaty with American Type Culture Collection (ATCC—Manassas, Va., USA) on Aug. 17, 2006 under Accession Number PTA-7803.

In another embodiment, the anti-human IL-23R antibody comprises the heavy and light chain variable domains of humanized antibodies hu20D7 (variant-a, -b or -c) or hu8B10 (or variants-b or -c), as disclosed in commonly assigned International Pat, Appl. Pub. No. WO 2008/106134, U.S. Provisional Patent Application Nos. 61/092,312 (filed Aug. 27, 2008) and 61/223,971 (filed Jul. 8, 2009). In another embodiment the anti-human IL-23R antibody comprises one, two, three, four, five or six of the CDR sequences antibody hu20D7 (or variants thereof) or hu8B10 (or variants thereof). In another embodiment the anti-human IL-23R antibody competes with antibody hu20D7 or hu8B10 for binding to human IL-23R. In another embodiment the anti-human IL-23R antibody binds to the same epitope on human IL-23 as hu20D7 or hu8B10. In other embodiments, the anti-human IL-23R antibody is able to block binding of human IL-23R to the antibody produced by the hybridoma deposited with ATCC under accession number PTA-7800, and/or the antibody produced by the hybridoma deposited under ATCC accession number PTA-7801, in a cross-blocking assay. In yet further embodiments, the anti-human IL-23R antibody binds to the same epitope as the antibody produced by the hybridoma deposited with ATCC under accession number PTA-7800 or the antibody produced by the hybridoma deposited under ATCC accession number PTA-7801. Hybridomas expressing antibodies 8B10 (rat IgG2a kappa) and 20D7 (mouse IgG1 kappa) were deposited pursuant to the Budapest Treaty with American Type Culture Collection (ATCC—Manassas, Va., USA) on Aug. 17, 2006 under Accession Numbers PTA-7800 and PTA-7801, respectively.

Exemplary anti-IL-23p19 antibodies are disclosed, e.g., in commonly assigned U.S. Pat. Appl. Pub. No. 2007/0048315 (to Schering Corporation, disclosing anti-IL-23p19 antibodies) and International Pat. Appl. Pub. No. WO 2008/103473. Additional exemplary anti-IL-23 antibodies are disclosed, e.g., in U.S. Pat. No. 7,247,711 (to Centocor, disclosing anti-IL-23p40-specific antibodies), U.S. Pat. No. 7,491,391 and U.S. Pat. Appl. Pub. No. 2007/0218064 (to Centocor, disclosing anti-IL-23p19 antibodies), International Pat. Appl. Pub. No. WO 2007/024846 (to Eli Lilly, disclosing anti-IL-23p19 antibodies), U.S. Pat. Appl. Pub. No. 2009/0123479 (to Glaxo SmithKline, disclosing anti-IL-23p19 antibodies), International Pat. Appl. Pub, No, WO 2009/068627 (to Ablynx, disclosing anti-IL-23 nanobodies) and U.S. Pat. Appl. Pub. No. 2008/0095775 (to Zymogenetics, disclosing a bispecific antibody to IL-23p19 and IL-17). Exemplary IL-12/IL-23 (anti-p40) antibodies already in clinical trials include the Centocor's fully human antibody ustekinumab (CNTO 1275) and Abbott's fully human antibody briakinumab (ABT-874).

In various embodiments the IL-23 antagonists of the present invention comprise antigen binding fragments of antibodies, such as fragments of any of the IL-23 antagonist antibodies referred to herein. Such fragments include, but are not limited to, Fab, Fab′, Fab′-SH, Fv, scFv, F(ab′)₂, nanobody and a diabody.

Other IL-23 antagonists include those disclosed in U.S. Pat. Appl. Pub. No. 2007/066550 (to Archemix, disclosing anti-IL-12/23 aptamers). An exemplary IL-12/IL-23 small molecule inhibitor already in clinical trials is Synta Phamarceuticals' STA-5326.

### V. DETERMINATION OF EXPRESSION LEVELS

In one aspect, the invention involves determining whether a sample from a subject exhibits increased or decreased levels of one or more biomarkers compared with control levels. Biomarker levels can be quantitated by any method known in the art, including but not limited to, mass spectrometry, Western blot, IBC or ELISA. Means for determining the level of the biomarker(s) of the present invention include, but are not limited to, the methods disclosed herein, and their equivalents. Comparison of biomarker levels may be used, e.g., to diagnose IBD (comparing a sample from a subject suspected of having IBD to samples from non-IBD subjects), differentially diagnose Crohn's disease and ulcerative colitis, stage IBD patients, select patients for treatment with an IL-23 antagonist, evaluate disease status (comparing samples taken from subjects during flare or in remission) or evaluate the effectiveness of therapy (comparing samples obtained before and after treatment). Determination of the level of biomarkers at the protein level, as opposed to at the gene expression level, is convenient because protein can be detected non-invasively, such as in plasma and feces.

In one embodiment, biomarker protein levels are determined by Western blot (immunoblot), for example as follows. A biological sample is electrophoresed on 10% sodium dodecyl sulfate polyacrylamide gel (SDS-PAGE) and transferred (e.g. electroblotted) onto nitrocellulose or polyvinylidene fluoride (PVDF) some other suitable membrane. The membrane is then incubated with a primary antibody that binds to the biomarker protein being evaluated, washed, optionally incubated with a detectably labeled secondary antibody that binds to the primary antibody, and optionally washed again. The presence of the secondary antibody is then detected (or primary antibody if it is detectably labeled), for example by radioactivity, fluorescence, luminescence, enzymatic activity (e.g. alkaline phosphatase or horseradish peroxidase) or other detection or visualization technique known to those of skill in the art. In one embodiment, the detectable label is used to produce an autoradiograph, which is scanned and analyzed. In other embodiments, the gel is imaged directly without the use of an autoradiograph. Observed biomarker band intensity may optionally be normalized to a control protein present in the sample, such as actin or tubulin.

In yet another embodiment, biomarker levels are determined by ELISA. In one embodiment, the sandwich ELISA, a first antibody specific for the biomarker of interest (the “capture antibody”) is coated in the well of a plate (e.g. a 96-well microtiter plate), and the plate is then blocked with, e.g., bovine serum albumin (BSA) or casein. Standards or samples are pipetted into the wells so that biomarker polypeptide present in the samples can bind to the immobilized antibody. The wells are washed and a (second) biotinylated anti-biomarker antibody is added. This second anti-biomarker antibody must be able to bind to the biomarker even while the biomarker is bound to the first antibody. In other embodiments, the second antibody is the same as the first antibody, for example if the biomarker forms a multimer. In some embodiments the second antibody is a distinct, non-crossreacting antibody. In yet other embodiments the second antibody binds to an entirely separate polypeptide chain, for example when the biomarker to be detected is present as a heterodimeric complex (e.g. calprotectin). After washing away unbound biotinylated antibody, HRP-conjugated streptavidin (or some functionally equivalent detection reagent) is pipetted to the wells. Alternatively, the biotinylated antibody can be replaced with an antibody having a directly detectable label, obviating the need for the streptavidivn step. The wells are again washed, a TMB substrate solution is added to the wells, and color develops in proportion to the amount of biomarker bound. Stop solution is added to the reaction, which changes the color from blue to yellow, and the intensity of the color is measured at 450 nm. See e.g., Human IGF-BP-2 ELISA Kit from RayBiotech, Inc.; Norcross, Ga., USA; and Angervo et al., (1992) Biochem. Biophys. Res. Comm. 189: 1177; Kratz et al. (1992) Exp. Cell Res. 202: 381; and Frost et al. (1991) J. Biol. Chem. 266: 18082. A standard curve using known concentrations of biomarker can be used to determine the concentration of biomarker in the sample.

Other ELISA formats familiar to those in the art may also be used, such as using direct adsorption to the plate, rather than a capture antibody, to immobilize the biomarker in the microtiter well. Competitive ELISA may also be used, in which a biomarker in a sample is detected by its ability to compete with labeled biomarker molecules present in the assay solution for binding to the plate. The higher the concentration of biomarker polypeptide in the sample of interest, the more it will block the binding of labeled biomarkers, thus lowering the observed signal.

Lateral flow format immunoassays (immunochromatographic assay) may also be used, in which an aqueous sample is drawn over a surface by capillary action. The surface has a first zone in which is deposited a detection reagent (such as a detectably labeled antibody) and a second zone comprising an immobilized capture reagent (e.g. an antibody). Both the capture reagent and detection reagent specifically bind to the biomarker of interest. As the sample flows across the first zone the detection reagent is solubilized and binds to any analyte (biomarker) present in the sample to form a complex. As the sample continues to flow it contacts the second zone, where any complexes are bound to the capture reagent and concentrated. When a colored particle is used as the detectable label, the concentration of particles at the second zone results in a visible color signal. The level of analyte (biomarker) may then be assessed qualitatively or quantitatively by the intensity of the signal at the second zone.

Biomarker levels may also be determined by Radioimmunoassay (RIA). RIA involves mixing known quantities of radioactive analyte (e.g., labeled with ¹³¹I and ¹²⁵1-tyrosine) with antibody to that analyte, in the presence or absence of unlabeled or “cold” analyte from a sample of interest, and measuring the amount of labeled analyte displaced. In this case the analyte is a biomarker of the present invention. Analyte in the sample will compete with labeled analyte and reduce its binding to the antibody. Unbound analyte is removed, and labeled bound analyte is quantitated. Unbound analyte can be removed, for example, by precipitating the analyte-antibody complexes with a secondary antibody directed against the primary antibody. In another embodiment, the analyte-specific antibodies can be immobilized on the walls of a test tube or microtiter well or to some other solid substrate, so that unbound analyte can be simply washed away.

Any other suitable assay format may be used to detect the biomarker of interest, such as nephelometry/turbidimetry, specifically immunoturbidimetry, which involves measurement of light scattering caused by suspended insoluble antigen (biomarker)/antibody complexes. See, e.g. U.S. Pat. No. 4,605,305. Other methods include radial immunodiffusion (RID), which is observation of a precipitin ring generated by complex formation between an antigen (biomarker) and an antibody, e.g. in an agar/agarose slab. See, e.g. U.S. Pat. No. 3,947,250. Such formats are commonly used in clinical assays.

In other embodiments, the IBD biomarker may be detected by mass spectrometric methods. Mass spectrometric methods include time-of-flight, magnetic sector, quadrupole filter, ion trap, ion cyclotron resonance, electrostatic sector analyzer and hybrids of these. In such embodiments, the IBD biomarker in the sample can be identified and quantified using isotope labeled identical synthetic peptides spiked into the sample. In one embodiment, the mass spectrometer is a laser desorption/ionization mass spectrometer. In laser desorption/ionization mass spectrometry, analytes are placed on the surface of a mass spectrometry probe, which presents an analyte for ionization. A laser desorption mass spectrometer employs laser energy, typically from an ultraviolet or infrared laser, to volatilize and ionize analytes for detection by the ion optic assembly. In another mass spectrometric embodiment, the sample is optionally chromatographically fractionated, and IBD biomarker is then captured on a bio-affinity resin, e.g. a resin derivatized with an antibody. The biomarker is then eluted from the resin and analyzed by MALDI, electrospray, or another ionization method for mass spectrometry. In yet another embodiment, the sample is fractionated on an anion exchange resin and detected directly by MALDI or electrospray mass spectrometry.

In other embodiments, the level of gene expression of biomarker genes may be determined. Gene expression at the nucleic acid level can be quantitated by any method known in the art, including but not limited to, Northern blot analysis, gene chip expression analysis, or RT-PCR (real-time polymerase chain reaction). See e.g., Smith et al. (1993) J. Clin. Endocrin. Metab. 77(5): 1294; Cohen et al. (1993) J. Clin. Endocrin. Metab. 76(4): 1031; Dawczynski et al. (2006) Bone Marrow Transplant. 37:589; and Clemmons et al. (1991) J. Clin. Endocrin. Metab. 73:727.

Northern blot analysis is a standard method for detection and quantitation of mRNA. RNA is isolated from a sample to be assayed (e.g., colonic mucosa). RNA is separated by size by electrophoresis in an agarose gel under denaturing conditions, transferred to a membrane, crosslinked, and hybridized with a labeled probe. In one embodiment of the invention, Northern blot analysis involves radiolabeled or nonisotopically detectably labeled nucleic acids as hybridization probes. In one embodiment of the invention, the membrane holding the RNA sample is prehybridized, or “blocked,” prior to probe hybridization to reduce non-specific background. Unhybridized probe is removed by washing. The stringency of the wash may be adjusted as is well understood in the art. If a radiolabeled (or luminescent) probe is used, the blot can be exposed to film for autoradiography e.g, in the presence of a scintillant. If a nonisotopic probe is used, the blot must typically be treated with nonisotopic detection reagents to develop the detectable probe signal prior to film exposure. The relative levels of expression of the genes being assayed can be quantified using, for example, densitometry or visual estimation. The observed expression level may be normalized to the expression level of an abundantly expressed control gene (e.g. ubiquitin).

In another embodiment, biomarker expression is determined using a gene chip (probe array). A biological sample of interest is prepared and hybridized to the chip, which is subsequently washed, stained and scanned. The data are then processed. Target preparation may entail preparing a biotinylated target RNA from the sample to be tested. The target hybridization step may involve preparing a hybridization cocktail, including the fragmented target, probe array controls, BSA, and herring sperm DNA. In one embodiment, the target is hybridized to the probe array for 16 hours, which probe is washed, stained with streptavidin phycoerythrin conjugate and scanned for light emission at 570 nm. The amount of light emitted at 570 nm is proportional to the target bound at each location on the probe array. Computer analysis using commercially available equipment and software is possible (Affymetrix, Santa Clara, Calif., USA).

In a different embodiment, biomarker expression is determined using real time PCR (RT-PCR). Design of the primers and probes required for RT-PCR of the biomarkers of the present invention is within the skill in the art, in light of the sequences provided herein. In one embodiment, RNA is isolated under RNAse free conditions and converted to DNA using reverse transcriptase, as is well known in the art. RT-PCR probes depend on the 5′-3′ nuclease activity of (e.g., Taq) DNA polymerase to hydrolyze an oligonucleotide hybridized to the target amplicon (biomarker gene). RT-PCR probe oligonucleotides have a fluorescent reporter dye attached to the 5′ end and a quencher moiety coupled to the 3′ end (or vice versa). These probes are designed to hybridize to an internal region of a PCR product. During amplification, the 5′-3′ nuclease activity of the polymerase cleaves the probe, decoupling the fluorescent dye from the quencher moiety. Fluorescence increases in each cycle as more and more probe is cleaved. The resulting fluorescence signal is monitored in real time during the amplification on standard, commercially available equipment. The quantity of biomarker RNA in a sample being evaluated may be determined by comparison with standards containing known quantities of amplifiable RNA.

Biomarkers or biomarker gene expression may be detected using commercially available kits (e.g, as disclosed in the Examples), or using custom assays with commercially available anti-biomarker antibodies obtained from suppliers well known in the art, or using custom assays and antibodies raised by the investigator.

One of skill in the art would recognize that the detection means disclosed herein inherently involve the transformation of an article from one state into another state. Typically the detection means disclosed herein involve transforming an analyte (i.e. the substance to be detected, such as a biomarker polypeptide or an mRNA encoding that polypeptide) into a complex with a detection reagent (e.g. an antibody or complementary nucleic acid). For example, immunological detection means like ELISA, Western blot, etc. involve transformation of biomarker polypeptides into antigen-antibody complexes, which complex formation is essential to the detection. In another example, hybridization-based detection means like amplification (e.g. TaqMan®), Southern/Northern blotting and gene chip-based methods involve transformation of an mRNA encoding the biomarker from a single stranded state to a double stranded state, which complex formation is essential to the detection,

Much of the data reported herein (e.g. in many of the Figures) reflect values obtained from different animals or human subjects. In some embodiments of the present invention the samples to be compared will be obtained from the same subject (e.g. an IBD patient undergoing treatment with an IL-23 antagonist), and thus will be to some degree “internally controlled.” In such embodiments, the ability to discern changes in protein or gene expression levels will be limited only by the inherent precision of the assay, and will not include individual-to-individual variation. Accordingly, small differences between samples from a single subject may be statistically significant even when similar data that include individual-to-individual variation would not be.

### VI. DATA ANALYSIS

Expression levels of the biomarkers of the present invention may be used, depending on the samples being compared, for various purposes, including but not limited to, diagnosing disease, staging patients, monitoring disease status, selecting patients for treatment with an IL-23 antagonist, confirming target engagement, and monitoring therapeutic efficacy. Typically, such methods involve comparing the level(s) of one or more biomarkers of the present invention in sample(s) obtained from a subject of interest (the “subject”) to the level(s) in “reference sample(s)”. For the biomarkers of the present invention, higher levels correlate with disease, or more severe disease status. As used herein, “level(s) of biomarkers in a subject” and similar phrases refer to levels determined in samples obtained from the subject, e.g. serum, blood, urine, feces, etc.

For example, IBD may be diagnosed in a subject by comparing the biomarker level(s) in the subject with level(s) in one or more reference sample(s) comprising individuals that do not suffer from IBD. The diagnosis will typically be based on the judgment of a medical practitioner, and may be based, e.g., on whether the level in the subject is increased by a predetermined multiple or percentage. Diagnosis may also be based on the absolute level(s) in the subject, although such diagnosis will be implicitly based on comparison with level(s) in reference samples (e.g. from the practitioner's general knowledge, or the level(s) prescribed in professional guidelines). The minimum threshold for a “significant” response will necessarily depend on the precision of the detection method in question, in that for a response to be significant it must necessarily exceed the variability in the assay itself. For example, two-dimensional difference gel electrophoresis (2D-DIGE) can detect differences in the levels of proteins down to ±15%. Viswanathan et al. (2006) Nat. Protocols 1:1351.

IBD disease status in a subject may be monitored by comparing biomarker level(s) in the subject with level(s) in reference sample(s) taken from the subject at previous times, and/or from reference sample(s) comprising individuals that do not suffer from IBD. Such comparisons may also be used to assess target engagement, e.g. engagement of IL-23-driven and/or Th17 disease pathways. IBD disease status may vary over time as a natural pattern of flare and remission, or may vary as the result of therapeutic intervention. Measurement of biomarker level(s) at different time points during a course of therapy (e.g. prior to, during, and/or after therapy) may be used to assess the efficacy of the therapeutic regimen, and optionally to guide modification of the regimen to improve outcomes.

The decision of whether to modify therapy will typically be based on the judgment of a medical practitioner, and may be based, e.g., on whether the measured biomarker level(s) in the subject differ from one another value by a predetermined multiple or percentage. Similarly, the judgment of a medical practitioner will typically be involved in determining whether or not an observed change in the level of expression of the biomarker(s) of the present invention reflects a meaningful change in disease status. Such determination will typically be based, at least in part, in the variability of the specific biomarker assay per se, the typical sample-to-sample variation observed for subjects generally, and the typical difference between level(s) in disease and non-disease samples.

In light of the identification of the biomarkers provided herein, it would be within the skill in the art for medical practitioners to determine the levels of the biomarkers (or their level of expression) in a number of human subjects, both with and without IBD. Such data would likely be accumulated in the course of clinical trials assessing the safety and efficacy of a drug (e.g. an IL-23 antagonist antibody) in question. Such biomarker data are often collected in the course of clinical trials, and represent no more than the usual level of effort expended in the art. These baseline data would also be analyzed for variability using standard statistical approaches to determine the precision of the assay(s) in question. Armed with the difference in biomarker level, and the statistical variability in the assay used to measure the biomarker, a skilled medical practitioner would be able to judge whether the level of the biomarker in a given sample was consistent with active IBD (e.g. as a component of diagnosis), and/or the degree to which a given treatment regimen was returning the level of the biomarker toward non-disease level (e.g. in treatment of a patient or conduct of a clinical trial).

Several of the Examples herein (below) provide exemplary analyses for what levels of biomarkers (or their gene expression) in a given sample would be considered consistent with active IBD, based on human data provided in various Figures. Such exemplary analyses may find use not only in diagnosis of IBD, but also in monitoring therapeutic responses to treatment for IBD. Post-treatment values near the value for active IBD would reflect a lack of therapeutic efficacy, whereas values near the control/remission levels would reflect efficaciousness. The rough analysis provided in the Examples would, of course, be updated in light of actual clinical data once available, as is common in the art (and discussed in the preceding paragraph).

The broad scope of this invention is best understood with reference to the following examples, which are not intended to limit the inventions to the specific embodiments.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DIVA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 1, John Wiley and Sons, Inc., New York; Ausubel et al. (2001) Current Protocols in Molecular Biology, Vol, 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protcols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.: Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalog, Molecular Probes, Inc., Eugene, Oreg.; Sigma-Aldrich (2003) Catalog, St. Louis, Mo.

Standard methods of histology of the immune system are described. See, e.g., Muller-Harmelink (ed.) (1986) Human Thymus. Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Statistical analysis may be performed using commercially available software, including but not limited to JMP® Statistical Discovery Software, SAS Institute Inc., Cary, N.C., USA.

Biomarker levels may be determined using commercially available kits or commercially available antibodies, as detailed in the Examples below. Unless otherwise indicated, commercial kits (such as ELISA kits) are used substantially as suggested by the manufacturer. Alternatively, ELISAs and other immunological assays may be developed using commercially available antibodies, or antibodies may be raised to biomarkers for which suitable antibodies are not commercially available. Biomarker gene expression may be monitored using commercially available probes or primers, or such probes or primers may be custom synthesized based on the nucleic acid sequences disclosed herein (either in the sequence listing or disclosed by accession number and incorporated by reference).

### Example 2

### Identification of Potential IBD Biomarkers in Human Samples

Potential biomarkers for IBD were identified by comparison of human serum samples obtained from a control (50-year old woman, non-IBD), a Crohn's disease patient (47-year-old woman with untreated Crohn's disease) and a Crohn's disease patient in remission (39-year-old woman treated with the anti-TNFα antibody infliximab). Samples were obtained from Mayo Clinic (Rochester, Minn., USA).

Proteins<30 kDa were enriched, trypsin digested and purified. Two-dimensional liquid chromatography tandem mass spectroscopy (2D-LC-MS/MS) was then performed as follows. First dimension liquid chromatography involved peptide separation on a strong cation exchange (SCX) column (with 72 fractions collected), and the second dimension comprised a reverse phase column. The resulting fractions were subjected to tandem MS, and the number of appearances of peptides from a given protein in different fractions was scored. The control sample showed 172 proteins, the Crohn's disease sample showed 178 proteins, and the remission sample showed 128 proteins, with 332 total different protein observed overall.

Results showed differential levels of several proteins, including CCL-20/MIP-3a, which was observed in the active Crohn's disease sample but not in control or remission samples. See Example 5 for confirmation of the differential expression of CCL-20/MIP-3a in Crohn's and control serum samples.

### Example 3

### Identification of Potential IBD Biomarkers in Mouse IBD Model

Potential biomarkers for IBD were also obtained by screening for differentially expressed proteins in various samples obtained from IBD mice (plasma, feces, colon lavage, colon tissue lysates and colon epithelial cells) in 2D-DIGE shotgun proteomic experiments, as described generally below.

A mouse model of IBD was generated, as follows. RAG2 KO mice were administered agonist anti-CD40 antibodies (100-200 μg/animal, e.g. 125 μg/animal, i.v.) to generate a mouse model of IBD (“IBD mice”) involving systemic and local inflammatory disease characterized by wasting disease, splenomegaly, increase in serum inflammatory cytokines and colitis. Uhlig et al. (2006) Immunity 25:309 (reporting similar findings with RAG1 KO mice). Unless otherwise indicated, “IBD mouse” as used herein refers to the anti-CD40 RAG KO mouse model described in this example, rather than the T cell transfer model described in Example 18. Naïve RAG KO mice not administered anti-CD40 antibodies served as non-disease controls. Some groups of IBD mice were treated with an anti-mouse-IL-23p19 antibody (mAb 490, Schering-Plough Biopharma), an anti-mouse-IL-23R antibody (mAb 21A4, Schering-Plough Biopharma), or an anti-mouse-IL-12/23p40 antibody, while other IBD mice were treated with an isotype control antibody. All treatment antibodies were administered at 0.5-1 mg/animal i.v. on Day −1 (i.e. the day prior to disease induction). Anti-CD40 was then administered i.p. on Day 0, and unless otherwise indicated animals were monitored for seven days and then sacrificed at Day 7. “Treatment.” as used in this Example, encompasses administration of a therapeutic antibody prior to the disease-inducing agent (in this case, anti-CD40 antibody). Timepoints were generally taken at Days 1, 3, 5 and 7. Plasma, colon lavage, feces and colon tissue samples were collected. As used herein, “IBD mice” refers to RAG KO mice treated with anti-CD40 antibodies, whether “treated” with anti-IL-23p19 or anti-IL-23R antibody or not, and “control” or “non-disease” mice refers to RAG KO mice that were not administered anti-CD40 antibodies.

### Plasma Samples

Mouse plasma samples were screened for potential IBD biomarkers as follows. Low molecular weight proteins (<30 kDa) were analyzed by surface-enhanced laser desorption/ionization time-of-flight (SELDI-TOF) mass spectroscopy. Separate 2D-difference gel electrophoresis (2D-DIGE) gels were run for glycoprotein and non-glycoprotein fractions. Proteins that were differentially expressed in the samples (IBD, IBD-treated, and control) were considered potential IBD biomarkers.

Specifically, EDTA-plasma was obtained from animals and stored at −80° C. until use. For each animal, 150 μl of plasma was diluted in 750 μl of Buffer A (Agilent Technologies, Palo Alto, Calif.) (“Agilent”) supplemented with COMPLETE™ EDTA-free cocktail of protease inhibitors (Roche Diagnostics Corp., Indianapolis, Ind.) (“Roche”), filtered on a 0.22 μm spin filter (Agilent), and depleted of high abundant proteins using a 4.6×100 mm Multiple Affinity Removal System for mouse samples (Agilent), following the manufacturer's instructions. This column uses antibodies to remove albumin, IgG, and transferrin from mouse body fluids. Immunoaffinity chromatography was conducted on an AKTA Explorer (GE Healthcare, Piscataway, N.J.) (“GE Healthcare”).

Flow-through fractions were PBS-buffer exchanged and concentrated using a Centricon Plus-20 (Millipore, Bedford, Mass.) centrifugal filter device. Glyco- and non-glycoproteins were then separated using Wheat Germ Agglutinin (WGA) lectin column (Pierce Biotechnology, Rockford, Ill.) (“Pierce”) following the manufacturer's instructions. Samples were then desalted using a Zeba Spin-Column Device (Pierce), and acetone precipitated overnight at −20° C. Proteins pellets were resuspended in 200 μl of lysis buffer comprising 7M urea, 2M thiourea, 1% (3-[(3-cholamidopropyl)dimethylamino]-1-propane sulfonate (CHAPS), 1% TritonX-100, 1% N-decyl-N-N′-dimethyl-3-ammonia-1-propane sulfonate (SB3-10), 1% amidosulfobetaine-14 (ASB-14), 40 mM Tris pH 8.8, 5 mM magnesium acetate, and EDTA-free protease inhibitor cocktail (Roche). Protein concentration was assessed using Coomassie Blue Plus (Pierce).

Glyco- and non-glyco-protein samples were labeled with 200 pmol of CyDye (GE Healthcare) for 50 μg of protein, using Cy 5 or Cy 3 for the samples and Cy 2 for the pooled internal standard, which internal standard comprised a mixture of an equal weight amount of each sample. See Alban et al. (2003) Proteomics 3:36. Labeling was quenched with 5 μl of 10 mM lysine after a 30 min incubation on ice in the dark. One volume of 2× sample buffer was added to each sample, which sample buffer comprised 6M urea, 2M thiourea, 1% CHAPS, 1% TritonX-100, 1% SB3-10, 1% ASB 14, 65 mM DTT, 4% Pharmalytes® 3-10 (GE Healthcare). The Cy 2, Cy 3 and Cy 5 labeling reactions were then combined, and rehydration buffer (6M urea, 2M thiourea, 1% CHAPS, 1% TritonX-100, 1% SB3-10, 1% ASB 14, 6.5 mM DTT, 1% Pharmalytes® 3-10 (GE Healthcare)) was added up to 450 μl per gel.

The first dimension separation of the 2D-DIGE was carried out with an IPGphor isoelectric focusing unit (GE Healthcare) by applying the samples to 24 cm strips, pH 4 to pH 7, for 12 h for rehydration, followed by isoelectric focusing for a total of 90 kV-hours. Strips were then equilibrated in 6 M urea, 2 M thiourea, 2% SDS, 0.1 M Tris pH 6.8 with 0.5% DTT (w/v) for 15 min, and then with 4.5% (w/v) iodoacetamide for 15 min. The second dimension of the 2D-DIGE was performed on 12% polyacrylamide gels with the Ettan DALTtwelve system (GE Healthcare) at 4.3 W/gel for 11 hours in the dark. Immediately following 2D-DIGE, gels were scanned using a Typhoon 9400 imager (GE Healthcare). Image analysis was performed using DeCyder™ Biological Variation Analysis (BVA) software v. 6.5.14 (GE Healthcare).

Picking gels were prepared and run in parallel with analytic gels described supra, and loaded with 300 μg of unlabeled glycoprotein and 500 μg of unlabeled non-glycoprotein. Picking gels were fixed overnight in 10% methanol, 7% acetic acid, stained with SYPRO® Ruby (Invitrogen, Carlsbad, Calif.) (“Invitrogen”), and destained overnight. Spots statistically up- or down-regulated in the disease group and coming back to control levels in the treated group were picked, digested and analyzed by mass spectroscopy.

Proteins were detected by mass spectroscopy and identified as described below. Results of screening of mouse plasma revealed that human haptoglobin and orosomucoid 1, among other proteins, may find use as biomarkers for IBD.

### Colon Samples

Samples obtained from mouse colon were also screened for potential IBD biomarkers. Colon samples included whole colon tissue lysates, epithelial cell preparations, feces and lavage.

Mouse feces and colon lavage samples were screened for potential IBD biomarkers by 2D-DIGE analysis of plasma glyco- and non-glycoproteins, as follows. Mouse colon was removed and feces were extracted. Feces were vortexed with 1 ml of PBS supplemented with a COMPLETE™ EDTA free cocktail of protease inhibitors (Roche), and left on ice for 20 minutes. Colon lavage consisted in an injection of cold 1 ml PBS plus protease cocktail of inhibitors into the colon, followed by a 20 minute incubation under gentle agitation at 4° C. Feces and lavages were then centrifuged at 4000 rpm in an Eppendorf micro centrifuge Model 5415-C (Eppendorf AG, Hamburg, Germany) for 5 minutes, and supernatants were precipitated overnight in four volumes of cold acetone at −20° C. Samples were then centrifuged for 25 minutes at 2000 rpm in a Beckman GS-6R centrifuge (Beckman Coulter Inc., Fullerton, Calif.), and proteins pellets were resuspended in 40 to 80 μl of 25 mM Tris pH 8.8/0.4% SDS. An equal volume of each sample was separated by SDS-PAGE on a 4-12% NuPage (Invitrogen, Carlsbad, Calif.) in MES buffer. The gel was stained with SYPRO® Ruby (Invitrogen) overnight and scanned on a Typhoon 9400 imager (GE Healthcare). Relative sample concentrations for use in determining the loading of the preparative gels were obtained using ImageQuant® v5.2 image quantitation analysis software (GE Healthcare) by normalizing the sum of pixels above background in each lane to the sum of pixels above background in the lane with the weakest signal.

A preparative 4-12% NuPage gel was run with an equal weight amount of each sample and stained with GelCode® Blue (Pierce), and bands were manually excised. Proteins were detected by mass spectroscopy and identified as described below. Results of screening of mouse feces and colon lavage revealed that human DMBT, PAP/REG3α, REG3γ, and calprotectin (S100A8/S100A9), among other proteins, may find use as biomarkers for IBD.

### Mass Spectroscopic Detection and Identification of Proteins

For mass spectrometry analysis, excised bands were digested with sequencing-grade modified trypsin on a Progest protein digestion station (Genomic Solutions, Ann Arbor, Mich., USA). Mass spectrometry analysis was done on a LCQ Deca Ion Trap mass spectrometer (Thermo Fisher Scientific Inc., Waltham, Mass., USA) with sample introduction with a 48 well Paradigm ASI autosampler (Michrom Bioresources, Inc., Auburn, Calif., USA) (“Michrom Bioresources”) and a Paradigm MS4 HPLC system (Michrom Bioresources). The column was self-packed with Vydac® C18 resin (5 micron beads, 300 Å pores), 10 cm long with a 15 micron tip (New Objective Inc., Woburn, Mass., USA). The chromatographic separation was done using a linear gradient elution: 8-60% B solvent for 30 min (solvent A: 2% acetonitrile, 0.1% formic acid and 0.005% heptafluorobutyric acid; solvent B: 90% acetonitrile, 0.1% formic acid and 0.005% heptafluorobutyric acid).

LC-MS/MS raw files were searched using the Mascot v2.1.6 (Matrix Sciences Ltd., London, UK) software package against the mouse subset of the National Center for Biotechnology Information (NCBI) non-redundant protein database (updated as of Feb. 28, 2006). See Perkins et al. (1999) Electrophoresis 20:3551. Search parameters included no restriction on molecular weight and pI, fixed modification on cysteine residues (carbamidomethylation), variable modification on methionine residues (oxidation), a peptide mass tolerance of +/−1.5 Daltons, a fragment mass tolerance of +/−0.8 Daltons, and one missed tryptic cleavage. Protein identification was based on at least two matching peptides. Protein hits with only one matching peptide were reviewed manually and included as identifications when a stretch of at least 4 b or y ions were present.

### Mouse Whole Colon and Epithelial Cell Lysates

Whole colon tissue lysates and epithelial cell preparations were subjected to 2D-difference gel electrophoresis (2D-DIGE). Proteins that were differentially expressed in the samples (IBD, IBD-treated, and control), i.e. “IBD-associated proteins,” are considered potential IBD biomarkers, and also potential therapeutic targets. In one study, protein expression was compared by 2D-DIGE between whole colon tissue lysates from control (non-disease) mice, IBD mice and IBD mice treated with anti-mouse-IL-23R antibodies. In these and other experiments, IBD animals exhibited phenotypic characteristics typical of IBD, whereas treated mice (either anti-IL-23R or anti-IL-23p19) showed reduction in disease phenotype, and were in fact phenotypically indistinguishable from control non-disease animals.

Comparison of untreated control and IBD mice revealed proteins that are modulated in IBD, and further comparison with treated samples revealed which of these proteins were reverted by treatment. Nearly 30 proteins modulated in IBD were found to be reverted by treatment. Proteins that were differentially expressed were first identified by an ANOVA p<0.05, and then confirmed as having p<0.05 in a pairwise t-test. Table 3A lists these proteins, as well as their presumed function based on the literature, and further identifying information regarding the presumed human orthologs.

TABLE 3A
IBD-Associated Proteins in Mouse Whole Colon Lysate

| | Presumed | Human | Gene | Human Nucleic Acid/ |
|---|---|---|---|---|
| Mouse Gene | Function | Gene | ID | Protein Sequence |
| Aldehyde | enzyme | ALDH1A2 | 8854 | NM_003888.2 |
| dehydrogenase 1 | | | | NP_003879.2 |
| family, member A2 | | | | |
| Aldehyde | enzyme | ALDH1B1 | 219 | NM_000692.3 |
| dehydrogenase 1 | | | | NP_000683.3 |
| family, member B1 | | | | |
| Dihydrolipoamide | enzyme | DLST | 1743 | NM_001933.3 |
| S- | | | | NP_001924.2 |
| succinyltransferase | | | | |
| (E2 component of | | | | |
| 2-oxo-glutarate | | | | |
| complex) | | | | |
| Enoyl coenzyme A | enzyme | ECH1 | 1891 | NM_001398.2 |
| hydratase 1, | | | | NP_001389.2 |
| peroxisomal | | | | |
| Malate | enzyme | MDH1 | 4190 | NM_005917.2 |
| dehydrogenase 1, | | | | NP_005908.1 |
| NAD (soluble) | | | | |
| NADH | enzyme | NDUFS1 | 4719 | NM_005006.5 |
| dehydrogenase | | | | NP_004997.4 |
| (ubiquinone) Fe—S | | | | |
| Protein 1 | | | | |
| NADH | enzyme | NDUFS2 | 4720 | NM_004550.4 |
| dehydrogenase | | | | NP_004541.1 |
| (ubiquinone) Fe—S | | | | |
| Protein 2 | | | | |
| Peroxiredoxin 6 | enzyme | PRDX6 | 9588 | NM_004905.2 |
| | | | | NP_004896.1 |
| Sulfotransferase | enzyme | SULT1A1 | 6817 | NM_001055.2 |
| family, cytosolic, | | | | NP_001046.2 |
| 1A, phenol- | | | | |
| preferring, member 1 | | | | |
| vinculin | enzyme | VCL | 7414 | NM_014000.2 |
| | | | | NP_054706.1 |
| Actin, gamma 2, | other | ACTG2 | 72 | NM_001615.3 |
| smooth muscle, | | | | NP_001606.1 |
| enteric | | | | |
| Collagen, type VI, | other | COL6A1 | 1291 | NM_001848.2 |
| alpha 1 | | | | NP_001839.2 |
| Desmin | other | DES | 1674 | NM_001927.3 |
| | | | | NP_001918.3 |
| Fibrinogen beta | other | FGB | 2244 | NM_005141.3 |
| chain | | | | NP_005132.2 |
| Gelsolin | other | GSN | 2934 | NM_000177.4 |
| (amyloidosis, | | | | NP_000168.1 |
| Finnish type) | | | | |
| Golgi | other | GOLPH3L | 55204 | NM_018178.4 |
| phosphoprotein 3- | | | | NP_060648.2 |
| like | | | | |
| Heat shock protein | other | HSP90AA1 | 3320 | NM_001017963.2 |
| 90 kDa alpha | | | | NP_001017963.2 |
| (cytosolic), class | | | | |
| A, member 1 | | | | |
| Selenium binding | other | SELENBP1 | 8991 | NM_003944.2 |
| protein 1 | | | | NP_003935.2 |
| Serpin peptidase | other | SERPINB6 | 5269 | NM_004568.4 |
| inhibitor, clade B | | | | NP_004559.4 |
| (ovalbumin), | | | | |
| member 6 | | | | |
| Hsp70 | other | HSPA1A | 3303 | NM_005345.5 |
| | | | | NP_005336.3 |
| Cytidylate kinase | other | CMPK1 | 51727 | NM_001136140.1 |
| | | | | NP_001129612.1 |
| Creatine kinase, | kinase | CKB | 1152 | NM_001823.3 |
| brain | | | | NP_001814.2 |
| Keratin 8 | kinase | KRT8 | 3856 | NM_002273.3 |
| | | | | NP_002264.1 |
| Macrophage | transmembrane | MSR1 | 4481 | NM_138715.2 |
| scavenger receptor | receptor | | | NP_619729.1 |
| 1 | | | | |
| Ribosomal protein | transmembrane | RPSA | 3921 | NM_002295.4 |
| SA | receptor | | | NP_002286.2 |
| ATP synthase, H+ | transporter | ATP5H | 10476 | NM_006356.2 |
| transporting, | | | | NP_006347.1 |
| mitochondrial F0 | | | | |
| complex, subunit d | | | | |
| ATP synthase, H+ | transporter | ATP5B | 506 | NM_001686.3 |
| transporting, | | | | NP_001677.2 |
| mitochondrial F1 | | | | |
| complex, beta | | | | |
| polypeptide | | | | |
| Tu translation | translation | TUFM | 7284 | NM_003321.4 |
| elongation factor, | regulator | | | NP_003312.3 |
| mitochondrial | | | | |
| Chloride | ion channel | CLIC1 | 1192 | NM_001288.4 |
| intracellular | | | | NP_001279.2 |
| channel 1 | | | | |

Table 3B provides the ratios of expression for the in IBD (disease) mice versus naïve mice, and in anti-IL-23R-treated IBD mice versus untreated (disease) IBD mice, as measured at Day 7. Image analysis was performed using DeCyder™ Biological Variation Analysis (BVA) software v. 6.5.14 (GE Healthcare). Ratios are expressed as negative numbers when the denominator is greater than the numerator, i.e. when the ratio would otherwise be less than 1.0. This is done to present all ratios as >1.0 for ease of comparison of the magnitude of the ratios, regardless of the direction of change.

TABLE 3B
Differential Expression of IBD-Associated
Proteins in Mouse Whole Colon Lysate

| | Human | Disease/ | anti-IL-23R/ |
|---|---|---|---|
| Mouse Gene | Gene | Naïve | Disease |
| Aldehyde | ALDH1A2 | −2.25 | 1.98 |
| dehydrogenase 1 | | | |
| family, member A2 | | | |
| Aldehyde | ALDH1B1 | −1.65 | 1.96 |
| dehydrogenase 1 | | | |
| family, member B1 | | | |
| Dihydrolipoamide | DLST | −1.75 | 2.08 |
| S- | | | |
| succinyltransferase | | | |
| (E2 component of | | | |
| 2-oxo-glutarate | | | |
| complex) | | | |
| Enoyl coenzyme A | ECH1 | −1.82 | 1.91 |
| hydratase 1, | | | |
| peroxisomal | | | |
| Malate | MDH1 | −1.71 | 1.57 |
| dehydrogenase 1, | | | |
| NAD (soluble) | | | |
| NADH dehydrogenase | NDUFS1 | −2.12 | 1.85 |
| (ubiquinone) Fe—S | | | |
| Protein 1 | | | |
| NADH dehydrogenase | NDUFS2 | −1.55 | 1.37 |
| (ubiquinone) Fe—S | | | |
| Protein 2 | | | |
| Peroxiredoxin 6 | PRDX6 | −1.54 | 1.36 |
| Sulfotransferase | SULT1A1 | −5.68 | 4.30 |
| family, cytosolic, | | | |
| 1A, phenol- | | | |
| preferring, member | | | |
| 1 | | | |
| vinculin | VCL | −2.06 | 1.84 |
| Actin, gamma 2, | ACTG2 | −2.05 | 1.87 |
| smooth muscle, enteric | | | |
| Collagen, type VI, | COL6A1 | −1.60 | 1.60 |
| alpha 1 | | | |
| Desmin | DES | −2.52 | 2.15 |
| Fibrinogen beta | FGB | 1.98 | −2.05 |
| chain | | | |
| Gelsolin | GSN | −2.31 | 1.90 |
| (amyloidosis, | | | |
| Finnish type) | | | |
| Golgi phosphoprotein | GOLPH3L | −1.94 | 1.80 |
| 3-like | | | |
| Heat shock protein | HSP90AA1 | 4.42 | −3.05 |
| 90 kDa alpha | | | |
| (cytosolic), class | | | |
| A, member 1 | | | |
| Selenium binding | SELENBP1 | −3.90 | 4.05 |
| protein 1 | | | |
| Serpin peptidase | SERPINB6 | −1.58 | 1.37 |
| inhibitor, clade B | | | |
| (ovalbumin), | | | |
| member 6 | | | |
| Hsp70 | HSPA1A | −1.98 | 1.78 |
| Cytidylate kinase | CMPK1 | −1.66 | 1.45 |
| Creatine kinase, | CKB | −2.35 | 1.89 |
| brain | | | |
| Keratin 8 | KRT8 | −2.46 | 2.15 |
| Macrophage | MSR1 | −1.67 | 1.53 |
| scavenger receptor 1 | | | |
| Ribosomal protein | RPSA | 1.39 | −1.41 |
| SA | | | |
| ATP synthase, H+ | ATP5H | −1.34 | 1.38 |
| transporting, | | | |
| mitochondrial F0 | | | |
| complex, subunit d | | | |
| ATP synthase, H+ | ATP5B | −1.60 | 1.82 |
| transporting, | | | |
| mitochondrial F1 | | | |
| complex, beta | | | |
| polypeptide | | | |
| Tu translation | TUFM | −1.31 | 1.27 |
| elongation factor, | | | |
| mitochondrial | | | |
| Chloride | CLIC1 | 1.39 | −1.46 |
| intracellular | | | |
| channel 1 | | | |

In another study, protein expression as measured by 2D-DIGE was compared between colon epithelial cells from control (non-disease) mice, IBD mice and IBD mice treated with an anti-mouse-IL-23R antibody. Comparison of untreated control and IBD mice revealed proteins that are modulated in IBD, and further comparison with treated samples revealed which of these proteins were reverted by treatment. Seven proteins modulated in IBD were found to be reverted by treatment. Table 4A lists these proteins, as well as their presumed function based on the literature, and further identifying information regarding the presumed human orthologs.

TABLE 4A
IBD-Associated Proteins in Mouse Colonic Epithelial Cells

| | | | | Human Nucleic Acid/ |
|---|---|---|---|---|
| | Presumed | | | Protein Sequence |
| Mouse Gene | Function | Human Gene | GeneID | Accession Numbers |
| Carbonic | enzyme | CA1 | 759 | NM_001128829.1 |
| Anhydrase I | | | | NP_001122301.1 |
| Cytochrome c-1 | enzyme | CYC1 | 1537 | NM_001916.3 |
| | | | | NP_001907.2 |
| Enoyl coenzyme A | enzyme | ECH1 | 1891 | NM_001398.2 |
| hydratase 1, | | | | NP_001389.2 |
| peroxisomal* | | | | |
| Peroxiredoxin 6* | enzyme | PRDX6 | 9588 | NM_004905.2 |
| | | | | NP_004896.1 |
| Sulfotransferase | enzyme | SULT1B1 | 27284 | NM_014465.3 |
| family, cytosolic, | | | | NP_055280.2 |
| 1B, member 1 | | | | |
| Apolipoprotein A-I | transporter | APOA1 | 335 | NM_000039.1 |
| | | | | NP_000030.1 |
| Selenium binding | other | SELENBP1 | 8991 | NM_003944.2 |
| protein 1* | | | | NP_003935.2 |

*also listed in Table 3A

Table 4B provides the ratios of expression for the in IBD (disease) mice versus naïve mice, and in anti-IL-23R-treated IBD mice versus untreated (disease) IBD mice, as measured at Day 7. Image analysis was performed using DeCyder™ Biological Variation Analysis (BVA) software v. 6.5.14 (GE Healthcare). Ratios are expressed as negative numbers when the denominator is greater than the numerator, i.e. when the ratio would otherwise be less than 1.0. This is done to present all ratios as >1.0 for ease of comparison of the magnitude of the ratios, regardless of the direction of change.

TABLE 4B
Differential Expression of IBD-Associated
Proteins in Mouse Colonic Epithelial Cells

| | | Disease/ | anti-IL-23R/ |
|---|---|---|---|
| Mouse Gene | Human Gene | Naïve | Disease |
| Carbonic | CA1 | −3.19 | 3.17 |
| Anhydrase I | | | |
| Cytochrome c-1 | CYC1 | −1.27 | 1.46 |
| Enoyl coenzyme | ECH1 | −1.44 | 1.48 |
| A hydratase 1, | | | |
| peroxisomal | | | |
| Peroxiredoxin 6 | PRDX6 | −5.94 | 2.05 |
| Sulfotransferase | SULT1B1 | −1.76 | 1.54 |
| family, cytosolic, | | | |
| 1B, member 1 | | | |
| Apolipoprotein A-I | APOA1 | −2.74 | 2.79 |
| Selenium binding | SELENBP1 | −2.65 | 1.91 |
| protein 1 | | | |

All sequence information and other information contained within the database entries referenced in Tables 3A and 4A is hereby expressly incorporated by reference in its entirety. GenBank accession numbers are provided only for the predominant or longest isoforms in cases where multiple isoforms are known. Accession numbers for other isoforms are disclosed in the GeneID records as of Sep. 23, 2008. These results suggest that human orthologues of these mouse genes may be useful as biomarkers of IBD, particularly when detected in tissue samples, e.g. from colon biopsies.

It is also possible that one or more of the proteins listed in Tables 3A or 4A is functionally involved in the pathogenesis of IBD, and as such could serve as a target for pharmaceutical intervention or as a therapeutic agent itself. In therapy, antagonists would be used for IBD-associated proteins that are overexpressed in disease, and agonists would be used for IBD-associated proteins that are underexpressed in disease. Such a target protein could be used in drug discovery, for example in an assay to screen for small molecule drugs or as an antigen (e.g. an immunogen) for raising an antibody. Any resulting anti-target antibodies raised in a non-human animal could then be modified. e.g. by chimerization or humanization, for therapeutic use in humans. The genes encoding such protein targets could also be used to design nucleic-acid-based antagonists, such as siRNA or antisense nucleic acids. For situations calling for use of agonists of the target, such agonists would include the target protein itself (or a biologically active fragment or variant thereof), a small molecule and an agonist antibody.

### Mouse Feces and Lavage

Feces and lavage samples from colon were run on 1D-gels to resolve proteins ranging from 6 kDa to 200 kDa. DMBT1 (Deleted in Malignant Brain Tumors 1), MIF (Macrophage migration Inhibitory Factor), LCN2 (Lipocalin 2), REG3β and REG3γ (Regenerating islet-derived 3 alpha and gamma) and S100A8 and S100A9 (collectively calprotectin) were all found to be upregulated in disease and reverted by both anti-IL-23R and anti-IL-23p19 treatment. Once these potential biomarkers were identified by differential expression in feces and lavage samples, they were further validated by measurement of gene and protein expression in colon tissue, by TaqMan® real-time quantitative PCR analysis and Western blot, respectively. Unless otherwise indicated samples were obtained 7 days after treatment.

### Example 4

### Validation of IBD Biomarkers

Potential biomarkers identified in the previous two examples were further validated by analysis of samples obtained IBD mice and human samples, as follows, and as discussed in greater detail for specific biomarkers in subsequent Examples.

Selected potential IBD biomarkers were further validated using TaqMan® real time quantitative PCR analysis and Western blots in a time-course study in IBD mice, in which the differential expression was monitored to confirm that it followed the time course of the disease state in the model. Selected potential mouse model IBD biomarkers were also validated using dose response studies, in which differential expression was monitored as a function of the dose of IL-23 antagonist. Primers were generated against a selection of the human orthologs of the mouse genes identified in the shotgun proteomic analysis. Messenger RNA was prepared from colon tissue from control, disease, anti-p19 and anti-IL-23R treated mice, and real-time PCR was carried out, as described for various biomarkers in the following examples.

Human tissues were used to confirm that the human orthologs of some of the IBD biomarkers discovered by shotgun proteomic analysis in the mouse IBD model are useful biomarkers of human IBD. Some of the biomarkers of the present invention were validated using human colon biopsy samples from control subjects, active Crohn's disease patients and Crohn's disease patients in remission obtained from the Mayo Clinic (Rochester, Minn., USA). Some of the biomarkers of the present invention were validated using human serum samples from active Crohn's disease patients, UC patients, or sex and age-matched normal controls for each, obtained from Bioreclamation, Inc. (Liverpool, N.Y., USA). Bioreclamation samples were sex and age-matched (±5 years).

Potential biomarkers that were found to correlate with IBD in both mouse and human experiments were selected as likely to be valuable biomarkers of IBD, and particularly in the monitoring of IBD in human subjects being treated with anti-IL-23p19 or anti-IL-23R antibodies.

### Example 5

### CCL20

CCL20 (chemokine ligand 20, MIP-3α) was found at elevated levels in both Crohn's disease and ulcerative colitis serum samples as compared to control samples. FIG. 1. CCL20 was detected using the Quantikine® human CCL20-MIP-3α Immunoassay kit (catalog DM3A00) from R&D Systems® (Minneapolis, Minn., USA).

The results indicate that there is at least about a 3-fold increase in serum CCL20 levels in Crohn's disease and ulcerative colitis patients compared with controls. Accordingly, an assay indicating a 3-fold increase in serum CCL20 over normal levels would be consistent with the presence of Crohn's disease, whereas a 30% increase over normal levels would not seem particularly significant (even if statistically reliable). In this and other Examples, the recited fold-increase in disease biomarker levels is based on differences between the bulk of data points, rather than precisely the average or median, so as to minimize the effects of outlier points that might otherwise skew the analysis.

### Example 6

### DMBT1

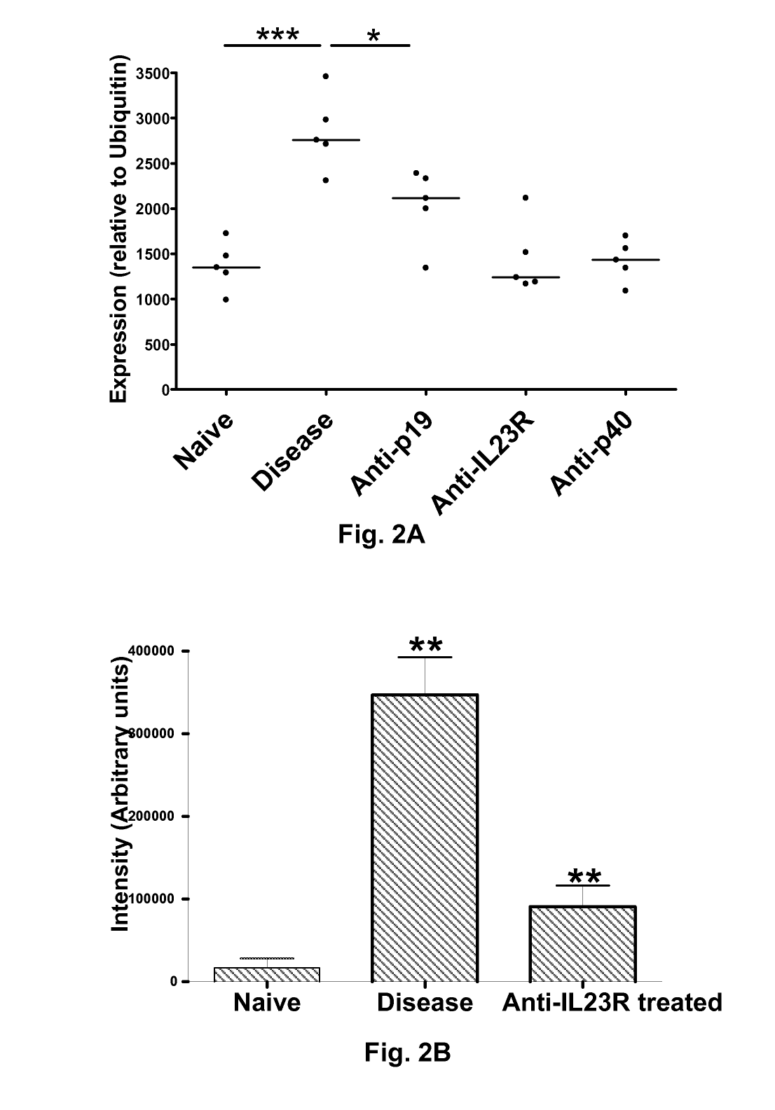

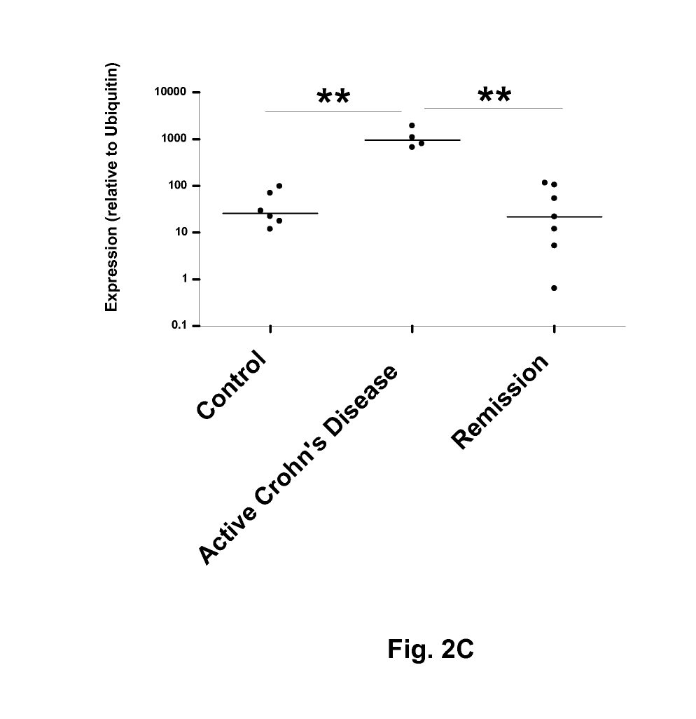

DMBT1 gene expression was two-fold higher in IBD mice compared with control mice, and treatment with anti-IL-23R or anti-IL-12/23p40 antibodies (and to a lesser extent anti-IL-23p19 antibody) reverted DMBT1 to nearly control levels. FIG. 2A. DMBT1 protein expression was highly upregulated in disease, and significantly (although incompletely) reverted by treatment with anti-IL-23R. FIG. 2B. Further experiments (data not shown) demonstrated that treatment with anti-IL-23R antibody maintained. DMBT1 protein expression in colon epithelial cells at pre-disease levels at Days 1, 3, 5 and 7, whereas untreated IBD mice showed elevated DMBT1 expression at Day 3, with maximal expression at Days 5 and 7. Reversion of DMBT1 gene expression in mouse colon was generally dose responsive, with higher doses of anti-IL-23p19 antibody or anti-IL-23R antibody showing more complete reversion than lower doses (data not shown).

DMBT1 was further validated by gene expression analysis of human gut biopsies from active Crohn's patients, Crohn's disease patients in remission, and control subjects. FIG. 2C. DMBT1 gene expression was upregulated in the active Crohn's samples but reverted to nearly non-disease levels in the remission samples.

The results (FIG. 2C) indicate that there is more than a log (˜30-fold) increase in DMBT1 expression in Crohn's patients (gut biopsy) compared with Controls or patients in remission. Accordingly, an assay indicating a 20-fold increase in DMBT1 over normal levels in a gut biopsy would be consistent with the presence of Crohn's disease, whereas a two-fold increase over normal levels would not seem particularly significant (even if statistically reliable).

### Example 7

### MIF

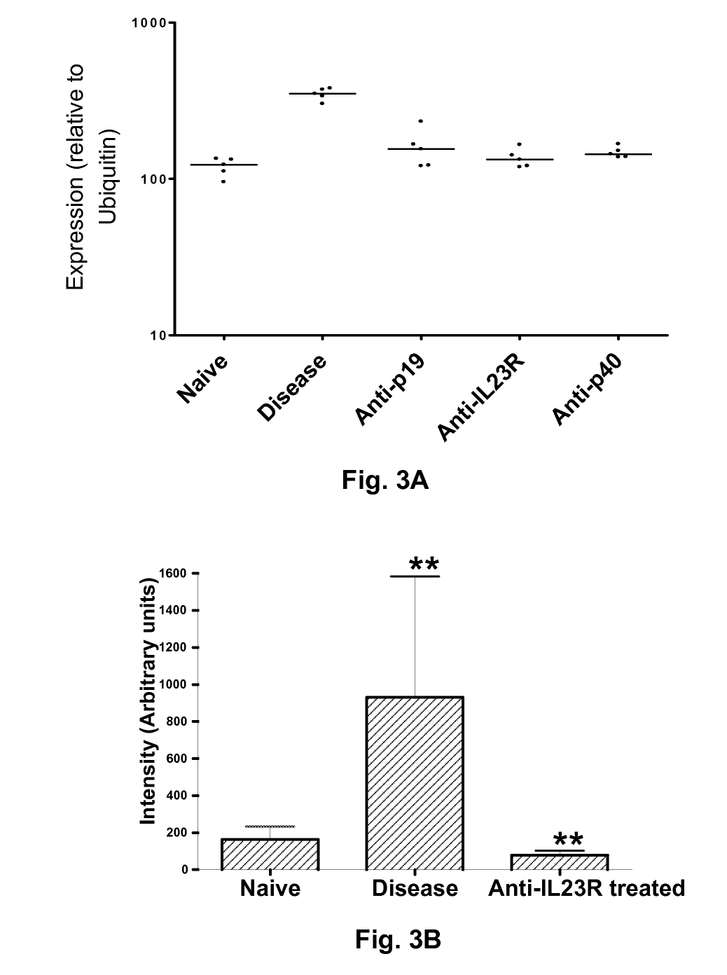

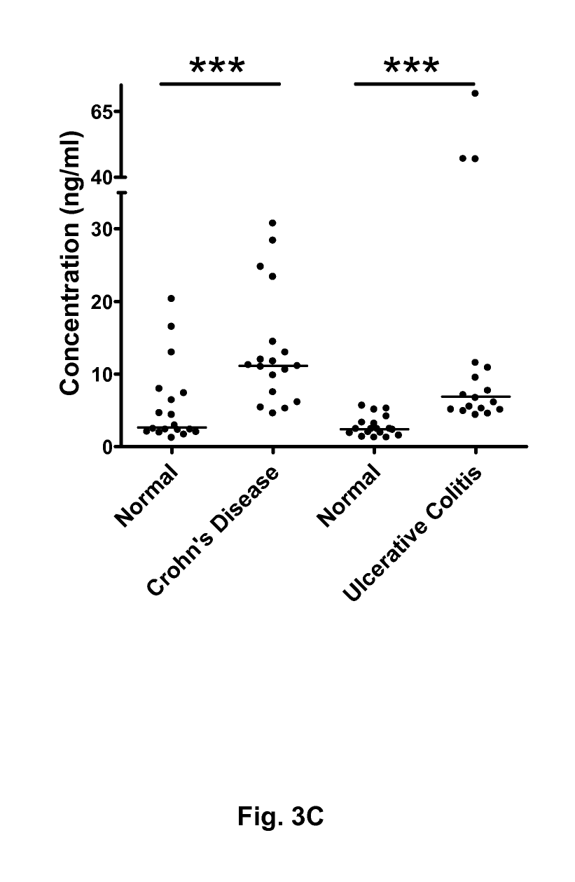

MIF gene expression was elevated in IBD mice compared with control mice, and treatment with anti-IL-23R or anti-IL-12/23p40 antibodies (and to a lesser extent anti-IL-23p19 antibody) reverted MIF to nearly control levels. FIG. 3A. MIF protein levels were elevated in disease, and reverted by treatment with anti-IL-23R. FIG. 3B. Further experiments (data not shown) demonstrated that treatment with anti-IL-23R antibody reduced MIF gene expression to nearly pre-disease levels by Days 5 and 7, although it was less effective at Days 1 and 3. Untreated IBD mice showed maximal MIF gene expression from Day 1 (the earliest point tested) onward. Reversion of MIF gene expression was generally dose responsive, with higher doses of anti-IL-23p19 antibody or anti-IL-23R antibody showing more complete reversion than lower doses (data not shown).

MIF was further validated by ELISA of human serum samples from Crohn's disease and ulcerative colitis patients and control subjects. FIG. 3C. Human MIF was detected using the Quantikine® human MIF Immunoassay kit (catalog DMF00) from R&D Systems® (Minneapolis, Minn., USA). MIF expression was elevated in both the Crohn's disease and ulcerative colitis samples compared with controls. Published data show that elevated MIF levels in the plasma of Crohn's patients are at least partially reverted toward control levels by treatment with the anti-TNFα antibody infliximab. De Jong et al. (2001) Nature Immunol. 2:1061 (FIG. 1 therein).

The results (FIG. 3C) indicate that there is at least about a 5-fold increase in serum MIF levels in Crohn's disease patients compared with controls, and about a 2-fold increase in serum MIF levels in ulcerative colitis patients. Accordingly, an assay indicating a 3-fold increase in serum MIF over normal levels would be consistent with the presence of Crohn's disease, whereas a 50% increase over normal levels would not seem particularly significant (even if statistically reliable). An assay indicating a 2-fold increase in serum MIF over normal levels would be consistent with the presence of ulcerative colitis (or Crohn's disease of course), although such an increase may not be statistically significant given the scatter in the values.

In addition, FIGS. 11G and 12G show that MIF is elevated in the mouse T cell transfer colitis model described at Example 18, and that treatment with anti-IL-23p19 antibody reduces the level as compared to animals treated with an isotype control antibody under both the prophylactic (FIG. 11G) and treatment (FIG. 12G) protocols.

### Example 8

### LCN2

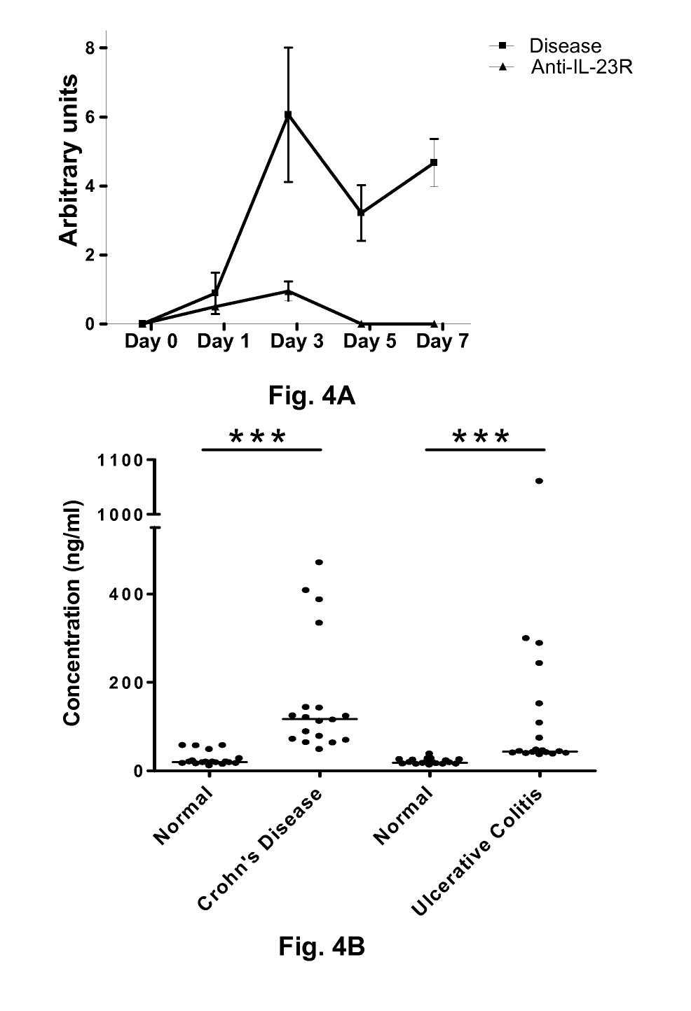

A time course study demonstrated that LCN2 was elevated in lamina propria of IBD mice after administration of antiCD40 antibody. Although LCN2 was undetectable in control mice, it was detectable at Day 1 and reached maximum level at Day 3 onward in IBD mice. FIG. 4A. Treatment with anti-IL-23R antibody significantly reduced LCN2 levels at Day 3, and reverted to control (undetectable) levels at Days 5 and 7. Reversion of LCN2 gene expression was generally dose responsive, with higher doses of anti-IL-23p19 antibody or anti-IL-23R antibody showing more complete reversion than lower doses (data not shown).

LCN2 was further validated by ELISA of human serum samples from Crohn's disease and ulcerative colitis patients and control subjects. FIG. 4B. Human LCN2 was detected using the human Lipocalin-2/NGAL DuoSet®ELISA Development System (catalog DY1757) from R&D Systems® (Minneapolis, Minn., USA). LCN2 expression was elevated in the Crohn's disease and ulcerative colitis samples compared with control.

The results (FIG. 4B) indicate that there is about a 5-fold increase in serum LCN2 levels in Crohn's patients compared with Controls. Accordingly, an assay indicating a 3-fold increase in serum LCN2 over normal levels would be consistent with the presence of Crohn's disease, whereas a 50% increase over normal levels would not seem particularly significant (even if statistically reliable).

LCN2 may also serve as a useful marker to distinguish Crohn's disease from ulcerative colitis. FIG. 4B demonstrates that all Crohn's patients exhibit levels of calprotectin greater than about 50 ng/ml, whereas only 7 of 18 ulcerative colitis subjects do. See Example 29.

In addition, FIGS. 11F and 12F show that show that LCN2 is elevated in the mouse T cell transfer colitis model described at Example 18, and that treatment with anti-IL-23p19 antibody dramatically reduces the level as compared to animals treated with an isotype control antibody under both the prophylactic (FIG. 11F) and treatment (FIG. 12F) protocols.

### Example 9

### Murine REG3β and REG3γ and human PAP/REG3α

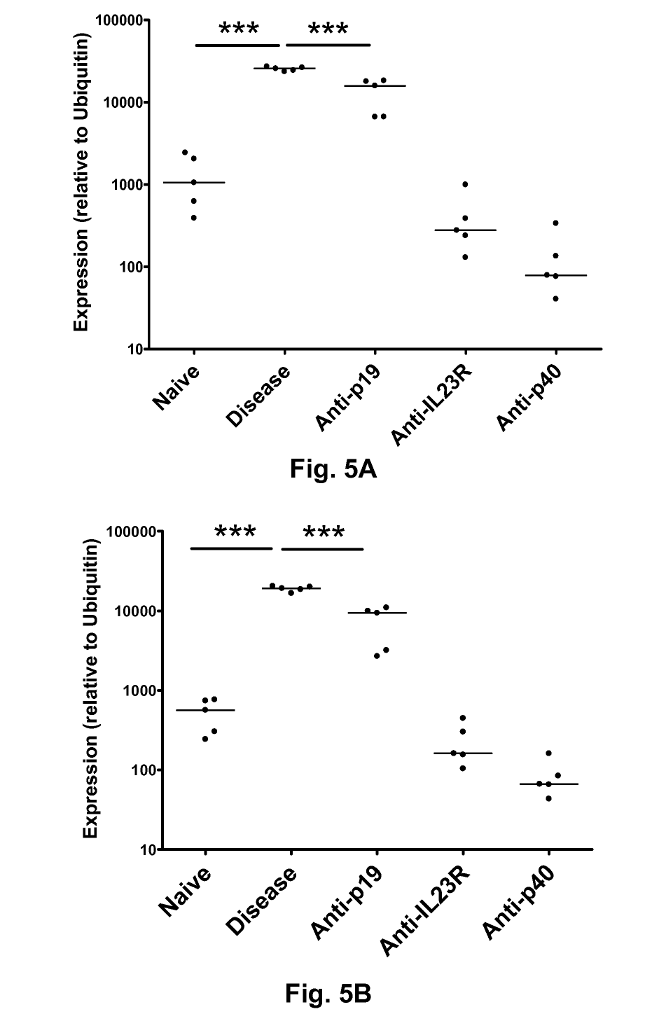

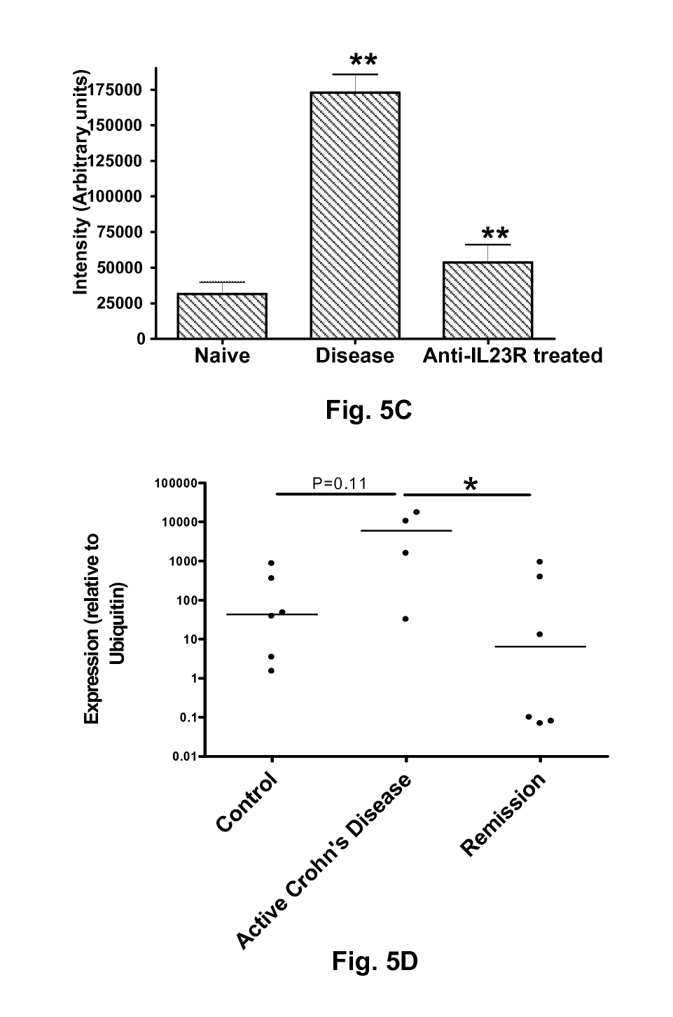

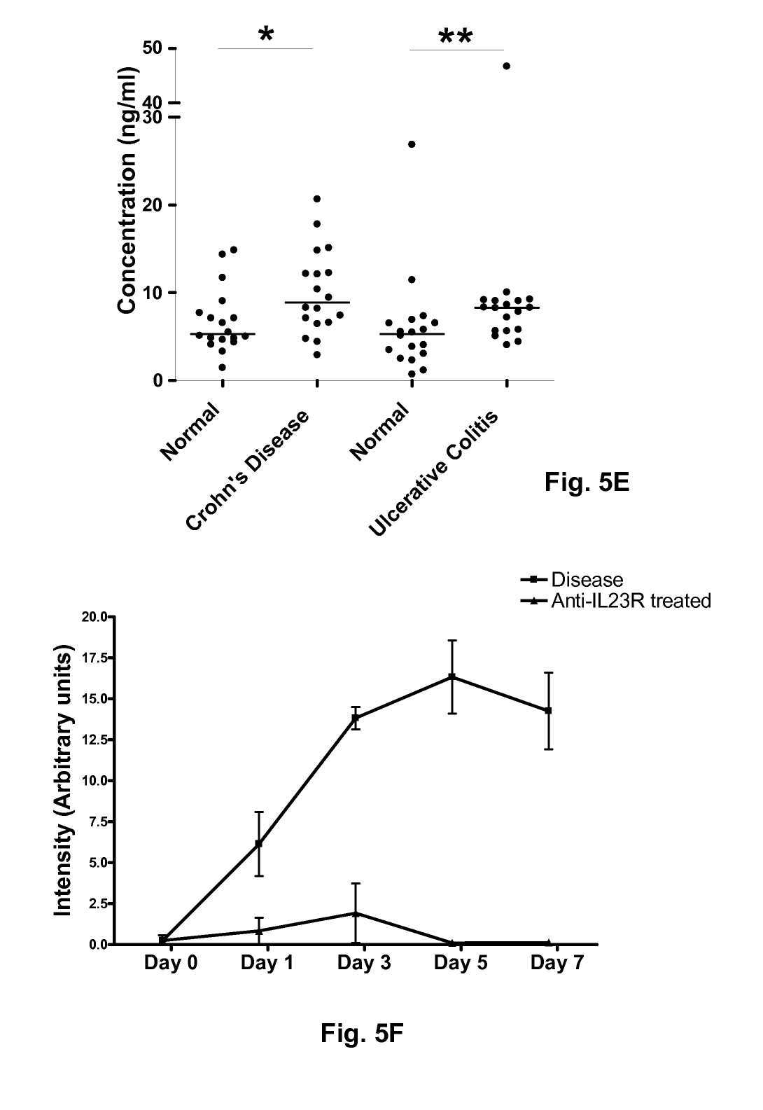

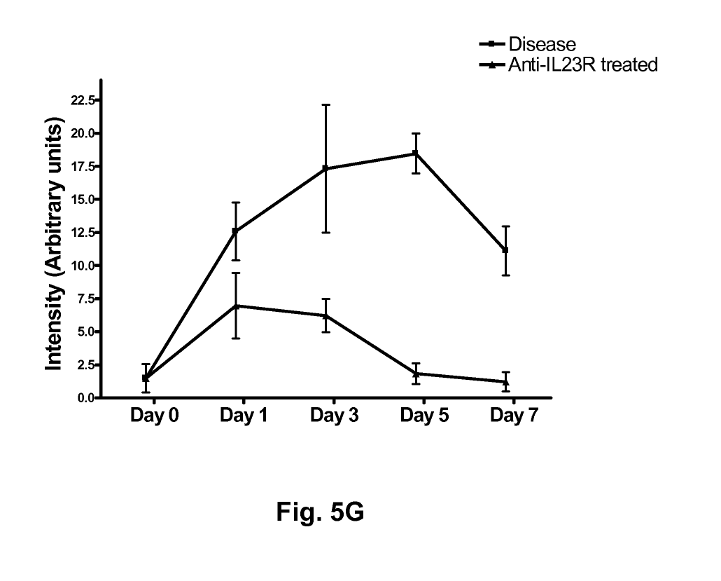

Mouse REG3β and REG3γ gene expression were elevated in IBD mice compared with control mice, and treatment with anti-IL-23R or anti-IL-12/23p40 antibodies (and to a lesser extent anti-IL-23p19 antibody) reverted mouse REG3β and REG3γ to control levels. FIGS. 5A and 5B. A time course study (data not shown) demonstrated that mouse REG3β and REG3γ were both unregulated at Day 1 after anti-CD40 administration, reaching maximal levels at Day 3 onward. Treatment with anti-IL-23R antibody maintained mouse REG3β and REG3γgene expression at approximately pre-disease levels at all times. Reversion of mouse REG3β and REG3γ gene expression was also found to be generally dose responsive, with higher doses of anti-IL-23p19 antibody or anti-IL-23R antibody showing more complete reversion than lower doses (data not shown).

The level of REG3γ protein was also elevated in IBD mice, but reverted to near control levels by treatment with anti-IL-23R antibodies. FIG. 5C.

Human PAP/REG3α was further validated by gene expression analysis of human gut biopsies from active Crohn's disease patients, Crohn's disease patients in remission, and control subjects. FIG. 50. PAP/REG3α expression was elevated in active Crohn's disease, and reverted to control levels in remission. PAP/REG3α was also validated by ELISA of human serum samples from Crohn's disease and ulcerative colitis patients and control subjects. FIG. 5E. Human PAP/REG3α was detected using the PancrePAP Immunoenymatic Kit for Assaying Human PAP from Dynabio S.A. (Marseille, France). The PAP/REG3α level was slightly, but significantly, elevated in both the Crohn's disease and ulcerative colitis samples.

REG3γ levels were also measured over time courses in mouse colonic epithelial cells and in feces. FIGS. 5F and 5G. In both cases, REG3γ increased after anti-CD40 administration to maximal levels at Day 3 onward. In both cases, treatment with anti-IL-23R antibodies was somewhat effective at reducing REG3γ levels at Days 1 and 3, but fully effective to restore REG3γ to control levels at Days 5 and 7.

The results (FIG. 5D) indicate that there is more than a two log (˜150-fold) increase in PAP/REG3α expression levels in Crohn's patients (gut biopsy) compared with Controls or patients in remission. Accordingly, an assay indicating a 100-fold increase in PAP/REG3α expression over normal levels in gut biopsies would be consistent with the presence of Crohn's disease, whereas a two-fold increase over normal levels would not seem particularly significant (even if statistically reliable). Results (FIG. 5E) also indicate that there is less than a log (˜two-fold) increase in serum PAP/REG3α levels in Crohn's patients compared with Controls. Accordingly, an assay indicating a two-fold increase in serum PAP/REG3α over normal levels would be consistent with the presence of Crohn's disease.

The fact that mouse REG3β and REG3γ levels are reflective of disease state in the mouse IBD model suggests that both human REG3 proteins (PAP/REG3α and REG3γ), as closely related members of the same gene family, may also reflect disease state in humans, and thus be useful as IBD biomarkers. Although many of the embodiments described herein recite measurement of the level (or the level of gene expression) of human PAP/REG3α, for example in measurement in feces, human REG3γ may also find use in place of PAP/REG3α in such embodiments. In addition, mouse REG3γ, like calprotectin, is stable for at least several days at room temperature. This suggests that the human orthologs might also be stable, and thus more convenient for use as biomarkers.

In addition, FIGS. 11E and 12E show that REG3β and REG3γ levels are elevated in the mouse T cell transfer colitis model described at Example 18, and that treatment with anti-IL-23p19 antibody dramatically reduces the levels of both as compared to animals treated with an isotype control antibody. This is true for both polypeptides under both the prophylactic (FIG. 11E) and treatment (FIG. 12E) protocols.

### Example 10

### Calprotectin

Calprotectin comprises S100A8 and S100A9 subunits, and is referred to herein as S100A8/A9. The sandwich ELISA to detect calprotectin requires that both subunits be present to constitute a detectable complex, although expression of the genes for the subunits are measured independently of one another by TaqMan® real-time quantitative PCR analysis.

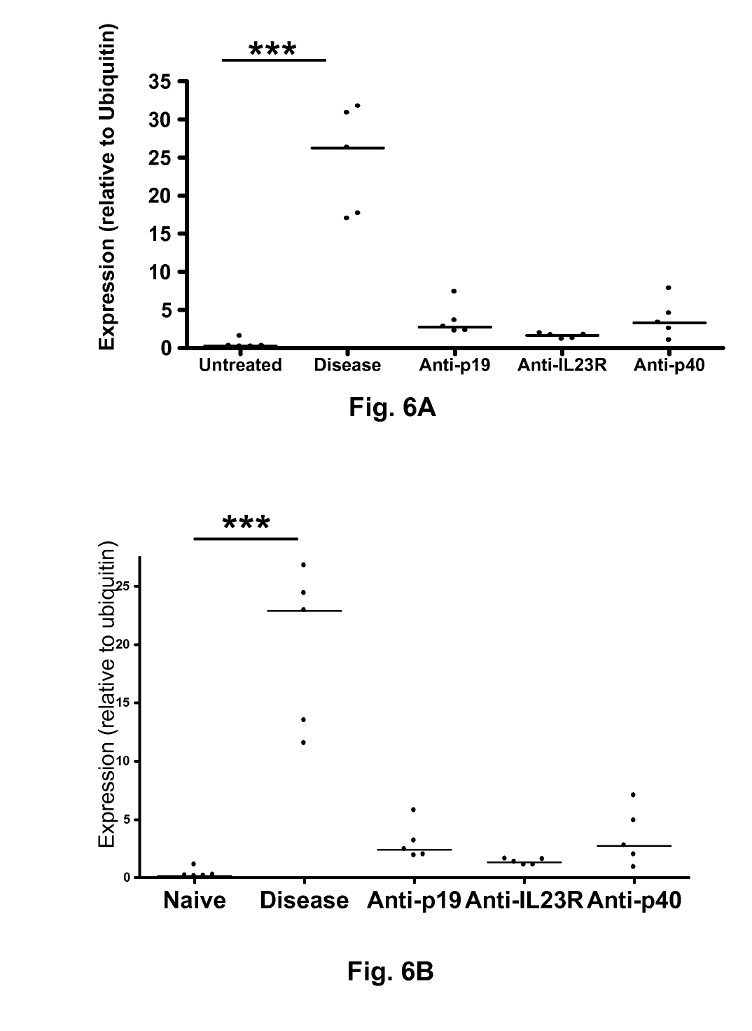

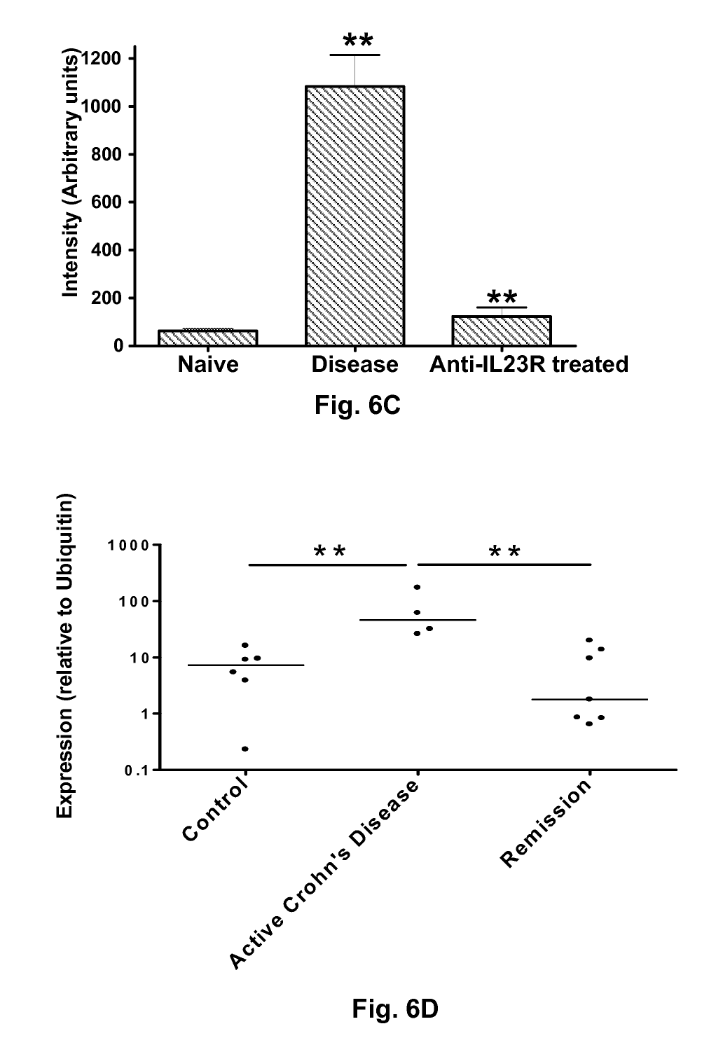

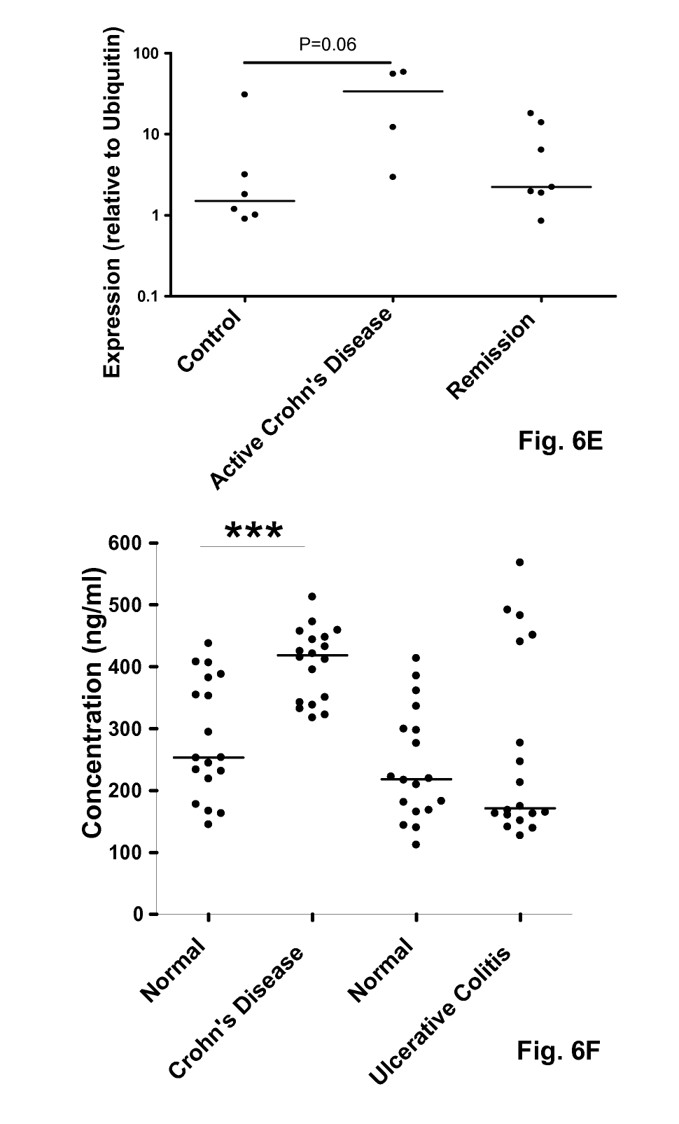

S100A8 and S100A9 gene expression was elevated in IBD mice compared with control mice, and treatment with anti-IL-23R, anti-IL-12/23p40 and anti-IL-23p19 antibodies reverted S100A8 and S100A9 to near control levels. FIGS. 6A and 6B. S100A9 expression was much more dramatically enhanced in the mouse IBD model than S100A8 expression. A time course study (data not shown) demonstrated that S100A8 and S100A9 gene expression in colon were both elevated at Day 1 after anti-CD40 administration, by which each had reached near maximal levels. Treatment with anti-IL-23R antibody had no effect on S100A8 and S100A9 gene expression at Day 1, but reverted S100A8 and S100A9 expression gradually over Days 3 and 5 to nearly pre-disease levels by Day 7. In feces, S100A8 protein levels were not increased at Day 1 but were elevated to maximal levels by Day 3, and treatment with anti-IL-23p19 antibody maintained non-disease levels at all time points (data not shown). Reversion of S100A8 and S100A9 gene expression was also found to be generally dose responsive, with higher doses of anti-IL-23p19 antibody or anti-IL-23R antibody showing more complete reversion than lower doses (data not shown).

The level of S100A8 protein was also elevated in IBD mice, but reverted to near control levels by treatment with anti-IL-23R antibodies. FIG. 6C.

S100A8 and S100A9 were further validated by gene expression analysis of human gut biopsies from active Crohn's disease patients, Crohn's disease patients in remission, and control subjects. FIGS. 6D and 6E. Expression of both S100A8 and S100A9 was elevated in active Crohn's disease, and reverted to control levels in remission. An ELISA showed that S100A8/A9 complex was elevated in serum samples from Crohn's disease patients, and to a lesser extent, ulcerative colitis patients. FIG. 6F. Human calprotectin was detected using the Human Calprotectin ELISA Test Kit (catalog HK325) from HyCult Biotechnology B.V. (Uden, The Netherlands).

The results in FIGS. 6D and 6E indicate that there is about a log (˜10-fold) increase in S100A8 and S100A9 expression levels, respectively, in Crohn's patients (gut biopsy) compared with Controls or patients in remission. Accordingly, an assay indicating a 5-fold increase in S100A8 or S100A9 expression over normal levels in gut biopsies would be consistent with the presence of Crohn's disease, whereas a two-fold increase over normal levels would not seem particularly significant (even if statistically reliable). FIG. 6F indicates that there is less than a 2-fold increase in S100A8/A9 complex levels in serum from Crohn's disease and ulcerative colitis patients compared with controls.

In addition, normal plasma levels of calprotectin are 500-3000 ng/ml, so higher calprotectin levels would suggest IBD. Fecal calprotectin levels have been found to be over 10-fold higher in patients with active ulcerative colitis as compared to subjects with a normal colonoscopy. Røseth et al. (1997) Digestion 58:176.

Calprotectin may also serve as a useful marker to distinguish Crohn's disease from ulcerative colitis. FIG. 6F demonstrates that all Crohn's patients exhibit levels of calprotectin greater than about 300 ng/ml, whereas only 5 of 18 ulcerative colitis subjects do. See Example 29.

In addition, FIGS. 11D and 12D show that calprotectin levels are elevated in the mouse T cell transfer colitis model described at Example 18, and that treatment with anti-IL-23p19 antibody typically dramatically reduces the levels of both S100A8 and S100A9 as compared to animals treated with an isotype control antibody. This is true for both polypeptides under both the prophylactic (FIG. 11D) and treatment (FIG. 12D) protocols.

### Example 11

### IL-22

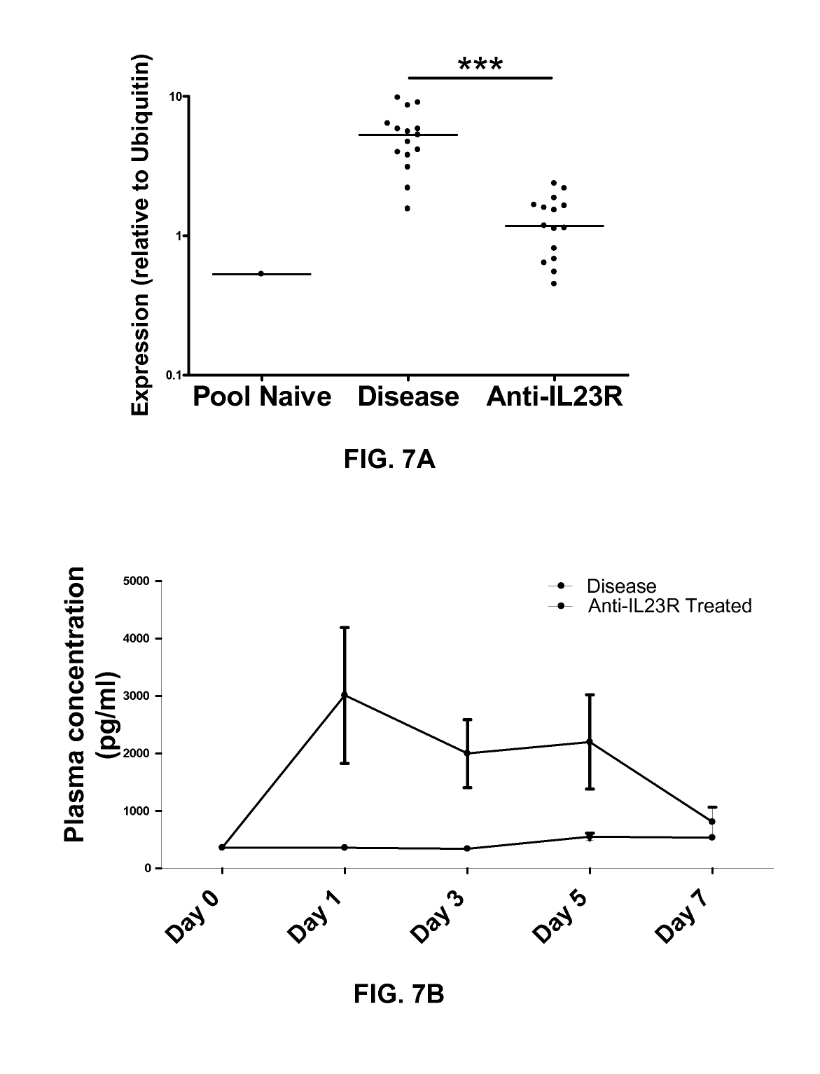

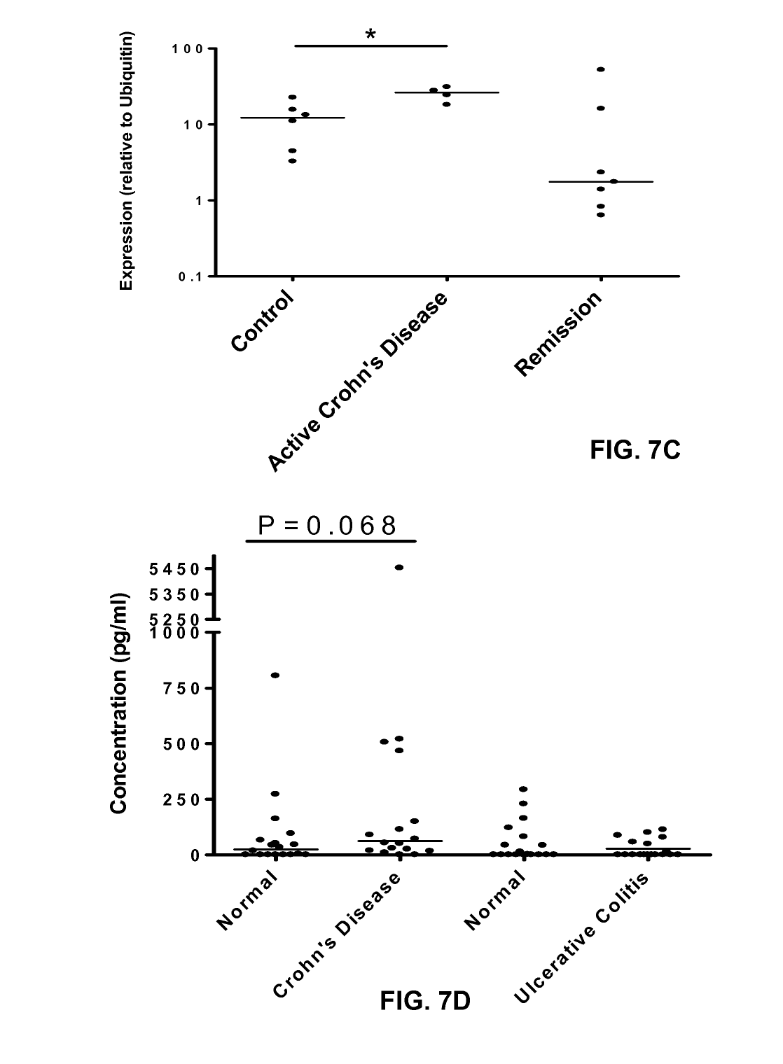

IL-22 gene expression was elevated in IBD mice (whole colon tissue lysate) compared with control mice, and treatment with anti-IL-23R antibody significantly reverted IL-22 toward control levels by Day 7. FIG. 7A. Time course experiments (data not shown) demonstrated that treatment with anti-IL-23R antibody reduced IL-22 gene expression to nearly pre-disease levels at all timepoints. Reversion of IL-22 gene expression by anti-IL-23p19 antibody was also found to be generally dose responsive, with higher doses of antibody showing more complete reversion than lower doses (data not shown). IL-22 was also measured by ELISA in the plasma of IBD mice over several days. FIG. 7B. Anti-IL-23R treatment maintained IL-22 at near-control values at all times.

IL-22 was further validated by gene expression analysis of human gut biopsies from active Crohn's disease patients, Crohn's disease patients in remission, and control subjects. FIG. 7C. IL-22 expression was somewhat elevated in active Crohn's disease, and reverted to control levels (or lower) in remission.

The results (FIG. 7C) indicate that there is less than a half log (˜2-fold) increase in IL-22 expression levels in Crohn's patients (gut biopsy) compared with Controls, although there is an apparent (unexplained) greater difference between active Crohn's patients and those in remission. An assay indicating a two-fold increase in IL-22 expression over normal levels in gut biopsies would be consistent with the presence of Crohn's disease, whereas a 20% increase over normal levels would not seem particularly significant (even if statistically reliable).

The results presented in FIG. 7D demonstrate somewhat higher levels of IL-22 in the serum of Crohn's and ulcerative colitis patients than in the serum of control subjects, as reported at Schmechel et al. (2008) Inflamm. Bowel Dis. 14:204, although these results were not statistically significant. Human IL-22 was detected using the human IL-22 DuoSet® ELISA Development System (catalog DY782) from R&D Systems® (Minneapolis, Minn., USA).

In addition, FIGS. 11A and 12A show that IL-22 levels are elevated in the mouse T cell transfer colitis model described at Example 18, and that treatment with anti-IL-23p19 antibody reduces the level as compared to animals treated with an isotype control antibody under both the prophylactic (FIG. 11A) and treatment (FIG. 12A) protocols.

### Example 12

### Haptoglobin

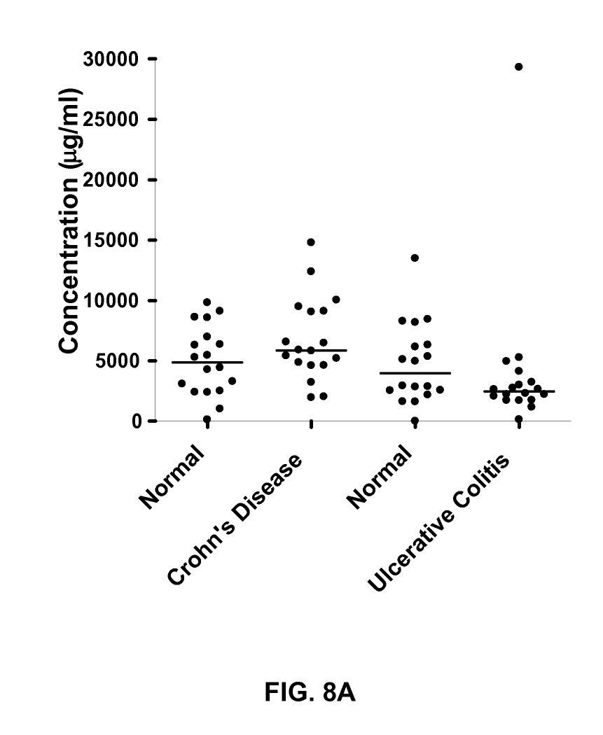

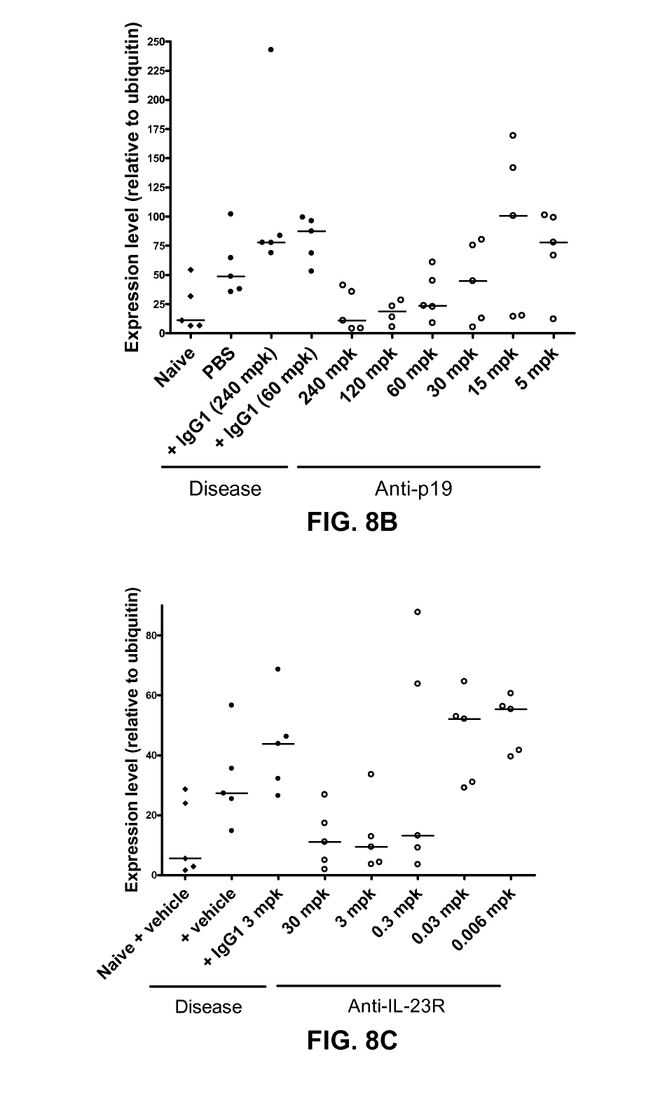

Haptoglobin was found to be insignificantly elevated in human Crohn's disease serum samples, and reduced in ulcerative colitis serum samples, as compared to control samples. FIG. 8A. Human haptoglobin was detected using the Human Haptoglobin ELISA Quantitation Kit (catalog 40-288-20080F) from GenWay Biotech, Inc. (San Diego, Calif., USA). Haptoglobin gene expression was elevated in IBD mice, and reversion of haptoglobin gene expression was generally dose responsive, with higher doses of anti-IL-23p19 antibody or anti-IL-23R antibody showing more complete reversion than lower doses. FIGS. 8B and 8C. In FIGS. 8B and 8C, “naïve” refers to non-disease animals (no anti-CD40 administered) but all other data relate to IBD mice treated with PBS, vehicle or various concentrations of therapeutic or control antibody.

### Example 13

### Validation of Crohn's Disease Biomarker Panel for Serum Comprising PAP/REG3α, LCN2 and CCL20

A serum biomarker panel of the present invention comprising PAP/REG3α, LCN2 and CCL20 was validated against human Crohn's disease samples as follows. Serum samples obtained from 18 Crohn's patients and 18 non-IBD subjects were analyzed by ELISA for the level of PAP/REG3α, LCN2 and CCL20 (essentially as described in previous Examples). Levels were calculated by comparison with standard curve included in each assay. Multivariate discriminant analysis (JMP® Statistical Discovery Software, SAS Institute Inc., Cary, N.C., USA) was used when determine the combination of biomarkers best able to discriminate between Crohn's and non-Crohn's samples.

The combination of PAP/REG3α, LCN2 and CCL20 was found to be particularly predictive of Crohn's disease. LCN2 alone was predictive of Crohn's disease with only 4 samples (11%) miscategorized. When LCN2 and PAP/REG3α results were combined, only 2 samples (5%) were mischaracterized. When LCN2, PAP/REG3α and CCL20 results were combined, only 1 sample (3%) was mischaracterized. Taken as a whole, the biomarker panel of LCN2, PAP/REG3α and CCL20 represents a powerful predictor of Crohn's disease, with 97% reliability.

### Example 14

### Use of Biomarker Panel for Serum Comprising PAP/REG3α, LCN2 and CCL20

Clinical samples are assessed for progression of IBD in a subject during a course of treatment as follows. A subject is identified as having Crohn's disease, referred to herein as the patient Administration of an anti-IL-23p19 humanized monoclonal antibody is selected as the treatment regimen. A pre-treatment blood sample is obtained from the patient during a period of active disease for determination of the levels of PAP/REG3α, LCN2 and CCL20. Samples are then obtained on the first day of administration of the drug (anti-IL-23p19 humanized monoclonal antibody) and weekly thereafter for a period of 12 weeks. The patient is dosed with 2 mg/kg of drug i.v. biweekly for the 12 week period.

Serum is prepared from the blood samples, and biomarker levels are measured by ELISA essentially as described in the preceding Examples. Assays may be performed at any point after the sample is obtained, and need not be performed prior to obtaining a subsequent sample. Briefly, serum is added in duplicate to wells of microtiter plates for PAP/REG3α, LCN2 and CCL20 ELISAs, respectively. Serial dilutions of each protein are included (in duplicate) on the respective ELISA plates for use in constructing a standard curve. After incubation for 30 minutes at room temperature, wells are washed twice with PBS and 100 μl of a detection solution comprising a mouse anti-human (PAP/REG3α, LCN2 or CCL20) IgG detection antibody is added. After a further 30 minute incubation at room temperature, plates are washed twice and 100 μl of a solution comprising a secondary antibody (rabbit anti-mouse IgG-HRP) is added. After a further 30 minute incubation at room temperature, plates are again washed twice and 100 μl of a chromogenic substrate (o-nitrophenyl 8-D-galactoside, ONPG, 5 mM final concentration) is added. Plates are read at 420 nm after 10 minutes of incubation. The resulting O.D. values from the control samples are averaged and used to construct a standard curve, against which the O.D. values for the serum samples are compared to deduce the concentration of the biomarkers in the original blood samples.

If the patient does not exhibit a change in disease state as assessed by changes in the levels of the biomarkers PAP/REG3α, LCN2 or CCL20 at any timepoint (i.e. if the biomarkers still suggest active IBD) then the therapy is considered to have failed. Therapy may be modified, e.g. to increase dosing, or it may be discontinued altogether (e.g. in favor of an alternative treatment option), at the discretion of a medical practitioner and the patient. If, on the other hand, the disease state at the last timepoint is essentially the same as non-disease subject the therapy is considered to be a success, and treatment may be discontinued (e.g. if it is believed that continued therapy is not necessary to maintain disease status), continued unchanged, or modified (e.g. the dose may be somewhat lowered), again at the discretion of a medical practitioner and the patient.

If the disease state at the end of the course of treatment is intermediate between active IBD and non-disease states, treatment is considered to be at least partially successful. Treatment may be continued unchanged (e.g. to allow for potential further improvement over time and/or if symptoms have improved to acceptable levels) or modified (e.g. increasing the dose) or discontinued (e.g. if dosing cannot be increased and symptom relief is insufficient), again at the discretion of a medical practitioner and the patient.

### Example 15

### Use of Biomarker Panel for Serum Comprising IL-22, PAP/REG3α and Calprotectin

A panel of biomarkers of the present invention comprising IL-22, PAP/REG3α and calprotectin is used to monitor therapy in IBD patients essentially as described in Example 14, but with IL-22 and calprotectin rather than LCN2 and CCL20.

Monitoring is effected essentially as follows. A subject believed to suffer from IBD is selected for treatment with an anti-IL-23p19 antibody. A pre-treatment blood sample is obtained from the subject for determination of pre-treatment (untreated) levels of the Biomarkers. The anti-IL-23p19 antibody is then administered intravenously, bi-weekly over a 12 week course of therapy, at 2 mg/kg. Post-treatment blood samples are taken weekly over the course of treatment for determination of Biomarker levels.

Serum samples are prepared from the collected blood samples for use in an ELISA. Serum is added in duplicate to wells of microtiter plates for IL-22, PAP/REG3α and calprotectin ELISAs, respectively. Serial dilutions of each protein are included (in duplicate) on the respective ELISA plates for use in constructing a standard curve. After incubation for 30 minutes at room temperature, wells are washed twice with PBS and 100 μl of a detection solution comprising a mouse anti-human (IL-22, PAP/REG3α or calprotectin) IgG detection antibody is added. After a further 30 minute incubation at room temperature, plates are washed twice and 100 μl of a solution comprising a secondary antibody (rabbit anti-mouse IgG-HRP) is added. After a further 30 minute incubation at room temperature, plates are again washed twice and 100 μl of a chromogenic substrate (o-nitrophenyl 8-D-galactoside, ONPG, 5 mM final concentration) is added. Plates are read at 420 nm after 10 minutes of incubation. The resulting O.D. values from the control samples are averaged and used to construct a standard curve, against which the O.D. values for the serum samples are compared to deduce the concentration of the biomarkers in the original blood samples.

The levels of the biomarkers in post-treatment serum samples are then compared to pre-treatment levels to determine whether treatment caused a reduction in biomarkers toward non-disease levels. Non-disease levels are determined by performing essentially the same biomarker assay method described in this example on samples obtained from non-IBD subjects (i.e. non-disease controls). A ratio of the post-treatment level to a non-disease reference level is calculated for each of IL-22, PAP/REG3α and calprotectin at each timepoint. The data are plotted to determine whether, and the degree to which, the ratio decreases over the course of treatment.

Results of such monitoring are used to assess the patient's response to treatment and modify treatment if necessary, such as increasing dosing or decreasing dosing (including modification of the dosing interval), including discontinuation of treatment, as would be within the skill in the art for a medical practitioner. See Example 14.

### Example 16

### Use of Biomarker Panel for Feces Comprising PAP/REG3α and Calprotectin

A panel of biomarkers of the present invention comprising PAP/REG3α and calprotectin is used to monitor therapy in IBD patients essentially as described in Example 15, except that determinations are based on feces samples rather than blood, and without IL-22.

A 0.1 g sample of feces is vortexed for 30 seconds in 5 ml of PBS supplemented with a COMPLETE™ EDTA free cocktail of protease inhibitors (Roche), and left for 30 minutes on a rotator at room temperature. One ml of the sample is transferred to a 1.5 ml microcentrifuge tube and centrifuged at 10,000 g in an Eppendorf micro centrifuge Model 5415-C (Eppendorf AG, Hamburg, Germany) for 20 minutes. 0.5 ml of the clear supernatants is then prepared to use in an ELISA (e.g. by making appropriate dilutions) or stored up to 3 months at −20° C. for subsequent measurement.

PAP/REG3α and calprotectin ELISAs are then performed essentially as described in previous Examples, and data are analyzed as in Example 14.

### Example 17

### Use of Biomarker Panel for Feces Comprising PAP/REG3α and Calprotectin in a Clinical Trial

Measurement of the levels of PAP/REG3α and Calprotectin is used to assess the therapeutic efficacy of a potential drug (an anti-IL-23 antibody) in a clinical trial as follows.

Biomarkers PAP/REG3α and Calprotectin are measured in fecal samples from subjects essentially as described in Example 16, except that the subjects comprise a control group not receiving a potential drug, a first treatment group comprising subjects receiving the potential drug at a first dose, a second treatment group receiving the potential drug at a higher dose, and a third treatment group receiving the potential drug at the highest dose.

Data are analyzed essentially as in Example 16 to provide values for the concentration of the biomarkers in the samples. The levels of biomarkers are plotted as a function of dose to determine whether the treatment group has lower levels of the biomarkers, indicating that the treatment is efficacious, and optionally whether the therapeutic effect is dose responsive. Reduction of the levels of PAP/REG3α and Calprotectin in treated subjects suggests that a potential drug is efficacious, and a positive dose response (in which higher doses lead to greater reduction in biomarker levels), if observed, bolsters that conclusion. The lack of a dose response, however, is not necessarily evidence of a lack of efficacy of the potential drug.

### Example 18

### Biomarkers in Prophylactic and Therapeutic Protocols Using Mouse T-Cell Transfer IBD Model

To demonstrate the relevance of the biomarkers of the present invention in a the therapeutic context, as opposed to the prophylactic context, gene expression levels were determined in mice with established bowel inflammation in a T-cell transfer IBD model. Such experiments also confirm that the biomarkers of the present invention are useful in a T-cell mediated IBD model, as opposed to the T-cell independent anti-CD40-induced IBD model of Example 3. The anti-CD40-induced IBD model is performed in RAG2 KO mice lacking functional T-cell response, and thus mainly reflects the contribution of the innate immune system to colitis.

The T-cell mediated IBD model used in this example was essentially similar to that described in Elson et al. (2007) Gastroenterology 132:2359, which is hereby incorporated by reference in its entirety. Briefly, colitis was induced by transfer of a cecal bacterial antigen-specific C3H/HeJBir (C3Bir) CD4+ T-cell line (1×10⁶ cells/mouse) intravenously to groups of 3-5 C3H/HeSnJ SCID mice. (The Jackson Laboratory, Bar Harbor, Me., USA). The pathogenic Bir14 CD4+ T-cell established by Elson et al. (Id.) contains IL-17 producing CD4+ T-cells. Anti-CD3 treated T-cells were transferred as a control.

Gene expression levels were determined after adoptive transfer to SCID recipients and establishment of colitis in mice treated with anti-IL-23p19 antibody in either a preventative (prophylactic) or a therapeutic protocol. In the preventative protocol, mice were treated with antibody on the same day as cell transfer and weekly thereafter until sacrifice at week 8. Results are provided at FIGS. 11A-11L. In the therapeutic protocol, mice were treated with antibody weekly from week 4 until sacrifice at week 8. Results are provided at FIGS. 12A-12L. A control group of mice was sacrificed at 4 weeks without antibody treatment to ensure that colitis was present in the Bir14 CD4+ T-cell transfer model. Antibody treatments involved either an anti-IL-23p19 antibody or an isotype control antibody. Antibodies were administered i.p. at 100 μg/mouse/dose. Histopathology was performed on sacrificed mice, and total RNA was obtained from the colon for real-time RT-PCR analysis.

Mice receiving anti-CD3 activated T-cells did not develop colitis, whereas mice that received Bir14 CD4+ T-cell line developed intense colonic inflammation associated with a marked increase of IL-6, GP-39, IL-17, IL-22 and TNF-α gene expression levels, as well as increased expression of S100A8, S100A9, REG3-β, REG3-γ, LCN2 and MIF. Anti-IL-23p19 antibody, administered either prophylactically or therapeutically, reduced expression of many of the biomarkers observed in the anti-CD40 IBD model (Example 3). Both preventative (FIG. 11) and therapeutic (FIG. 12) administration of anti-IL-23p19 antibodies led to downregulation of various of the biomarkers, including TNF-α, IL-17, IL-22, S100A8, S100A9, REG3-β, REG3-γ, LCN2 and MIF. Other biomarkers were also downregulated after anti-IL-23p19 treatment in both preventative and therapeutic protocols, including IL-6, lactoferrin, GPX-2 and GP-39. In contrast, DMBT1 was upregulated in disease but were not reduced by anti-IL-23p19 antibody treatment in either the preventative or therapeutic protocols (data not shown).

The results described in this Example and FIGS. 11 and 12, obtained by measuring gene expression, suggest that the levels of the corresponding proteins would also be increased, making them good biomarkers for disease status and therapeutic response.

### Example 19

### IL-6

IL-6 gene expression levels were determined in the T-cell mouse colitis model described in Example 18. Briefly, T cell transfer mice were either not treated with any antibody, treated with an isotype control antibody, or treated with an anti-IL-23p19 antibody. Antibody was administered in either a preventative mode (i.e. antibody added the same day as T cell transfer) or in a therapeutic mode (i.e. antibody added after onset of colitis, i.e. at 4 weeks pose T cell transfer). Results for preventative and therapeutic treatment experiments are presented at FIGS. 11H and 12H, respectively. In both cases, anti-IL-23p19 antibody reduced. IL-6 expression to near background levels.

For use as a biomarker in humans, IL-6 levels may be determined using a custom-designed ELISA assay, or, for example, using a commercially available ELISA kit, such as the Quantikine® human IL-6 Immunoassay kit (catalog D6050) from R&D Systems® (Minneapolis, Minn., USA).

### Example 20

### IL-17

IL-17 gene expression levels were determined in the T-cell mouse colitis model essentially as described for IL-6 in Example 19. Results for preventative and therapeutic treatment experiments are presented at FIGS. 11B and 12B, respectively. IL-17 expression was somewhat reduced in animals treated with anti-IL-23p19 antibody in preventative mode (FIG. 11B), and slightly reduced in treatment mode (FIG. 12B).

For use as a biomarker in humans, IL-17 levels may be determined using a custom-designed ELISA assay, or, for example, using a commercially available ELISA kit, such as the Quantikine® human IL-17 Immunoassay kit (catalog D1700) from R&D Systems® (Minneapolis, Minn., USA).

### Example 21

### Lactoferrin

Lactoferrin gene expression levels were determined in the T-cell mouse colitis model essentially as described for IL-6 in Example 19. Results for preventative and therapeutic treatment experiments are presented at FIGS. 11I, and 12I, respectively. In both cases, lactoferrin expression was reduced in animals treated with anti-IL-23p19 antibody, although an outlier data point in the treatment data complicates interpretation of the data (FIG. 12I).

For use as a biomarker in humans, lactoferrin levels may be determined using a custom-designed ELISA assay, or, for example, using a commercially available ELISA kit, such as the human lactoferrin ELISA kit (catalog HK329) from HyCult Biotechnology B.V. (Uden, The Netherlands) and the AssayMax human lactoferrin ELBA kit (catalog EL2001-1) from AssayPro (St. Charles, Mo., USA).

### Example 22

### GP-39

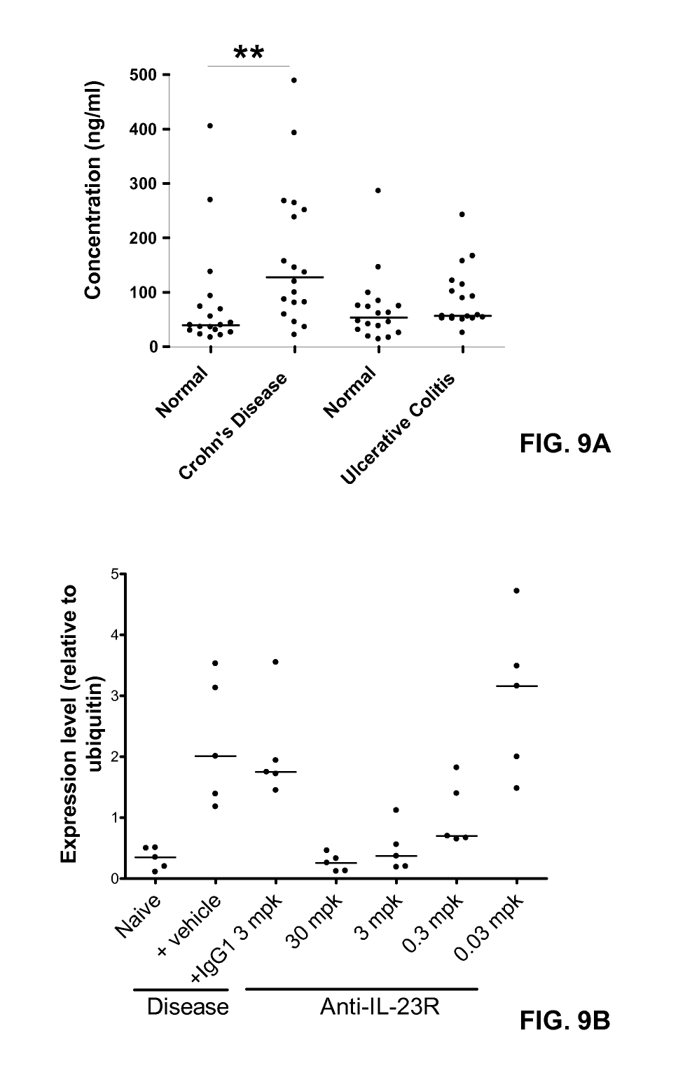

GP-39 was found to be significantly elevated in human Crohn's disease serum samples, and insignificantly elevated in ulcerative colitis serum samples, as compared to control samples. FIG. 9A. GP-39 was detected using the YKL-40 ELISA kit (catalog 8020) from Quidel (San Diego, Calif., USA).

GP-39 gene expression was elevated in IBD mice, and reversion of GP-39 gene expression was generally dose responsive, with higher doses of anti-IL-23R antibody showing more complete reversion than lower doses. FIG. 9B. In FIG. 9B, “naïve” refers to non-disease animals (no anti-CD40 administered) but all other data relate to IBD mice treated with PBS, vehicle or various concentrations of therapeutic or control antibody.

In addition, GP-39 (YKL-40) gene expression levels were determined in the T-cell mouse colitis model essentially as described for IL-6 in Example 19. Results for preventative and therapeutic treatment experiments are presented at FIGS. 11J and 12J, respectively. In both cases, GP-39 expression was reduced in animals treated with an 23p19 antibody.

### Example 23

### GPX-2

GPX-2 gene expression levels were determined in the T-cell mouse colitis model essentially as described for IL-6 in Example 19. Results for preventative and therapeutic treatment experiments are presented at FIGS. 11K, and 12K, respectively. GPX-2 expression was significantly reduced in animals treated with anti-IL-23p19 antibody.

For use as a biomarker in humans, GPX-2 levels may be determined using a custom-designed ELISA assay, or performing a Western blot using a commercially available antibody, such as the monoclonal anti-human GPX2 antibody (catalog MAB5470) from R&D Systems® (Minneapolis, Minn., USA).

### Example 24

### GPX-3

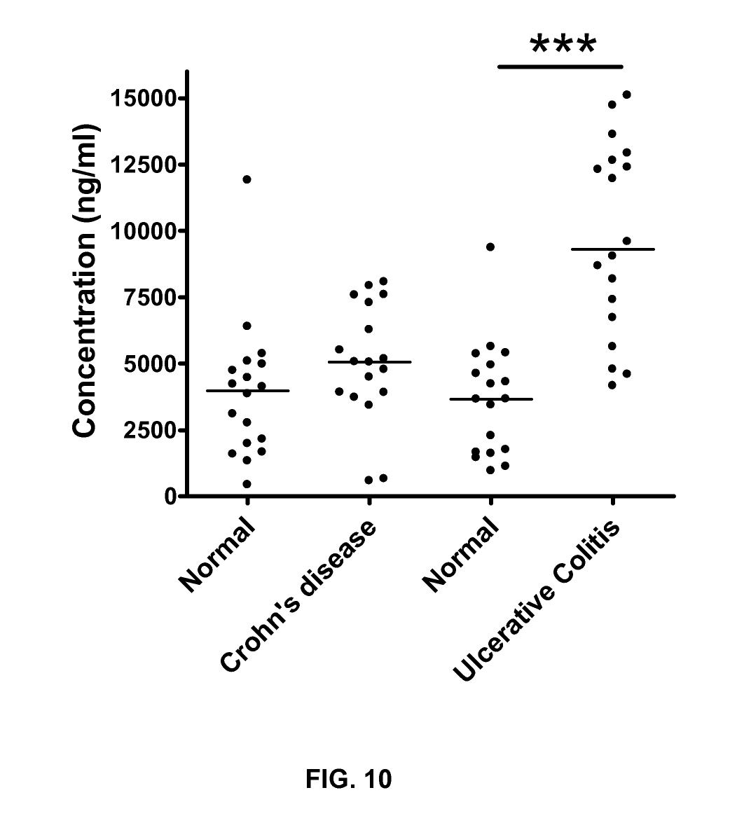

GPX-3 protein levels were determined in human serum samples obtained from Crohn's and UC patients, and from matched normal controls. See Examples 4 and 13. GPX-3 levels are significantly elevated in UC patients, but much less so in Crohn's patients, suggesting that GPX-3 may find use in differential diagnosis of UC versus Crohn's disease. FIG. 10 demonstrates that none of the Crohn's patients exhibit levels of calprotectin greater than about 8000 ng/ml (about twice the levels found in normal subjects), whereas 11 of 18 ulcerative colitis patients do. See Example 29.

GPX-3 was detected using the Glutathione Peroxidase 3 EIA (catalog 44-GPXHU-E01) from Alpco Diagnostics (Salem, New Hanpshire).

### Example 25

### Neutrophil Elastase

Neutrophil elastase gene expression levels were determined in the T-cell mouse colitis model essentially as described for IL-6 in Example 19. Results for preventative and therapeutic treatment experiments are presented at FIGS. 11L, and 12L, respectively. Neutrophil elastase expression was reduced in animals treated with anti-IL-23p19 antibody in a therapeutic protocol (FIG. 12L), but low absolute levels of expression prevent interpretation of the preventative data (FIG. 11L).

For use as a biomarker in humans, neutrophil elastase levels may be determined using a custom-designed ELISA assay, or, for example, using a commercially available ELISA kit, such as the human neutrophil elastase ELISA kit (catalog HK319) from HyCult Biotechnology B.V. (Uden, The Netherlands).

### Example 26

### TNF-α

TNF-α gene expression levels were determined in the T-cell mouse colitis model essentially as described for IL-6 in Example 19. Results for preventative and therapeutic treatment experiments are presented at FIGS. 11C, and 12C, respectively. TNF-α expression was reduced essentially to background levels in animals treated with anti-IL-23p19 antibody, although scatter complicates interpretation of the therapeutic treatment data (FIG. 12C).

For use as a biomarker in humans, TNF-α levels may be determined using a custom-designed ELISA assay, or, for example, using a commercially available ELISA kit, such as the Quantikine® human TNF-α Immunoassay kit (catalog DTA00C) from R&D Systems® (Minneapolis, Minn., USA).

### Example 27

### Validation of Ulcerative Colitis Biomarker Panel for Serum Comprising GPX-3 and MIF

A serum biomarker panel of the present invention comprising GPX-3 and MIF was validated against human ulcerative colitis samples essentially as described at Example 13 (supra), Briefly, serum samples obtained from 18 ulcerative colitis patients and 18 non-IBD subjects were analyzed by ELISA for the level of GPX-3 and MIF (essentially as described in previous Examples). Levels were calculated by comparison with standard curve included in each assay. Multivariate discriminant analysis (JMP® Statistical Discovery Software, SAS Institute Inc., Cary, N.C., USA) was used when determine the combination of biomarkers best able to discriminate between UC and non-UC samples.

The combination of GPX-3 and MIF was found to be particularly predictive of ulcerative colitis. While GPX3 alone was predictive of ulcerative colitis with 7 samples (19%) miscategorized, when results for GPX-3 and MIF were combined only 1 sample (3%) was mischaracterized. Taken as a whole, the biomarker panel of GPX-3 and MIF represents a powerful predictor of ulcerative colitis, with 97% reliability. This panel of UC biomarkers may find use by analogy with the use of panels of Crohn's disease biomarkers discussed at Examples 14-15 (supra).

### Example 28

### Validation of Crohn's Disease Biomarker Panel for Serum Comprising GP-39, CCL20 and/or LCN2

The set of serum biomarkers comprising GP-39, CCL20 and/or LCN2, was determined to be diagnostic for Crohn's disease patients as follows. The same 18 Crohn's and 18 non-Crohn's samples were used as described in Example 13. Serum biomarker protein levels were determined by ELISA. Using decision tree analysis, it was found that samples could be accurately categorized as being from a Crohn's patient (as opposed to a control non-IBD subject) if the level of LCN-2 was greater than about 57 ng/ml, or if the levels of CCL-20 and GP-39 were greater than about 21 pg/ml and 93 ng/ml, respectively.

### Example 29

### Validation of Ulcerative Colitis Biomarker Panel for Serum Comprising LCN2 and MIF

The set of serum biomarkers comprising LCN2 and MIF was determined to be diagnostic for ulcerative colitis patients as follows. The same 18 UC and 18 non-UC samples were used as described in Example 26. Serum biomarker protein levels were determined by ELISA. Using decision tree analysis, it was found that subjects could be accurately categorized as being from an ulcerative colitis patient (as opposed to a control non-IBD subject) if the if the level of MIF was greater than about 4.1 ng/ml and the level of LCN2 was greater than about 6.3 ng/ml.

### Example 30

### Differential Diagnosis of Crohn's Disease and Ulcerative Colitis Using LCN2, Calprotectin (S100A8/A9 complex) and GPX-3

Blood samples are obtained from a statistically significant number of human subjects diagnosed as having Crohn's disease, diagnosed as having ulcerative colitis, and control subject not having any form of inflammatory bowel disorder. Serum is then analyzed for levels of the following biomarker proteins: LCN2, calprotectin (S100A8/A9 complex) and GPX-3. Protein levels are determined by ELISA. The number of subjects necessary to attain statistical significance will depend on the magnitude of the biomarker level differences observed between groups, and the variance in biomarker levels within the groups, and may require adjustment of sample size as is common in studies of this sort. Data are analyzed, for example by decision tree analysis, to determine the levels of the biomarkers that are best able to distinguish Crohn's patients from UC patients.

With these control data in hand, a subject exhibiting symptoms of colitis may be diagnosed as having either Crohn's disease or ulcerative colitis, as follows. A blood sample is obtained from the patient and serum is analyzed for levels of LCN2, calprotectin (S100A8/A9 complex) and GPX-3 by ELISA. Biomarker levels for the subject are then compared with the levels for the serum of known Crohn's and ulcerative colitis patients (as discussed in the preceding paragraph). Samples with biomarker levels similar to the levels in Crohn's disease samples, but dissimilar to levels in ulcerative colitis samples, are indicative of patients with Crohn's disease (and vice versa).

For example, based on the data presented herein, subjects with serum levels of LCN2 above about 50 ngiml are more likely to suffer from Crohn's disease than ulcerative colitis, as are subjects with serum levels of calprotectin (S100A8/A9 complex) above about 300 ng/ml. See FIGS. 4B and 6F. Subjects with serum levels of GPX-3 above about 8000 ng/ml are more likely to suffer from ulcerative colitis than Crohn's disease. See FIG. 10. It is likely that combinations of the above observations would lead to more robust diagnoses. For example, a subject with a combination of two or more of serum levels of >50 ng/ml LCN2, >300 ng/ml calprotectin, and <8000 ng/ml GPX-3 would be particularly likely to suffer from Crohn's disease rather than ulcerative colitis.

Table 5 provides a brief description of the sequences in the sequence listing.

TABLE 5
Sequence Identifiers

| SEQ ID NO: | Description |
|---|---|
| 1 | human IL-23p19 |
| 2 | human IL-23/IL-12p40 |
| 3 | human IL-23R |
| 4 | human IL-12Rβ1 |
| 5 | human CCL20 polynucleotide |
| 6 | human CCL20 polypeptide |
| 7 | human DMBT1 polynucleotide |
| 8 | human DMBT1 polypeptide |
| 9 | human MIF polynucleotide |
| 10 | human MIF polypeptide |
| 11 | human LCN2 polynucleotide |
| 12 | human LCN2 polypeptide |
| 13 | human PAP/REG3α polynucleotide |
| 14 | human PAP/REG3α polypeptide |
| 15 | human REG3γ polynucleotide |
| 16 | human REG3γ polypeptide |
| 17 | human S100A8 polynucleotide |
| 18 | human S100A8 polypeptide |
| 19 | human S100A9 polynucleotide |
| 20 | human S100A9 polypeptide |
| 21 | human IL-22 polynucleotide |
| 22 | human IL-22 polypeptide |
| 23 | human haptoglobin polynucleotide |
| 24 | human haptoglobin polypeptide |
| 25 | human IL-6 polynucleotide |
| 26 | human IL-6 polypeptide |
| 27 | human IL-17 polynucleotide |
| 28 | human IL-17 polypeptide |
| 29 | human lactoferrin polynucleotide |
| 30 | human lactoferrin polypeptide |
| 31 | human GP-39 polynucleotide |
| 32 | human GP-39 polypeptide |
| 33 | human GPX-2 polynucleotide |
| 34 | human GPX-2 polypeptide |
| 35 | human GPX-3 polynucleotide |
| 36 | human GPX-3 polypeptide |
| 37 | human neutrophil elastase polynucleotide |
| 38 | human neutrophil elastase polypeptide |
| 39 | human TNF-α polynucleotide |
| 40 | human TNF-α polypeptide |
| 41 | human C-reactive protein polynucleotide |
| 42 | human C-reactive protein polypeptide |

What is claimed is:

1. A method for detecting the presence or absence of a beneficial response in a patient after administration of an IL-23 antagonist, comprising:
a) obtaining a biological sample from the patient;
b) measuring in said sample the levels of two or more biomarkers, or the levels of expression of two or more biomarkers;
c) comparing the levels to control value of the levels of the biomarkers; and
d) determining whether or not the difference in levels between the sample and the control reflects a beneficial response in the patient,
wherein the two or more biomarkers are selected from the group consisting of calprotectin (a complex of SEQ ID NOs: 18 and 20), PAP/REG3α (SEQ ID NO: 14), MIF (SEQ ID NO: 10), DMBT1 (SEQ ID NO: 8), LCN2 (SEQ ID NO: 12), IL-22 (SEQ ID NO: 22), haptoglobin (SEQ ID NO: 24), CCL20 (SEQ ID NO: 6), CRP (SEQ ID NO: 26), IL-6 (SEQ ID NO: 28), IL-17 (SEQ ID NO: 30) and lactoferrin (SEQ ID NO: 32).

2. The method of claim 1 wherein the levels of biomarker polypeptides are measured.

3. The method of claim 2 wherein the patient suffers from IBD.

4. The method of claim 3 wherein the patient suffers from Crohn's disease.

5. The method of claim 3 wherein the patient suffers from ulcerative colitis.

6. The method of claim 3 wherein the control value is calculated using samples from subjects that do not suffer from IBD.

7. The method of claim 3 wherein the control value is determined using samples from known IBD patients.

8. The method of claim 3 wherein the control value is determined using at least one previous sample taken from the patient.

9. The method of claim 3 wherein the biomarkers are PAP/REG3α, LCN2 and CCL20.

10. The method of claim 3 wherein the biomarkers are calprotectin, PAP/REG3α and IL-22.

11. The method of claim 3 wherein the biomarkers are calprotectin, CRP, IL-6 and IL-22.

12. The method of any of claim 9, 10 or 11 wherein the biological sample is a blood, plasma or serum sample.

13. The method of claim 3 wherein the biomarkers are IL-17, IL-22, lactoferrin, calprotectin and PAP/REG3α.

14. The method of claim 3 wherein the biomarkers are IL-17, lactoferrin and calprotectin.

15. The method of claim 3 wherein the biomarkers are calprotectin and PAP/REG3α.

16. The method of any of claim 13, 14 or 15 wherein the biological sample is a stool sample.

17. The method of claim 3 wherein the IL-23 antagonist is an antibody (or antigen binding fragment thereof) that binds to human IL-23.

18. The method of claim 17 wherein the IL-23 antagonist is an antibody (or antigen binding fragment thereof) that binds to mature human IL-23p19 (residues 20-189 of SEQ ID NO: 1).

19. The method of claim 3 wherein the IL-23 antagonist is an antibody (or antigen binding fragment thereof) that binds to human IL-23 receptor.

20. The method of claim 19 wherein the L-23 antagonist is an antibody (or antigen binding fragment thereof) that binds to mature human IL-23R (residues 24-629 of SEQ ID NO: 3).

21. The method of claim 3 wherein the levels of biomarker polypeptide are determined ELISA.

22. The method of claim 3 wherein the levels of biomarker polypeptide are determined by Western blot.

23. A method of determining whether a potential therapeutic agent is efficacious in the treatment of IBD comprising:
a) obtaining a first biological sample from an IBD patient prior to being treated with the potential therapeutic agent;
b) treating the IBD patient with the potential therapeutic agent;
c) obtaining a second biological sample from the IBD patient after being treated with the potential therapeutic agent;
d) measuring in said first and second sample the levels of two or more biomarkers, or the levels of expression of two or more biomarkers; and
e) comparing the biomarker levels in the second sample to the levels in the first sample, wherein lower biomarker levels in the second sample than in the first sample indicate that the potential therapeutic agent is efficacious,
and further wherein the two or more biomarkers are selected from the group consisting of calprotectin (a complex of SEQ ID NOs: 18 and 20), PAP/REG3α (SEQ ID NO: 14), MIF (SEQ ID NO: 10), DMBT1 (SEQ ID NO: 8), LCN2 (SEQ ID NO: 12), IL-22 (SEQ ID NO: 22), haptoglobin (SEQ ID NO: 24), CCL20 (SEQ ID NO: 6), CRP (SEQ ID NO: 26), IL-6 (SEQ ID NO: 28), IL-17 (SEQ ID NO: 30) and lactoferrin (SEQ ID NO: 32).

24. A method of treating an inflammatory bowel disorder in a subject comprising:
a) determining whether to initiate treatment of the subject, modify the treatment dose, modify the dosing interval, or discontinue treatment, based on the methods of any of the preceding claims; and
b) modifying the treatment regimen based on the determination.

25. An ELISA kit comprising antibodies (or antigen binding fragments thereof) that specifically bind to two or more biomarkers selected from the group consisting of calprotectin complex of SEQ ID NOs: 18 and 20), PAP/REG3α (SEQ ID NO: 14), MIF (SEQ ID NO: 10), DMBT1 (SEQ ID NO: 8), LCN2 (SEQ ID NO: 12), IL-22 (SEQ ID NO: 22), haptoglobin (SEQ ID NO: 24), CCL20 (SEQ ID NO: 6), CRP (SEQ ID NO: 26), IL-6 (SEQ ID NO: 28), IL-17 (SEQ ID NO: 30) and lactoferrin (SEQ ID NO: 32).

26. The ELBA kit of claim 24 further comprising instructions for use of the kit in monitoring IBD.


SEQUENCE LISTING (42 sequences)

SEQ ID NO: 1 - protein, 189 aa, Homo sapiens
  SIGNAL (1)..(19)
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 2 - protein, 328 aa, Homo sapiens
  SIGNAL (1)..(22)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 629 aa, Homo sapiens
  SIGNAL (1)..(23)
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 4 - protein, 662 aa, Homo sapiens
  SIGNAL (1)..(24)
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 5 - DNA, 851 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 6 - protein, 96 aa, Homo sapiens
  SIGNAL (1)..(26)
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 7 - DNA, 7686 bp, Homo sapiens
tttatagcagcagtagaaatataccaccctagaggacacacctccttttagctaggtacctataaatgtccaggattttctattcaattgagaagaacccagcaaaatggggatctccacagtcatccttgaaatgtgtcttttatggggacaagttctatctacaggtgggtggatcccaaggactacagactacgcttcactgattccctcggaggtgcccttggatccaactgtagcagaaggttctccatttccctcggagtcgaccctggagtcaactgtagcagaaggttctccgatttccttggagtcaaccctggagtcaaccgtagcagaaggttctctgattccctcagagtcaaccctggagtcaactgtagcagaaggatctgattctggtttggccctgaggctggtgaatggagatggcaggtgtcagggccgagtggagatcctataccgaggctcctggggcaccgtgtgtgatgacagctgggacaccaatgatgccaacgtggtctgtaggcagctgggttgtggctgggccatgtcagctccaggaaatgcctggtttggccagggctcaggacccattgccctggatgatgtgcgctgctcaggacacgaatcctacctgtggagctgcccccacaatggctggctctcccataactgtggccatggtgaagatgctggtgttatctgctcagctgcccagcctcagtcaacactcaggccagaaagttggcctgtcaggatatcaccacctgtacccacagaaggatctgaatccagtttggccctgaggctggtgaatggaggcgacaggtgtcgaggccgagtggaggtcctataccgaggctcctggggcaccgtgtgtgatgactactgggacaccaatgatgccaatgtggtctgcaggcagctgggctgtggctgggccatgtcagccccaggaaatgcccagtttggccagggctcaggacccattgtcctggatgatgtgcgctgctcaggacatgagtcctacctgtggagctgcccccacaatggctggctcacccacaactgtggccatagtgaagacgctggtgtcatctgctcagctccccagtcccggccgacacccagcccagatacttggccgacctcacatgcatcaacagcaggacctgaatccagtttggccctgaggctggtgaatggaggtgacaggtgtcagggccgagtggaggtcctataccgaggctcctggggcaccgtgtgtgatgatagctgggacaccagtgacgccaatgtggtctgccggcagctgggctgtggctgggccacgtcagccccaggaaatgcccggtttggccagggttcaggacccattgtcctggatgacgtgcgctgctcaggctatgagtcctacctgtggagctgcccccacaatggctggctctcccataactgtcagcacagtgaagacgctggtgtcatctgctcagctgcccactcctggtcgacgcccagtccagacacgttgccgaccatcaccttacctgcatcgacagtaggatctgaatccagtttggccctgaggctggtgaatggaggtgacaggtgtcagggccgagtggaggtcctataccgaggctcctggggcaccgtgtgtgatgacagctgggacaccaatgatgccaatgtggtctgcaggcagctgggctgtggctgggccatgttggccccaggaaatgcccggtttggtcagggctcaggacccattgtcctggatgacgtgcgctgctcagggaatgagtcctacttgtggagctgcccccacaatggctggctctcccataactgtggccatagtgaagacgctggtgtcatctgctcaggacctgaatccagtttggccctgaggctggtgaatggaggtgacaggtgtcagggccgagtggaggtcctataccgaggctcttggggcaccgtgtgtgatgacagctgggacaccaatgatgccaatgtggtctgcaggcagctgggctgtggctgggccacgtcagccccaggaaatgcccggtttggtcagggctcaggacccattgtcctggatgatgtgcgctgctcaggacatgagtcctacctgtggagctgccccaacaatggctggctctcccacaactgtggccatcatgaagatgctggtgtcatctgctcagctgcccagtcccggtcgacgcccaggccagacacgttgtcgaccatcacgttacctccatcgacagtaggatctgaatccagtttgaccctgaggctggtgaatggaagtgacaggtgtcagggccgagtagaggtcctataccgaggctcctggggcaccgtgtgtgatgacagctgggataccaatgatgccaatgtggtctgcaggcagctgggctgtggctgggccacgtcggccccaggaaatgcccggtttggccagggctcaggacccattgttctggatgatgtgcgctgctcaggacacgagtcctacctgtggagctgcccccacaatggctggctctcccacaactgtggccatcatgaagatgctggtgtcatctgctcagtttcccagtcccggccgacacccagtccagatacttggccgacctcacatgcatcaacagcaggacctgaatccagtttggccctgaggctggtgaatggaggtgacaggtgtcagggccgagtggaggtcctataccgaggctcctggggcaccgtgtgtgatgatagctgggacaccagtgacgccaatgtggtctgccggcagctgggctgtggctgggccacgtcagccccaggaaatgcccggtttggccagggttcaggacccattgtcctggatgacgtgcgctgctcaggctatgagtcctacctgtggagctgcccccacaatggctggctctcccataactgtcagcacagtgaagacgctggtgtcatctgctcagctgcccactcctggtcgacgcccagtccagacacattgccgaccatcaccttgcctgcatcgacagtaggatctgaatccagtttggccctgaggctggtgaatggaggtgacaggtgtcagggccgagtggaggtcctataccaaggctcctggggcaccgtgtgcgatgacagctgggacaccaatgatgccaatgtcgtctgcaggcaactgggctgtggctgggccatgtcagccccaggaaatgcccggtttggtcagggctcaggacccattgtcctggatgatgtgcgctgctcaggacacgagtcttacctgtggagctgcccccacaatggctggctctcccacaactgtggccatagtgaagacgctggtgtcatctgctcagcttcccagtcccggccaacacctagtccagacacttggccaacctcacatgcatcaacagcaggatctgaatccagtttggccctgaggctggtgaatggaggtgacaggtgtcagggccgagtggaggtcctataccgaggctcctggggcaccgtgtgtgatgactactgggacaccaatgatgccaatgtggtttgcaggcagctgggctgtggctgggccatgtcagccccaggaaatgcccggtttggccagggttcaggacccattgtcctggatgatgtgcgctgctcaggacatgagtcctatctgtggagctgcccccacaatggctggctctcccacaactgtggccatcatgaagacgctggtgtcatctgctcagcttcccagtcccagccgacacccagcccagacacttggccaacctcacatgcatcaacagcaggatctgaatccagtttggccctgaggctggtgaatggaggtgacaggtgtcagggccgagtggaggtcctataccgaggctcctggggcaccgtgtgtgatgactactgggacaccaatgatgccaatgtggtttgcaggcagctgggctgtggctgggccacgtcagccccaggaaatgcccggtttggccagggttcaggacccattgtcctggatgatgtgcgctgctcaggacatgagtcctatctgtggagctgcccccacaatggctggctctcccacaactgtggccatcatgaagacgctggtgtcatctgctcagcttcccagtcccagccgacacccagcccagacacttggccaacctcacatgcatcaacagcaggatctgaatccagtttggccctgaggctggtgaatggaggtgacaggtgtcagggccgagtggaggtcctataccgaggctcctggggcaccgtgtgtgatgactactgggacaccaatgatgccaatgtggtttgcaggcagctgggctgtggctgggccacgtcagccccaggaaatgcccggtttggccagggttcaggacccattgtcctggatgatgtgcgctgctcaggacatgagtcctatctgtggagctgcccccacaatggctggctctcccacaactgtggccatcatgaagacgctggtgtcatctgctcagcttcccagtcccagccgacacccagcccagacacttggccaacctctcgtgcatcaacagcaggatctgaatccactttggccctgagactggtgaatggaggtgacaggtgtcgaggccgagtggaggtcctataccaaggctcctggggcaccgtgtgtgatgactactgggacaccaatgatgccaacgtggtctgcaggcagctgggctgtggctgggccatgtcagccccaggaaatgcccagtttggccagggctcaggacccattgtcctggatgatgtgcgctgctcaggacacgagtcttacctgtggagctgcccccacaatggctggctctcccacaactgtggccatcatgaagatgctggtgtcatctgctcagctgctcagtcccagtcaacgcccaggccagatacttggctgaccaccaacttaccggcattgacagtaggatctgaatccagtttggctctgaggctggtgaatggaggtgacaggtgtcgaggccgagtggaggtcctgtatcgaggctcctggggaaccgtgtgtgatgacagctgggacaccaatgatgccaatgtggtctgcaggcagctgggctgtggctgggccatgtcggccccaggaaatgcccggtttggccagggctcaggacccattgtcctggatgatgtgcgctgctcagggaatgagtcctacctgtggagctgcccccacaaaggctggctcacccacaactgtggccatcacgaagacgctggtgtcatctgctcagccacccaaataaattctactacgacagattggtggcatccaacaactacaaccactgcaagaccctcttcaaattgtggtggcttcttattctatgccagtgggacattctccagcccatcctaccctgcatactaccccaacaatgctaagtgtgtttgggaaatagaagtgaattctggttatcgcataaacctgggcttcagtaatctgaaattggaggcacaccataactgcagttttgattatgttgaaatctttgatggatcattgaatagcagtctcctgctggggaaaatctgtaatgataccaggcaaatatttacatcttcttacaaccgaatgaccattcactttcgaagtgacatcagtttccaaaacactggctttttggcttggtataactccttcccaagcgatgccaccttgaggttggtcaatttaaattcatcctatggtctatgtgccgggcgtgtagaaatttaccatggtggcacctgggggacagtttgtgatgactcctggaccattcaggaagctgaggtggtctgcagacagctagggtgtggacgtgcagtttcagcccttggaaatgcatattttggctctggctctggccccatcaccctggacgatgtagagtgctcagggacggaatccactctctggcagtgccggaaccgaggctggttctcccacaactgtaatcatcgtgaagatgctggtgtcatctgctcaggaaaccatctatcgacacctgctccttttctcaacatcacccgtccaaacacagattattcctgcggaggcttcctatcccaaccatcaggggacttttccagcccattctatcccgggaactatccaaacaatgccaagtgtgtgtgggacattgaggtgcaaaacaactaccgtgtgactgtgatcttcagagatgtccagcttgaaggtggctgcaactatgattatattgaagttttcgatggcccctaccgcagttcccctctcattgctcgagtttgtgatggggccagaggctccttcacttcttcctccaacttcatgtccattcgcttcatcagtgaccacagcatcacaaggagagggttccgggctgagtactactccagtccctccaatgacagcaccaacctgctctgtctgccaaatcacatgcaagccagtgtgagcaggagctatctccaatccttgggcttttctgccagtgaccttgtcatttccacctggaatggatactacgagtgtcggccccagataacgccgaacctggtgatattcacaattccctactcaggctgcggcaccttcaagcaggcagacaatgacaccatcgactattccaacttcctcacagcagctgtctcaggtggcatcatcaagaggaggacagacctccgtattcacgtcagctgcagaatgcttcagaacacctgggtcgacaccatgtacattgctaatgacaccatccacgttgctaataacaccatccaggtcgaggaagtccagtatggcaattttgacgtgaacatttccttttatacttcctcatctttcttgtatcctgtgaccagccgcccttactacgtggacctgaaccaggacttgtacgttcaggctgaaatcctccattctgatgctgtactgaccttgtttgtggacacctgcgtggcatcaccatactccaatgacttcacgtctttgacttatgatctaatccggagtggatgcgtgagggatgacacctacggaccctactcctcgccatctcttcgcattgcccgcttccggttcagggccttccacttcctgaaccgcttcccctccgtgtacctgcgttgtaaaatggtggtgtgcagagcgtatgacccctcttcccgctgctaccgaggctgtgtgttgaggtcgaagagggatgtgggctcctaccaggaaaaggtggacgtcgtcctgggtcccatccagctgcagacccccccacgccgagaagaggagcctcggtaggtggtcgctctcagaccccactgtccaccggggcgcagacccctgactcggggacttgggatgttcctcttggtgtcatattccaactcagattgagccctacattgtgctgcacctggtcatacggagttgaatcagacctggttcccgcctcccccaaggctcatggtccttggaggacccgttgcagggtgaggtcaagagagttctgacctggatggcccatagacctgacgtcccagaatccatgcttctcatctgcaaaatgaaaatgtcaatacttacttcttagcactgttgagagggttacttacataaaggaattttggtgaaactgc

SEQ ID NO: 8 - protein, 2413 aa, Homo sapiens
  SIGNAL (1)..(25)
MGISTVILEMCLLWGQVLSTGGWIPRTTDYASLIPSEVPLDPTVAEGSPFPSESTLESTVAEGSPISLESTLESTVAEGSLIPSESTLESTVAEGSDSGLALRLVNGDGRCQGRVEILYRGSWGTVCDDSWDTNDANVVCRQLGCGWAMSAPGNAWFGQGSGPIALDDVRCSGHESYLWSCPHNGWLSHNCGHGEDAGVICSAAQPQSTLRPESWPVRISPPVPTEGSESSLALRLVNGGDRCRGRVEVLYRGSWGTVCDDYWDTNDANVVCRQLGCGWAMSAPGNAQFGQGSGPIVLDDVRCSGHESYLWSCPHNGWLTHNCGHSEDAGVICSAPQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYRGSWGTVCDDSWDTNDANVVCRQLGCGWAMLAPGNARFGQGSGPIVLDDVRCSGNESYLWSCPHNGWLSHNCGHSEDAGVICSGPESSLALRLVNGGDRCQGRVEVLYRGSWGTVCDDSWDTNDANVVCRQLGCGWATSAPGNARFGQGSGPIVLDDVRCSGHESYLWSCPNNGWLSHNCGHHEDAGVICSAAQSRSTPRPDTLSTITLPPSTVGSESSLTLRLVNGSDRCQGRVEVLYRGSWGTVCDDSWDTNDANVVCRQLGCGWATSAPGNARFGQGSGPIVLDDVRCSGHESYLWSCPHNGWLSHNCGHHEDAGVICSVSQSRPTPSPDTWPTSHASTAGPESSLALRLVNGGDRCQGRVEVLYRGSWGTVCDDSWDTSDANVVCRQLGCGWATSAPGNARFGQGSGPIVLDDVRCSGYESYLWSCPHNGWLSHNCQHSEDAGVICSAAHSWSTPSPDTLPTITLPASTVGSESSLALRLVNGGDRCQGRVEVLYQGSWGTVCDDSWDTNDANVVCRQLGCGWAMSAPGNARFGQGSGPIVLDDVRCSGHESYLWSCPHNGWLSHNCGHSEDAGVICSASQSRPTPSPDTWPTSHASTAGSESSLALRLVNGGDRCQGRVEVLYRGSWGTVCDDYWDTNDANVVCRQLGCGWAMSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSHASTAGSESSLALRLVNGGDRCQGRVEVLYRGSWGTVCDDYWDTNDANVVCRQLGCGWATSAPGNARFGQGSGPIVLDDVRCSGHESYLWSCPHNGWLSHNCGHHEDAGVICSASQSQPTPSPDTWPTSRASTAGSESTLALRLVNGGDRCRGRVEVLYQGSWGTVCDDYWDTNDANVVCRQLGCGWAMSAPGNAQFGQGSGPIVLDDVRCSGHESYLWSCPHNGWLSHNCGHHEDAGVICSAAQSQSTPRPDTWLTTNLPALTVGSESSLALRLVNGGDRCRGRVEVLYRGSWGTVCDDSWDTNDANVVCRQLGCGWAMSAPGNARFGQGSGPIVLDDVRCSGNESYLWSCPHKGWLTHNCGHHEDAGVICSATQINSTTTDWWHPTTTTTARPSSNCGGFLFYASGTFSSPSYPAYYPNNAKCVWEIEVNSGYRINLGFSNLKLEAHHNCSFDYVEIFDGSLNSSLLLGKICNDTRQIFTSSYNRMTIHFRSDISFQNTGFLAWYNSFPSDATLRLVNLNSSYGLCAGRVEIYHGGTWGTVCDDSWTIQEAEVVCRQLGCGRAVSALGNAYFGSGSGPITLDDVECSGTESTLWQCRNRGWFSHNCNHREDAGVICSGNHLSTPAPFLNITRPNTDYSCGGFLSQPSGDFSSPFYPGNYPNNAKCVWDIEVQNNYRVTVIFRDVQLEGGCNYDYIEVFDGPYRSSPLIARVCDGARGSFTSSSNFMSIRFISDHSITRRGFRAEYYSSPSNDSTNLLCLPNHMQASVSRSYLQSLGFSASDLVISTWNGYYECRPQITPNLVIFTIPYSGCGTFKQADNDTIDYSNFLTAAVSGGIIKRRTDLRIHVSCRMLQNTWVDTMYIANDTIHVANNTIQVEEVQYGNFDVNISFYTSSSFLYPVTSRPYYVDLNQDLYVQAEILHSDAVLTLFVDTCVASPYSNDFTSLTYDLIRSGCVRDDTYGPYSSPSLRIARFRFRAFHFLNRFPSVYLRCKMVVCRAYDPSSRCYRGCVLRSKRDVGSYQEKVDVVLGPIQLQTPPRREEEPR

SEQ ID NO: 9 - DNA, 560 bp, Homo sapiens
accacagtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagac

SEQ ID NO: 10 - protein, 115 aa, Homo sapiens
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 11 - DNA, 840 bp, Homo sapiens
actcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaggccaaaaaaaaaaaaaaaaaa

SEQ ID NO: 12 - protein, 198 aa, Homo sapiens
  SIGNAL (1)..(20)
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 13 - DNA, 1002 bp, Homo sapiens
gggagggtcccttcctcagggagcacaggaactctgagactcagcaagggtgtcctgggagggctcggggatgggagagtacacagattcacaactcattcagaactgtagaagatgatggatgtgaccaagatcactttagtcctaggggactagagaaggaaaatgacatgaggcagtggggtatctgtgtgttctcccactgaccacgctttctttagtgactcctgattgcctcctcaagtcgcagacactatgctgcctcccatggccctgcccagtgtatcttggatgctgctttcctgcctcatgctgctgtctcaggttcaaggtgaagaaccccagagggaactgccctctgcacggatccgctgtcccaaaggctccaaggcctatggctcccactgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtcatacggcctaaggctttaaagagcaataaaatttttagtctgc

SEQ ID NO: 14 - protein, 175 aa, Homo sapiens
  SIGNAL (1)..(26)
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 15 - DNA, 947 bp, Homo sapiens
ccatccctgagatctttttataaaaaacccagtctttgctgaccagacaaagcataccagatctcaccagagagtcctaggggactacagaaggaaaaagacaagaggcagtaggatatctgtgtgtcctcccgctgaccacacttcctttagtgacccgattgcctcctcaagtcgcagacactatgctgcctcccatggccctgcccagtgtgtcctggatgctgctttcctgcctcattctcctgtgtcaggttcaaggtgaagaaacccagaaggaactgccctctccacggatcagctgtcccaaaggctccaaggcctatggctccccctgctatgccttgtttttgtcaccaaaatcctggatggatgcagatctggcttgccagaagcggccctctggaaaactggtgtctgtgctcagtggggctgagggatccttcgtgtcctccctggtgaggagcattagtaacagctattcatacatctggattgggctccatgaccccacacagggctctgagcctgatggagatggatgggagtggagtagcactgatgtgatgaattactttgcatgggagaaaaatccctccaccatcttaaaccctggccactgtgggagcctgtcaagaagcacaggatttctgaagtggaaagattataactgtgatgcaaagttaccctatgtctgcaagttcaaggactagggcaggtgggaagtcagcagcctgagcttggcgtgcagctcatcatggacatgagaccagtgtgaagactcaccctggaagagaatattctccccaaactgccctacctgactaccttgtcatgatcctccttctttttcctttttcttcaccttcatttcaggcttttctctgtcttccatgtcttgagatctcagagaataataataaaaatgttactttatacgtaaaaaa

SEQ ID NO: 16 - protein, 175 aa, Homo sapiens
  SIGNAL (1)..(26)
MLPPMALPSVSWMLLSCLILLCQVQGEETQKELPSPRISCPKGSKAYGSPCYALFLSPKSWMDADLACQKRPSGKLVSVLSGAEGSFVSSLVRSISNSYSYIWIGLHDPTQGSEPDGDGWEWSSTDVMNYFAWEKNPSTILNPGHCGSLSRSTGFLKWKDYNCDAKLPYVCKFKD

SEQ ID NO: 17 - DNA, 428 bp, Homo sapiens
atgtctcttgtcagctgtctttcagaagacctggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcaaaaaaaaaaaaaaaaaaaaa

SEQ ID NO: 18 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 19 - DNA, 586 bp, Homo sapiens
aaacactctgtgtggctcctcggctttgacagagtgcaagacgatgacttgcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccacggccacagtcatggtggccacggccacagccactaatcaggaggccaggccaccctgcctctacccaaccagggccccggggcctgttatgtcaaactgtcttggctgtggggctaggggctggggccaaataaagtctcttcctccaagtcaaaaaaaaaa

SEQ ID NO: 20 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 21 - DNA, 1147 bp, Homo sapiens
cgaccaggttctccttccccagtcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgattttttttaaccaaaaggaagatgggaagccaaactccatcatgatgggtggattccaaatgaacccctgcgttagttacaaaggaaaccaatgccacttttgtttataagaccagaaggtagactttctaagcatagatatttattgataacatttcattgtaactggtgttctatacacagaaaacaatttattttttaaataattgtctttttccataaaaaagattactttccattcctttaggggaaaaaacccctaaatagcttcatgtttccataatcagtactttatatttataaatgtatttattattattataagactgcattttatttatatcattttattaatatggatttatttatagaaacatcattcgatattgctacttgagtgtaaggctaatattgatatttatgacaataattatagagctataacatgtttatttgacctcaataaacacttggatatcc

SEQ ID NO: 22 - protein, 179 aa, Homo sapiens
  SIGNAL (1)..(33)
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 23 - DNA, 1284 bp, Homo sapiens
agatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccataaaaaaaaaaaaaaaaaaaaaaaaaaaaaa

SEQ ID NO: 24 - protein, 347 aa, Homo sapiens
  SIGNAL (1)..(18)
MSALGAVIALLLWGQLFAVDSGNDVTDIADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 25 - DNA, 1201 bp, Homo sapiens
aatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaaaaaaaaaaa

SEQ ID NO: 26 - protein, 212 aa, Homo sapiens
  SIGNAL (1)..(29)
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 27 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 28 - protein, 155 aa, Homo sapiens
  SIGNAL (1)..(23)
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 29 - DNA, 2390 bp, Homo sapiens
agagccttcgtttgccaagtcgcctccagaccgcagacatgaaacttgtcttcctcgtcctgctgttcctcggggccctcggactgtgtctggctggccgtaggaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggcctcccctgctgaaggtggggattgcccatccatctgcttacaattccctgctgtcgtcttagcaagaagtaaaatgagaaattttgttgatattctctccttaaaaaaaaaaaaaaaaaaaaaaaaaaaaaa

SEQ ID NO: 30 - protein, 710 aa, Homo sapiens
  SIGNAL (1)..(19)
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 31 - DNA, 1867 bp, Homo sapiens
cacatagctcagttcccataaaagggctggtttgccgcgtcggggagtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtgaaaaaaaaaaaaaaaaaaaaaaaaaaaaaa

SEQ ID NO: 32 - protein, 383 aa, Homo sapiens
  SIGNAL (1)..(21)
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 33 - DNA, 1024 bp, Homo sapiens
  misc_feature (207)..(207): n = A; TGA codes for selenocysteine
cttcctggctcctccttcctccccacccctctaataggctcataagtgggctcaggcctctctgcggggctcactctgcgcttcaccatggctttcattgccaagtccttctatgacctcagtgccatcagcctggatggggagaaggtagatttcaatacgttccggggcagggccgtgctgattgagaatgtggcttcgctctgnggcacaaccacccgggacttcacccagctcaacgagctgcaatgccgctttcccaggcgcctggtggtccttggcttcccttgcaaccaatttggacatcaggagaactgtcagaatgaggagatcctgaacagtctcaagtatgtccgtcctgggggtggataccagcccaccttcacccttgtccaaaaatgtgaggtgaatgggcagaacgagcatcctgtcttcgcctacctgaaggacaagctcccctacccttatgatgacccattttccctcatgaccgatcccaagctcatcatttggagccctgtgcgccgctcagatgtggcctggaactttgagaagttcctcatagggccggagggagagcccttccgacgctacagccgcaccttcccaaccatcaacattgagcctgacatcaagcgcctccttaaagttgccatatagatgtgaactgctcaacacacagatctcctactccatccagtcctgaggagccttaggatgcagcatgccttcaggagacactgctggacctcagcattcccttgatatcagtccccttcactgcagagccttgcctttcccctctgcctgtttccttttcctctcccaaccctctggttggtgattcaacttgggctccaagacttgggtaagctctgggccttcacagaatgatggcaccttcctaaaccctcatgggtggtgtctgagaggcgtgaagggcctggagccactctgctagaagagaccaataaagggcaggtgtggaaacggcaaaaaaaaaaaaaaaaaaaaaaaaaaaaaa

SEQ ID NO: 34 - protein, 190 aa, Homo sapiens
  MISC_FEATURE (40)..(40): Xaa = selenocytseine
MAFIAKSFYDLSAISLDGEKVDFNTFRGRAVLIENVASLXGTTTRDFTQLNELQCRFPRRLVVLGFPCNQFGHQENCQNEEILNSLKYVRPGGGYQPTFTLVQKCEVNGQNEHPVFAYLKDKLPYPYDDPFSLMTDPKLIIWSPVRRSDVAWNFEKFLIGPEGEPFRRYSRTFPTINIEPDIKRLLKVAI

SEQ ID NO: 35 - DNA, 1779 bp, Homo sapiens
  misc_feature (436)..(436): n = A; TGA codes for selenocysteine
gtcgccccgggacggggaggtggggagctgagggcaagtcgcgcccgcccctgaaatcccagccgcctagcgattggctgcaagggtctcggcttggccgcggattggtcacacccgagggcttgaaaggtggctgggagcgccggacacctcagacggacggtggccagggatcaggcagcggctcaggcgaccctgagtgtgcccccaccccgccatggcccggctgctgcaggcgtcctgcctgctttccctgctcctggccggcttcgtctcgcagagccggggacaagagaagtcgaagatggactgccatggtggcataagtggcaccatttacgagtacggagccctcaccattgatggggaggagtacatccccttcaagcagtatgctggcaaatacgtcctctttgtcaacgtggccagctactgnggcctgacgggccagtacattgaactgaatgcactacaggaagagcttgcaccattcggtctggtcattctgggctttccctgcaaccaatttggaaaacaggaaccaggagagaactcagagatccttcctaccctcaagtatgtccgaccaggtggaggctttgtccctaatttccagctctttgagaaaggggatgtcaatggagagaaagagcagaaattctacactttcctaaagaactcctgtcctcccacctcggagctcctgggtacatctgaccgcctcttctgggaacccatgaaggttcacgacatccgctggaactttgagaagttcctggtggggccagatggtatacccatcatgcgctggcaccaccggaccacggtcagcaacgtcaagatggacatcctgtcctacatgaggcggcaggcagccctgggggtcaagaggaagtaactgaaggccgtctcatcccatgtccaccatgtaggggagggactttgttcaggaagaaatccgtgtctccaaccacactatctacccatcacagacccctttcctatcactcaaggccccagcctggcacaaatggatgcatacagttctgtgtactgccaggcatgtgggtgtgggtgcatgtgggtgtttacacacatgcctacaggtatgcgtgattgtgtgtgtgtgcatgggtgtacagccacgtgtctacctatgtgtctttctgggaatgtgtaccatctgtgtgcctgcagctgtgtagtgctggacagtgacaaccctttctctccagttctccactccaatgataatagttcacttacacctaaacccaaaggaaaaaccagctctaggtccaattgttctgctctaactgatacctcaaccttggggccagcatctcccactgcctccaaatattagtaactatgactgacgtccccagaagtttctgggtctaccacactccccaaccccccactcctacttcctgaagggccctcccaaggctacatccccaccccacagttctccctgagagagatcaacctccctgagatcaaccaaggcagatgtgacagcaagggccacggaccccatggcaggggtggcgtcttcatgagggaggggcccaaagcccttgtgggcggacctcccctgagcctgtctgaggggccagcccttagtgcattcaggctaaggcccctgggcagggatgccacccctgctccttcggaggacgtgccctcacccctcactggtccactggcttgagactcaccccgtctgcccagtaaaagcctttctgcagcagctgaaaaaaaaaaaaaaaaaaa

SEQ ID NO: 36 - protein, 226 aa, Homo sapiens
  MISC_FEATURE (73)..(73): Xaa = selenocysteine
MARLLQASCLLSLLLAGFVSQSRGQEKSKMDCHGGISGTIYEYGALTIDGEEYIPFKQYAGKYVLFVNVASYXGLTGQYIELNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 37 - DNA, 938 bp, Homo sapiens
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgtagaaaaaaaaaaaaaaaa

SEQ ID NO: 38 - protein, 267 aa, Homo sapiens
  SIGNAL (1)..(29)
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 39 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 40 - protein, 233 aa, Homo sapiens
  mat_peptide (77)..(233)
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 41 - DNA, 2024 bp, Homo sapiens
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaaggtacctcccggttttttacaccgcatgggccccacgtctctgtctctggtacctcccgcttttttacactgcatggttcccacgtctctgtctctgggcctttgttcccctatatgcattgcaggcctgctccaccctcctcagcgcctgagaatggaggtaaagtgtctggtctgggagctcgttaactatgctgggaaacggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagggcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgaaaaaaaaaa

SEQ ID NO: 42 - protein, 224 aa, Homo sapiens
  SIGNAL (1)..(18)
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP